(19) United States
(12) Patent Application Publication
(10) Pub. No.: US 2024/0139089 A1
(43) Pub. Date: May 2, 2024

(54) MULTISOME LIPID VESICLES FOR DELIVERY OF COSMETIC AGENTS

(71) Applicant: GLO Pharma, Inc., Carlsbad, CA (US)

(72) Inventors: Robert A. LOVE, Encinitas, CA (US); Marianna FOLDVARI, Kitchener (CA)

(21) Appl. No.: 18/473,078

(22) Filed: Sep. 22, 2023

Related U.S. Application Data
(63) Continuation of application No. PCT/US2022/021585, filed on Mar. 23, 2022.
(60) Provisional application No. 63/285,860, filed on Dec. 3, 2021.
(60) Provisional application No. 63/271,645, filed on Oct. 25, 2021.
(60) Provisional application No. 63/165,603, filed on Mar. 24, 2021.

(51) Int. Cl. A61K 8/64; A61K 8/06; A61K 8/14; A61K 8/60; A61Q 19/08

(52) U.S. Cl. CPC A61K 8/64; A61K 8/062; A61K 8/14; A61K 8/60; A61Q 19/08

(57) ABSTRACT

The present invention relates to compositions comprising and methods for use of formulations for the delivery of cosmetic agents, including anionic polymers such as hyaluronic acid and peptides with cosmetic activity. They are useful in, e.g., cosmetics and pharmaceuticals that prevent or improve the appearance of undesirable skin features including wrinkles or enhancing lip fullness.

### CROSS-REFERENCE

This application is a continuation of International Patent Application No. PCT/US22/21585, filed Mar. 23, 2022, which claims the benefit of U.S. Provisional Application No. 63/285,860, filed Dec. 3, 2021, U.S. Provisional Application No. 63/271,645, filed Oct. 25, 2021, and U.S. Provisional Application No. 63/165,603, filed Mar. 24, 2021, all of which are incorporated herein by reference in its entirety.

### BACKGROUND OF THE INVENTION

Certain cosmetic agents are desirably delivered below the surface of the skin. There exists a need for improved methods of delivery and compositions for such cosmetic agents for various cosmetic and pharmaceutical purposes, including the prevention, reduction, or elimination of wrinkles.

### SEQUENCE LISTING

The instant application contains a Sequence Listing which has been submitted electronically in XML format and is hereby incorporated by reference in its entirety. Said XML copy, created on Sep. 20, 2023, is named 54780-715.301_SL.xml and is 196,608 bytes in size.

### SUMMARY OF THE INVENTION

The present invention relates to compositions for delivery of cosmetic agents. In some embodiments, the compositions are lipid vesicle formulations of the cosmetic agents which allow the agents to be delivered below the surface of the skin upon topical application. In some embodiments, the cosmetic agent is an anionic polymer material, such as hyaluronic acid, which is beneficial for the appearance of the skin, such as the skin of the lips of a subject. In some embodiments, the cosmetic agent is a peptide antagonist of muscle-type nicotinic acetylcholine receptors. In some embodiments, the cosmetic agents are delivered to a preferred or pre-selected layer of the skin or surrounding tissue, such as the epidermis, dermis, subcutaneous tissue, or muscle tissue.

In one aspect, provided herein, is a lipid vesicle composition comprising: (a) lipid vesicles each comprising a lipid bilayer comprising vesicle forming lipids; (b) an oil-in-water emulsion entrapped in the lipid vesicles, and stabilized by one or more surfactants; and (c) a peptide antagonist of muscle-type nicotinic acetylcholine receptors entrapped in the lipid bilayer and/or the oil-in-water emulsion. In some embodiments, the peptide antagonist comprises an amino acid sequence at least about 50%, at least about 60%, at least about 70%, at least about 80%, or at least about 90% sequence homology to the amino acid sequence of any one of SEQ ID NOs: 1-52 or 60-99. In some embodiments, the peptide antagonist comprises an amino acid sequence identical to the amino acid sequence of any one of SEQ ID NOs: 1-52 or 60-99. In some embodiments, the peptide antagonist has an amino acid sequence consisting of an identical sequence to the amino acid sequence of any one of SEQ ID NOs: 1-52 or 60-99. In some embodiments, the peptide antagonist has an amino acid sequence consisting of an identical sequence to the amino acid sequence of SEQ ID NO: 60, 61, 73, 78, 82, 85, 91, or 95. In some embodiments, the peptide antagonist has an amino acid sequence consisting of an identical sequence to the amino acid sequence of SEQ ID NO: 60. In some embodiments, the peptide antagonist has an amino acid sequence consisting of an identical sequence to the amino acid sequence of SEQ ID NO: 61. In some embodiments, the peptide antagonist has an amino acid sequence consisting of an identical sequence to the amino acid sequence of SEQ ID NO: 73. In some embodiments, the peptide antagonist has an amino acid sequence consisting of an identical sequence to the amino acid sequence of SEQ ID NO: 78. In some embodiments, the peptide antagonist has an amino acid sequence consisting of an identical sequence to the amino acid sequence of SEQ ID NO: 82. In some embodiments, the peptide antagonist has an amino acid sequence consisting of an identical sequence to the amino acid sequence of SEQ ID NO: 85. In some embodiments, the peptide antagonist has an amino acid sequence consisting of an identical sequence to the amino acid sequence of SEQ ID NO: 91. In some embodiments, the peptide antagonist has an amino acid sequence consisting of an identical sequence to the amino acid sequence of SEQ ID NO: 95. In some embodiments, the peptide antagonist is present at a concentration of from about 0.1 mg/mL to about 10 mg/mL. In some embodiments, the composition further comprises one or more penetration enhancing agents. In some embodiments, the one or more penetration enhancing agents comprises a non-ionic surfactant or a combination of non-ionic surfactants. In some embodiments, the non-ionic surfactant or combination of non-ionic surfactants is selected from polyethylene glycol ethers of fatty alcohols, sorbitan esters, polysorbates, sorbitan esters and polyethylene glycol fatty acid esters and combinations thereof. In some embodiments, the polyethylene glycol ethers of fatty alcohols comprise a C₈-C₂₂ fatty alcohol and a polyethylene glycol group having from about 2 to about 8 ethylene glycol subunits. In some embodiments, the polyethylene glycol ethers of fatty alcohols comprise diethylene glycol hexadecyl ether, 2-(2-octadecoxyethoxy)ethanol, diethylene glycol monooleyl ether, polyoxyethylene (3) oleyl ether, or polyoxyethylene (5) oleyl ether, or any combination thereof. In some embodiments, the sorbitan esters comprise sorbitan monolaurate, sorbitan monopalmitate, sorbitan monostearate, sorbitan monooleate, sorbitan trioleate, sorbitan sesquioleate, or sorbitan isostearate, or any combinations thereof. In some embodiments, the polyethylene glycol fatty acid ester comprises PEG-8 dilaurate, PEG-4 dilaurate, PEG-4 laurate, PEG-8 dioleate, PEG-8 distearate, PEG-8 distearate, PEG-7 glyceryl cocoate, and PEG-20 almond glycerides, or any combination thereof. In some embodiments, the polysorbate comprises polysorbate 20, polysorbate 21, polysorbate 40, polysorbate 60, polysorbate 80, polysorbate 85, or any combination thereof. In some embodiments, each of the non-ionic surfactants has a hydrophobic-lipophilic balance (HLB) of about 10 or less. In some embodiments, the non-ionic surfactant or combination of non-ionic surfactants is present in an amount of from about 0.5% to about to about 10% (w/w) of the composition. In some embodiments, at least one non-ionic surfactant is present in the oil-in-water emulsion. In some embodiments, at least one non-ionic surfactant is present in the lipid bilayer. In some embodiments, the one or more penetration enhancing agents comprises a combination of a sorbitan ester, a polysorbate, and a polyethylene glycol fatty acid esters. In some embodiments, the one or more penetration enhancing agents comprises a combination of a polyethylene glycol ether of a fatty alcohol, a sorbitan esters, and a polysorbate. In some embodiments, the one or more penetration enhancing agents comprises monolauroyllysine or dipalmitoyllysine, or a combination thereof. In some embodiments, the vesicle forming lipids comprise phospholipids, glycolipids, lecithins, ceramides, lysolecithin, lysophosphatidylethanolamine, phosphatidylserine, phosphatidylinositol, sphingomyelin, cardiolipin, phosphatidic acid, cerebroside, or any combination thereof. In some embodiments, the vesicle forming lipids comprise phospholipids. In some embodiments, the composition comprises vesicle forming lipids in an amount of from about 0.5% to about 25% (w/w) of the composition. In some embodiments, the composition comprises a cationic surfactant. In some embodiments, the cationic surfactant is a mono-cationic surfactant. In some embodiments, the cationic surfactant comprises a fatty amide derived propylene glycol-diammonium phosphate ester. In some embodiments, the cationic surfactant is present in an amount of from about 1% to about 10%. In some embodiments, the oil-in-water emulsion comprises a triglyceride in the oil component. In some embodiments, the triglyceride comprises a medium-chain triglyceride. In some embodiments, the triglyceride is present in an amount of from about 1% to about 35% (w/w) of the composition. In some embodiments, the composition comprises a sterol. In some embodiments, the sterol is present in an amount of from about 1% to about 5% (w/w) of the composition. In some embodiments, the composition comprises propylene glycol. In some embodiments, the propylene glycol is present in an amount of from about 1% to about 25% (w/w) of the composition. In some embodiments, the composition comprises one or more viscosity enhancing agents. In some embodiments, the one or more viscosity enhancing agents are present in an amount of from about 0.5% to about 10% (w/w) of the composition. In some embodiments, the composition further comprises one or more additional agents. In some embodiments, the additional agents comprise one or more of a thickener, a preservative, a moisturizer, an emollient, a humectant, an antimicrobial, or any combination thereof. In some embodiments, the composition is formulated for topical application to the skin of a subject. In some embodiments, the composition is formulated to deliver the peptide antagonist to a specified layer of the skin of a subject. In some embodiments, the composition is formulated as a cream, a lotion, a suspension, or an emulsion.

In one aspect, provided herein, is a method of preparing a lipid vesicle composition provided herein, comprising: a) preparing an oil-in-water emulsion comprising the peptide antagonist of muscle-type nicotinic acetylcholine receptors, by mixing oil components of the oil-in-water emulsion with aqueous components of the oil-in-water emulsion, wherein the oil components and/or the aqueous components of the oil-in-water emulsion comprises the one or more surfactants; b) solubilizing vesicle forming lipids in an acceptable solvent other than water; c) adding the oil-in-water emulsion to the solubilized vesicle forming lipids; and d) mixing the oil-in-water emulsion and the solubilized vesicle forming lipids under mixing conditions effective to form the lipid vesicles comprising a lipid bilayer comprising vesicle forming lipids, and an oil-in-water emulsion entrapped in the lipid vesicles.

In one aspect, provided herein, is a method of producing one or more cosmetic effects by delivering a cosmetic agent below a skin surface of a subject, comprising administering to the skin surface a lipid vesicle composition provided herein. In some embodiments, the cosmetic agent is the polyanionic filler material. In some embodiments, the cosmetic agent is delivered to the dermis of the subject. In some embodiments, the one or more cosmetic effects comprises an enhancement of lip fullness, lip volume, lip smoothness, lip color, or a combination thereof. In some embodiments, the cosmetic agent is the peptide antagonist of muscle-type nicotinic acetylcholine receptors. In some embodiments, the cosmetic agent is delivered to muscle or subcutaneous tissue of the subject. In some embodiments, the one or more cosmetic effect comprises prevention or temporary improvement of the appearance of one or more of skin wrinkles. In some embodiments, the one or more skin wrinkles comprises moderate to severe glabellar lines associated with corrugator and/or procerus muscle activity, moderate to severe lateral canthal lines associated with orbicularis oculi activity (crow's feet lines), or moderate to severe forehead lines associated with frontalis muscle activity.

In one aspect, provided herein, is a method of enhancing a lip characteristic in an individual comprising applying a composition to lips of the individual, wherein the composition comprises a lipid vesicle comprising an oil-in-water emulsion and an anionic polymer material. In some embodiments, the composition comprises lipid vesicles comprising an oil-in-water emulsion and an anionic polymer material. In some embodiments, the lipid vesicle is formulated according to methods described herein. In some embodiments, the lipid vesicle comprises phospholipids, surfactants, polymers, emulsifiers, penetration enhancing agents, triglycerides, sterols, or any other materials described herein. In some embodiments, the anionic polymer material comprises an anionic polysaccharide. In some embodiments, the anionic polymer material is hyaluronic acid. In some embodiments, the lip characteristic comprises lip fullness, lip volume, lip smoothness, lip color, or a combination thereof. In some embodiments, the composition is formulated for topical use. In some embodiments, the composition is delivered below a skin surface of the individual. In some embodiments, the composition is delivered below a skin surface of the lips of the individual.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1C shows physicochemical characterization of multisome formulations F1, F2 and F3. Panel A shows confocal microscopic images of multisome formulations F1, F2 and F3 containing a rhodamine red labelled HA250K and green FITC-HATOK; F1 tracings show co-localization of the two labels in the vesicles. FIG. 1B shows light microscopic images of multisome formulations F1, F2 and F3. FIG. 1C shows particle size distribution of multisome formulations F1, F2 and F3.

FIG. 2 shows confocal microscopic images of human skin treated with cationic multisome formulations. Cationic multisome formulations were prepared with a rhodamine red labelled HA250K and green FITC-HAOK or FITC-HA50K. FI tracing show the levels of rhodamine red labelled HA250K and green FITC-HATOK or FITC-HA50K in the skin layers from the surface of the skin to the upper dermis. The plain of the tracing direction is shown on each micrograph.

FIG. 3 shows confocal microscopic images of human skin treated with multisome formulations. Multisome formulations were prepared with a rhodamine red labelled HA250K and green FITC-HA10K or FITC-HA50K. FI tracing show the levels of rhodamine red labelled HA250K and green FITC-HA10K or FITC-HA50K in the skin layers from the surface of the skin to the upper dermis. The plane of the tracing direction is shown on each micrograph.

FIG. 4 shows confocal microscopic images of human skin treated with multisome formulations. Multisome formulations were prepared with a rhodamine red labelled HA250K and green FITC-HA10K or FITC-HA50K. FI tracing show the levels of rhodamine red labelled HA250K and green FITC-HA10K or FITC-HA50K in the skin layers from the surface of the skin to the upper dermis. The plane of the tracing direction is shown on each micrograph.

FIG. 5 shows light microscopic images of multiphasic vesicle systems prepared with C7 peptide and their respective blank (no peptide) formulations. Bar: 10 μm.

FIG. 6A shows particle size distribution (left three graphs) and zeta potential (right three graphs) of C7 peptide multiphasic vesicle delivery systems.

FIG. 6B shows particle size distribution (left three graphs) and zeta potential (right three graphs) of blank multiphasic vesicle delivery systems.

FIG. 7 shows an exemplary pictorial workflow for the preparation of lipid vesicles provided herein.

FIG. 8 shows an exemplary workflow for the preparation of lipid vesicles comprising hyaluronic acid (HA) as provided herein.

FIG. 9 shows results of the topical application of lipid vesicles comprising HA to lips of subjects.

FIGS. 10A-F show organoleptic properties and microscopy images of lipid vesicle formulations.

FIGS. 11A-B show confocal microscopy images of lipid vesicle formulations.

FIG. 12 shows confocal microscopy images of lead lipid vesicle formulations.

### DETAILED DESCRIPTION OF THE INVENTION

### Definitions

As used herein, the term “comprise” or variations thereof such as “comprises” or “comprising” are to be read to indicate the inclusion of any recited feature but not the exclusion of any other features. Thus, as used herein, the term “comprising” is inclusive and does not exclude additional, unrecited features. In some embodiments of any of the compositions and methods provided herein, “comprising” may be replaced with “consisting essentially of” or “consisting of” The phrase “consisting essentially of” is used herein to require the specified feature(s) as well as those which do not materially affect the character or function of the claimed invention. As used herein, the term “consisting” is used to indicate the presence of the recited feature alone.

As used in the specification and appended claims, unless specified to the contrary, the following terms have the meaning indicated below.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts. In certain pharmaceutical embodiments of the present disclosure a “pharmaceutically acceptable salt” may be utilized.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made. In certain pharmaceutical embodiments of the present disclosure the terms “treatment of” or “treating,” “applying,” “palliating,” or “ameliorating” may be utilized.

As used herein, “conservative substitution” means an exchange of one amino acid for another amino acid with similar properties, such as size, charge, and polarity. The substitution can be for a natural or modified (e.g., unnatural) amino acid. Non-limiting of examples, which can be interchanged in conservative substitutions, include the following groupings: Large Hydrophobics (Valine, Leucine, Isoleucine, Phenylalanine, Tryptophan, Tyrosine, Methionine), Small Non-Polar (Alanine, Glycine), Polar (Serine, Threonine, Glutamine, Asparagine, Cysteine, histidine), Positively Charged (Lysine, Arginine), and Negatively Charged (Glutamate, Aspartate).

When a % is used herein to refer to an amount of a component, unless otherwise specified, it is intended that the % be the % w/w.

The term “penetration enhancing agents” and “penetration enhancers” are used herein interchangeably. As used herein, it refers to one or more ingredients which facilitate or increase the penetration of one or more active ingredients (e.g., anionic polymeric materials such as hyaluronic acid or peptide antagonists) through one or more layers of the skin of a subject. In some embodiments, the penetration enhancing agent is a surfactant, including, for example, non-ionic surfactants having a hydrophilic-lipophilic balance (HLB) of about 10 or less, a cationic group, or another agent such as a terpene, alkaloid, salicylate derivative, nicotinate derivative, or any combination thereof.

The term “multisome” as used herein refers lipid vesicle (such as a biphasic lipid vesicle) which comprises one or more penetration enhancers, which in preferred embodiments include multiple penetration enhancers which work in a synergistic fashion. In some embodiments, multisomes include vesicle whose central core compartments are occupied by an oil-in-water emulsion composed of an aqueous continuous phase and a dispersed hydrophobic, hydrophilic or oil phase. In an embodiment, the spaces between adjacent bilayers of lipid vesicles may also be occupied by the emulsion.

The term “lipid vesicle composition” as used herein refers to a composition which includes one or more lipid vesicles (e.g., multisomal lipid vesicles, lipid bilayer vesicles, etc.). When a lipid vesicle composition is described as “comprising” one or more additional components (e.g., an anionic polymer material or a peptide antagonist provided herein), it is intended that the composition includes the additional component in any manner within the composition (e.g., encapsulated within a lipid vesicle. For example, a lipid vesicle composition comprising an anionic polymer material can include the anionic polymer material encapsulated within a lipid bilayer of the lipid vesicle composition.

The term “emulsion” as used herein refers to a mixture of two immiscible substances.

The term “bilayer” as used herein refers to a structure composed of amphiphilic lipid molecules arranged in two molecular layers, with the hydrophobic tails on the interior and the polar head groups on the exterior surfaces.

The term “topical administration” or “topical delivery” as used herein means intradermal, transdermal and/or transmucosal delivery of a compound by administration of a composition comprising the compound or compounds to skin and/or a mucosal membrane.

The term “gemini surfactant” as used herein refers to a surfactant molecule which contains more than one hydrophobic tail, and each hydrophobic tail having a hydrophilic head wherein the hydrophobic tails or hydrophilic heads are linked together by a spacer moiety. The hydrophobic tails can be identical or differ. Likewise, the hydrophilic heads can be identical or differ. The hydrophilic heads may be anionic, cationic, or neutral.

The term “HLB” or “Hydrophilic-Lipophilic Balance” value refers to standard HLB according to Griffin, J. Soc. Cosm. Chem., vol. 5, 249 (1954), which indicates the degrees of hydrophilicity and lipophilicity of a surfactant.

### Lipid Vesicle Compositions of Anionic Polymer Materials Such as Hyaluronic Acid for Intradermal Delivery

In one aspect, provided herein, is a lipid vesicle composition comprising an anionic polymer material. In some embodiments, the lipid vesicle composition comprises lipid vesicles each comprising a lipid bilayer comprising vesicle forming lipids. In some embodiments, the lipid vesicle composition comprises an oil-in-water emulsion entrapped in the lipid vesicles. In some embodiments, the oil-in-water emulsion is stabilized by one or more surfactants. In some embodiments, the anionic polymer material is entrapped in the lipid bilayer and/or the oil-in-water emulsion. In some embodiments, the anionic polymer material is entrapped within the lipid bilayer. In some embodiments, the anionic polymer material is entrapped in the oil-in-water emulsion.

### Anionic Polymer Materials

In some aspects, the lipid vesicle compositions provided herein comprise an anionic polymer material. The anionic polymer material is desirably one which is compatible with delivery beneath the surface of the skin of a subject. In some embodiments, the anionic polymer material is one which acts as a volumizer or filler after delivery beneath the surface of the skin. In some embodiments, the anionic polymer material acts as a support for another layer of skin (e.g., the epidermis) in order to correct depressions of the skin or restore facial volume.

In some embodiments, the anionic polymer material comprises an anionic polysaccharide. In some embodiments, the anionic polysaccharide is non-sulfated glycosaminoglycan. In some embodiments, the anionic polymeric material is a naturally occurring substance. In some embodiments, the anionic polymeric material naturally occurs in a human. In some embodiments, the anionic polymer material naturally occurs in connective or epithelial tissue in a human. In some embodiments, the anionic polymeric material is hyaluronic acid, or a pharmaceutically acceptable salt thereof. In some embodiments, the anionic polymer material may not be crosslinked in the lipid vesicle composition as described herein.

In some embodiments, the hyaluronic acid is a pharmaceutically acceptable salt of hyaluronic acid. In some embodiments, the salt is the sodium salt, the potassium salt, the magnesium salt, or any combination thereof. In some embodiments, the salt is the sodium salt.

In some embodiments, the anionic polymer material has a molecular weight of from about 5 kDa to about 500 kDa. In some embodiments, the molecular weight is the weight average molecular weight. In some embodiments, the anionic polymeric material has a molecular weight of about 5 kDa to about 500 kDa. In some embodiments, the anionic polymeric material has a molecular weight of about 5 kDa to about 10 kDa, about 5 kDa to about 20 kDa, about 5 kDa to about 50 kDa, about 5 kDa to about 100 kDa, about 5 kDa to about 200 kDa, about 5 kDa to about 250 kDa, about 5 kDa to about 300 kDa, about 5 kDa to about 400 kDa, about 5 kDa to about 500 kDa, about 10 kDa to about 20 kDa, about 10 kDa to about 50 kDa, about 10 kDa to about 100 kDa, about 10 kDa to about 200 kDa, about 10 kDa to about 250 kDa, about 10 kDa to about 300 kDa, about 10 kDa to about 400 kDa, about 10 kDa to about 500 kDa, about 20 kDa to about 50 kDa, about 20 kDa to about 100 kDa, about 20 kDa to about 200 kDa, about 20 kDa to about 250 kDa, about 20 kDa to about 300 kDa, about 20 kDa to about 400 kDa, about 20 kDa to about 500 kDa, about 50 kDa to about 100 kDa, about 50 kDa to about 200 kDa, about 50 kDa to about 250 kDa, about 50 kDa to about 300 kDa, about 50 kDa to about 400 kDa, about 50 kDa to about 500 kDa, about 100 kDa to about 200 kDa, about 100 kDa to about 250 kDa, about 100 kDa to about 300 kDa, about 100 kDa to about 400 kDa, about 100 kDa to about 500 kDa, about 200 kDa to about 250 kDa, about 200 kDa to about 300 kDa, about 200 kDa to about 400 kDa, about 200 kDa to about 500 kDa, about 250 kDa to about 300 kDa, about 250 kDa to about 400 kDa, about 250 kDa to about 500 kDa, about 300 kDa to about 400 kDa, about 300 kDa to about 500 kDa, or about 400 kDa to about 500 kDa. In some embodiments, the anionic polymeric material has a molecular weight of about 5 kDa, about 10 kDa, about 20 kDa, about 50 kDa, about 100 kDa, about 200 kDa, about 250 kDa, about 300 kDa, about 400 kDa, or about 500 kDa. In some embodiments, the anionic polymeric material has a molecular weight of at least about 5 kDa, about 10 kDa, about 20 kDa, about 50 kDa, about 100 kDa, about 200 kDa, about 250 kDa, about 300 kDa, or about 400 kDa. In some embodiments, the anionic polymeric material has a molecular weight of at most about 10 kDa, about 20 kDa, about 50 kDa, about 100 kDa, about 200 kDa, about 250 kDa, about 300 kDa, about 400 kDa, or about 500 kDa.

In some embodiments, the lipid vesicle composition comprises a first and a second anionic polymer material. In some embodiments, the lipid vesicle composition further comprises a third anionic polymer material.

In some embodiments, the first and the second anionic polymer material are the same type. In some embodiments, each of the first and the second anionic polymer material is an anionic polysaccharide. In some embodiments, each of the first and the second anionic polymer is hyaluronic acid.

In cases where the first and second anionic polymer materials are the same type, each anionic polymer material has a different molecular weight. In some embodiments, the first anionic polymer material has a molecular weight of up to about 75 kDa and the second anionic polymer material has a molecule weight of greater than about 75 kDa. In some embodiments, the first anionic polymer material has a molecular weight of up to about 75 kDa and the second anionic polymer material has a molecular weight of greater than about 75 kDa.

In cases where the lipid vesicle comprises a first and second anionic polymer material, each component may be included in a different amount. In some embodiments, the first and second anionic polymer material are present in about the same amount. In some embodiments, the ratio of the first and the second anionic material is about 10:1, 9:1, 8:1, 7:1, 6:1, 5:1, 4:1, 3:1, 3:2, 2:1, or about 1:1.

In some embodiments, wherein the composition comprises a first, second, and third anionic polymer material, each of the anionic polymer materials can be of the same type (e.g., three different molecular weights of hyaluronic acid). In some embodiments, the composition comprises a first, second, and a third anionic polymer material, wherein the first anionic polymer material has a molecular weight of from about 5 kDa to about 20 kDa, the second anionic polymer has a molecular weight of from about 20 kDa to about 75 kDa, and the third anionic polymer material has a molecular weight of greater than about 75 kDa. In some embodiments, each of the three anionic polymer materials is present in about the same amount.

In some embodiments, the anionic polymer material is present in an amount of from about 0.01 mg/mL to about 10 mg/mL. In some embodiments, the anionic polymer material is present in an amount of about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 1.25 mg/mL, about 0.01 mg/mL to about 1.5 mg/mL, about 0.01 mg/mL to about 1.75 mg/mL, about 0.01 mg/mL to about 2 mg/mL, about 0.01 mg/mL to about 5 mg/mL, about 0.01 mg/mL to about 10 mg/mL, about 0.05 mg/mL to about 0.1 mg/mL, about 0.05 mg/mL to about 0.5 mg/mL, about 0.05 mg/mL to about 1 mg/mL, about 0.05 mg/mL to about 1.25 mg/mL, about 0.05 mg/mL to about 1.5 mg/mL, about 0.05 mg/mL to about 1.75 mg/mL, about 0.05 mg/mL to about 2 mg/mL, about 0.05 mg/mL to about 5 mg/mL, about 0.05 mg/mL to about 10 mg/mL, about 0.1 mg/mL to about 0.5 mg/mL, about 0.1 mg/mL to about 1 mg/mL, about 0.1 mg/mL to about 1.25 mg/mL, about 0.1 mg/mL to about 1.5 mg/mL, about 0.1 mg/mL to about 1.75 mg/mL, about 0.1 mg/mL to about 2 mg/mL, about 0.1 mg/mL to about 5 mg/mL, about 0.1 mg/mL to about 10 mg/mL, about 0.5 mg/mL to about 1 mg/mL, about 0.5 mg/mL to about 1.25 mg/mL, about 0.5 mg/mL to about 1.5 mg/mL, about 0.5 mg/mL to about 1.75 mg/mL, about 0.5 mg/mL to about 2 mg/mL, about 0.5 mg/mL to about 5 mg/mL, about 0.5 mg/mL to about 10 mg/mL, about 1 mg/mL to about 1.25 mg/mL, about 1 mg/mL to about 1.5 mg/mL, about 1 mg/mL to about 1.75 mg/mL, about 1 mg/mL to about 2 mg/mL, about 1 mg/mL to about 5 mg/mL, about 1 mg/mL to about 10 mg/mL, about 1.25 mg/mL to about 1.5 mg/mL, about 1.25 mg/mL to about 1.75 mg/mL, about 1.25 mg/mL to about 2 mg/mL, about 1.25 mg/mL to about 5 mg/mL, about 1.25 mg/mL to about 10 mg/mL, about 1.5 mg/mL to about 1.75 mg/mL, about 1.5 mg/mL to about 2 mg/mL, about 1.5 mg/mL to about 5 mg/mL, about 1.5 mg/mL to about 10 mg/mL, about 1.75 mg/mL to about 2 mg/mL, about 1.75 mg/mL to about 5 mg/mL, about 1.75 mg/mL to about 10 mg/mL, about 2 mg/mL to about 5 mg/mL, about 2 mg/mL to about 10 mg/mL, or about 5 mg/mL to about 10 mg/mL. In some embodiments, the anionic polymer material is present in an amount of about 0.01 mg/mL, about 0.05 mg/mL, about 0.1 mg/mL, about 0.5 mg/mL, about 1 mg/mL, about 1.25 mg/mL, about 1.5 mg/mL, about 1.75 mg/mL, about 2 mg/mL, about 5 mg/mL, or about 10 mg/mL. In some embodiments, the anionic polymer material is present in an amount of at least about 0.01 mg/mL, about 0.05 mg/mL, about 0.1 mg/mL, about 0.5 mg/mL, about 1 mg/mL, about 1.25 mg/mL, about 1.5 mg/mL, about 1.75 mg/mL, about 2 mg/mL, or about 5 mg/mL. In some embodiments, the anionic polymer material is present in an amount of at most about 0.05 mg/mL, about 0.1 mg/mL, about 0.5 mg/mL, about 1 mg/mL, about 1.25 mg/mL, about 1.5 mg/mL, about 1.75 mg/mL, about 2 mg/mL, about 5 mg/mL, or about 10 mg/mL.

### Vesicle Forming Lipids

In some embodiments, the vesicle composition comprises one or more vesicle forming lipids. The vesicle forming lipids act to encapsulate portions of the oil-in-water emulsions. In some embodiments, this allows the oil-in-water emulsion to remain stable for a period of time.

The vesicle forming lipids may be any suitable lipids for such a purpose. In some embodiments, the vesicle forming lipids comprise phospholipids, glycolipids, lecithins, ceramides, lysolecithin, lysophosphatidylethanolamine, phosphatidylserine, phosphatidylinositol, sphingomyelin, cardiolipin, phosphatidic acid, cerebroside, or any combination thereof. In some embodiments, the vesicle forming lipids comprise a combination of lipids.

In some embodiments, the vesicle forming lipids comprise phospholipids. In some embodiments, the phospholipids are naturally occurring, semisynthetic, or synthetically prepared, or a mixture thereof. In an embodiment, the phospholipids are one or more esters of glycerol with one or two (equal or different) residues of fatty adds and with phosphoric acid, wherein the phosphoric acid residue is in turn bound to a hydrophilic group, such as, for instance, choline (phosphatidylcholines—PC), serine (phosphatidylserines—PS), glycerol (phosphatidylglycerols—PG), ethanolamine (phosphatidylethanolamines—PE), or inositol (phosphatidylinositol). Esters of phospholipids with only one residue of fatty acid are generally referred to in the art as the “lyso” forms of the phospholipid or “lysophospholipids”. Fatty acids residues present in the phospholipids are in general long chain aliphatic acids, typically containing 12 to 24 carbon atoms, or 14 to 22 carbon atoms; the aliphatic chain may contain one or more unsaturations or is completely saturated. Examples of suitable fatty acids included in the phospholipids are, for instance, lauric acid, myristic acid, palmitic acid, stearic acid, arachidic acid, behenic acid, oleic acid, linoleic acid, and linolenic acid. Saturated fatty acids such as myristic acid, palmitic acid, stearic acid and arachidic acid may be employed.

In some embodiments, the phospholipid comprises one or more natural phospholipids. In some embodiments, the phospholipid comprises one or more semisynthetic phospholipids. In some embodiments, the semisynthetic phospholipids are the partially or fully hydrogenated derivatives of the naturally occurring lecithins. In some embodiments, the phospholipids include fatty acids di-esters of phosphatidylcholine, ethylphosphatidylcholine, phosphatidylglycerol, phosphatidic acid, phosphatidylethanolamine, phosphatidylserine or of sphingomyelin. In some embodiments, the phospholipids include hydrogenated phosphatidylcholine (e.g., Sunlipon 90H). In some embodiments, the phospholipids are, for instance, dilauroyl-phosphatidylcholine (DLPC), dimyristoyl-phosphatidylcholine (DMPC), dipalmitoyl-phosphatidylcholine (DPPC), diarachidoyl-phosphatidylcholine (DAPC), distearoyl-phosphatidylcholine (DSPC), dioleoyl-phosphatidylcholine (DOPC), 1,2Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), 1-palmitoyl-2-oleylphosphatidylcholine (POPC), 1-oleyl-2-palmitoyl-phosphatidylcholine (OPPC), dilauroylphosphatidylglycerol (DLPG) and its alkali metal salts, diarachidoylphosphatidylglycerol (DAPG) and its alkali metal salts, dimyristoylphosphatidylglycerol (DMPG) and its alkali metal salts, dipalmitoylphosphatidylglycerol (DPPG) and its alkali metal salts, distearoylphosphatidylglycerol (DSPG) and its alkali metal salts, dioleoyl-phosphatidylglycerol (DOPG) and its alkali metal salts, dimyristoyl phosphatidic acid DMPA) and its alkali metal salts, dipalmitoyl phosphatidic acid (DPPA) and its alkali metal salts, distearoyl phosphatidic acid (DSPA), diarachidoylphosphatidic acid (DAPA) and its alkali metal salts, dimyristoylphosphatidylethanolamine (DMPE), dipalmitoylphosphatidylethanolamine (DPPE), distearoyl phosphatidyl-ethanolamine (DSPE), dioleylphosphatidylethanolamine (DOPE), diarachidoylphosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), and distearoylsphingomyelin (DSSP), dilauroyl-phosphatidylinositol (DLPI), diarachidoylphosphatidylinositol (DAPI), dimyristoylphosphatidylinositol (DMPI), dipalmitoylphosphatidylinositol (DPPI), distearoylphosphatidylinositol (DSPI), dioleoyl-phosphatidylinositol (DOPI).

In some embodiments, the vesicle forming lipids are present in an amount of about 0.5% to about 25% (w/w) of the composition. In some embodiments, the vesicle forming lipids are present in an amount of about 0.5% to about 2%, about 0.5% to about 5%, about 0.5% to about 8%, about 0.5% to about 10%, about 0.5% to about 12%, about 0.5% to about 15%, about 0.5% to about 20%, about 0.5% to about 25%, about 2% to about 5%, about 2% to about 8%, about 2% to about 10%, about 2% to about 12%, about 2% to about 15%, about 2% to about 20%, about 2% to about 25%, about 5% to about 8%, about 5% to about 10%, about 5% to about 12%, about 5% to about 15%, about 5% to about 20%, about 5% to about 25%, about 8% to about 10%, about 8% to about 12%, about 8% to about 15%, about 8% to about 20%, about 8% to about 25%, about 10% to about 12%, about 10% to about 15%, about 10% to about 20%, about 10% to about 25%, about 12% to about 15%, about 12% to about 20%, about 12% to about 25%, about 15% to about 20%, about 15% to about 25%, or about 20% to about 25% (w/w) of the composition. In some embodiments, the vesicle forming lipids are present in an amount of about 0.5%, about 2%, about 5%, about 8%, about 10%, about 12%, about 15%, about 20%, or about 25%. In some embodiments, the vesicle forming lipids are present in an amount of at least about 0.5%, about 2%, about 5%, about 8%, about 10%, about 12%, about 15%, or about 20% (w/w) of the composition. In some embodiments, the vesicle forming lipids are present in an amount of at most about 2%, about 5%, about 8% about 10%, about 12%, about 15%, about 20%, or about 25% (w/w) of the composition.

In some embodiments, the vesicle forming lipids are present in an amount of about 5% to about 15% (w/w) of the composition. In some embodiments, the vesicle forming lipids are present in an amount of about 5% to about 8%, about 5% to about 9%, about 5% to about 10%, about 5% to about 11%, about 5% to about 12%, about 5% to about 13%, about 5% to about 14%, about 5% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 11% to about 12%, about 11% to about 13%, about 11% to about 14%, about 11% to about 15%, about 12% to about 13%, about 12% to about 14%, about 12% to about 15%, about 13% to about 14%, about 13% to about 15%, or about 14% to about 15%. In some embodiments, the vesicle forming lipids are present in an amount of about 5%, about 8%, about 9%, about 10%, about 11%, about 12%, about 13%, about 14% or about 15% (w/w) of the composition. In some embodiments, the vesicle forming lipids are present in an amount of at least about 5%, about 8%, about 9%, about 10%, about 11%, about 12%, about 13%, or about 14% (w/w) of the composition. In some embodiments, the vesicle forming lipids are present in an amount of at most about 8%, about 9%, about 10%, about 11%, about 12%, about 13%, about 14%, or about 15% (w/w) of the composition.

In some embodiments, the composition comprises a short chain polyol. In some embodiments, the short chain polyol acts to enhance the stability of the resulting lipid vesicles. In some embodiments, the short chain polyol is a C₂-C₄ polyol comprising two or three alcohol groups. In some embodiments, the short chain polyol is propylene glycol. In some embodiments, the composition comprises propylene glycol.

In some embodiments, the propylene glycol is present in an amount of about 0.5% to about 25% (w/w) of the composition. In some embodiments, the propylene glycol is present in an amount of about 0.5% to about 2%, about 0.5% to about 5%, about 0.5% to about 8%, about 0.5% to about 10%, about 0.5% to about 12%, about 0.5% to about 15%, about 0.5% to about 20%, about 0.5% to about 25%, about 2% to about 5%, about 2% to about 8%, about 2% to about 10%, about 2% to about 12%, about 2% to about 15%, about 2% to about 20%, about 2% to about 25%, about 5% to about 8%, about 5% to about 10%, about 5% to about 12%, about 5% to about 15%, about 5% to about 20%, about 5% to about 25%, about 8% to about 10%, about 8% to about 12%, about 8% to about 15%, about 8% to about 20% about 8% to about 25%, about 10% to about 12%, about 10% to about 15%, about 10% to about 20%, about 10% to about 25%, about 12% to about 15%, about 12% to about 20%, about 12% to about 25%, about 15% to about 20%, about 15% to about 25%, or about 20% to about 25%. In some embodiments, the propylene glycol is present in an amount of about 0.5%, about 2% about 5%, about 8%, about 10%, about 12%, about 15%, about 20%, or about 25%. In some embodiments, the propylene glycol is present in an amount of at least about 0.5%, about 2%, about 5%, about 8%, about 10%, about 12%, about 15%, or about 20%. In some embodiments, the propylene glycol is present in an amount of at most about 2%, about 5% about 8%, about 10%, about 12%, about 15%, about 20%, or about 25%. In some embodiments, the propylene glycol is present in an amount of about 1% to about 10%. In some embodiments, the propylene glycol is present in an amount of about 1% to about 2%, about 1% to about 4%, about 1% to about 6%, about 1% to about 8%, about 1% to about 10%, about 2% to about 4%, about 2% to about 6%, about 2% to about 8%, about 2% to about 10%, about 4% to about 6%, about 4% to about 8%, about 4% to about 10%, about 6% to about 8%, about 6% to about 10%, or about 8% to about 10%. In some embodiments, the propylene glycol is present in an amount of about 1%, about 2%, about 4%, about 6%, about 8%, or about 10%. In some embodiments, the propylene glycol is present in an amount of at least about 1%, about 2%, about 4%, about 6%, or about 8%. In some embodiments, the propylene glycol is present in an amount of at most about 2%, about 4%, about 6%, about 8%, or about 10%. In some embodiments, propylene glycol is present in about the same amount as the vesicle forming lipid. In some embodiments, the ratio of propylene glycol to vesicle forming lipid in the composition is form about 2:1 to about 1:2 (w/w). In some embodiments, the ratio of propylene glycol to vesicle forming lipid in the composition is form about 1:1 (w/w).

### Oil Phases

The lipid vesicle compositions provided herein comprise an oil-in-water emulsion. The oil component is selected such that the material is a liquid at operative temperatures (e.g., room temperature) and is non-miscible with water.

Any suitable oil may be used as the oil phase. In some embodiments, the oil comprises a naturally occurring oil. In some embodiments, the naturally occurring oil is derived from one or more plants or plant parts (e.g., seeds or nuts). In some embodiments, the oil is a naturally occurring oil such as olive oil, vegetable oil, sunflower oil, or other similar plant derived oil.

In some embodiments, the oil phase is selected from the group consisting of vegetable oils, mono-, di-, and triglycerides, silicone fluids, mineral oils, and combinations thereof. In some embodiments, the oil phase comprises an emollient. In some embodiments, the emollient comprises caprylic and/or capric triglycerides (e.g., Labrafac CC). IN some embodiments, the emollient comprises a natural oil-soluble emollient, such an oil-soluble plant extracts, essential oils, vegetable oils, vegetable butters, or any combination thereof. In some embodiments, the natural oil-soluble emollient comprise oils from sunflowers. In some embodiments, the natural oil-soluble emollient comprise oils from avocados.

In some embodiments, the oil comprises a silicon oil or derivative, such as dimethicone. In some embodiments, the silicon oil comprises a siloxane polymer. In some embodiments, the siloxane polymer comprises C₁-C₃ substituents. In some embodiments, the siloxane is polydimethylsiloxane (PDMS). In some embodiments, the oil is a mixture which comprises a silicon oil (e.g., dimethicone) as a smaller component. In some embodiments, the oil is a mixture which comprises a natural emollient as a substitute for dimethicone (e.g., LexFeel™ N350 MB). In some embodiments, the silicon oil is incorporated in order to enhance the feel of the resulting composition or as a moisturizer. In some embodiments, the oil comprises a silicon oil in an amount of up to about 5%, up to about 4%, up to about 3%, up to about 2%, or up to about 1% (w/w) of the composition. In some embodiments, the silicon oil is present in an amount of from about 0.1% to about 2%.

In some embodiments, the oils are present in an amount of about 1% to about 35% (w/w) of the composition. In some embodiments, the oils are present in an amount of about 1% to about 5%, about 1% to about 10%, about 1% to about 15%, about 1% to about 20%, about 1% to about 25%, about 1% to about 30%, about 1% to about 35%, about 5% to about 1000, about 5% to about 15%, about 5% to about 20%, about 5% to about 25%, about 5% to about 30%, about 5% to about 35%, about 10% to about 15%, about 10% to about 20%, about 10% to about 25%, about 10% to about 30%, about 10% to about 35%, about 15% to about 20%, about 15% to about 25%, about 15% to about 30%, about 15% to about 35%, about 20% to about 25%, about 20% to about 30%, about 20% to about 35%, about 25% to about 30% about 25% to about 35%, or about 30% to about 35%. In some embodiments, the oils are present in an amount of about 1%, about 5%, about 10%, about 15%, about 20%, about 25%, about 30%, or about 35%. In some embodiments, the oils are present in an amount of at least about 1%, about 5%, about 10%, about 15%, about 20%, about 25%, or about 30%. In some embodiments, the oils are present in an amount of at most about 5%, about 10%, about 15%, about 20%, about 25%, about 30%, or about 35%. In some embodiments, the oils are present in an amount of about 5% to about 15%. In some embodiments, the oils are present in an amount of about 5% to about 8%, about 5% to about 9%, about 5% to about 10%, about 5% to about 11%, about 5% to about 12%, about 5% to about 13%, about 5% to about 14%, about 5% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 11% to about 12%, about 11% to about 13%, about 11% to about 14%, about 11% to about 15%, about 12% to about 13%, about 12% to about 14%, about 12% to about 15%, about 13% to about 14%, about 13% to about 15%, or about 14% to about 15%. In some embodiments, the oils are present in an amount of about 5%, about 8%, about 9%, about 10%, about 11%, about 12%, about 13%, about 14%, or about 15%. In some embodiments, the oils are present in an amount of at least about 5%, about 8%, about 9%, about 1000, about 11%, about 12%, about 13%, or about 14%. In some embodiments, the oils are present in an amount of at most about 8%, about 9%, about 10%, about 11%, about 12% about 13%, about 14%, or about 15%.

In some embodiments, the oil comprises one or more triglycerides. In some embodiments the triglyceride is a medium chain triglyceride. In some embodiments, the medium chain triglyceride comprises fatty acid esters having a chain length of C₆-C₁₂.

In some embodiments, the triglyceride is present in an amount of about 1% to about 35% (w/w) of the composition. In some embodiments, the triglyceride is present in an amount of about 1% to about 5%, about 1% to about 10%, about 1% to about 15%, about 1% to about 20%, about 1% to about 25%, about 1% to about 30%, about 1% to about 35%, about 5% to about 10%, about 5% to about 15%, about 5% to about 20%, about 5% to about 25%, about 5% to about 30%, about 5% to about 35%, about 10% to about 15%, about 10% to about 20%, about 10% to about 25%, about 10% to about 30%, about 10% to about 35%, about 15% to about 20%, about 15% to about 25%, about 15% to about 30%, about 15% to about 35%, about 20% to about 25%, about 20% to about 30%, about 20% to about 35%, about 25% to about 30%, about 25% to about 35%, or about 30% to about 35%. In some embodiments, the triglyceride is present in an amount of about 1%, about 5%, about 10%, about 15%, about 20%, about 25%, about 30%, or about 35%. In some embodiments, the triglyceride is present in an amount of at least about 1%, about 5%, about 10%, about 15%, about 20%, about 25%, or about 30%. In some embodiments, the triglyceride is present in an amount of at most about 5% about 10% about 15%, about 20%, about 25%, about 30%, or about 35%.

In some embodiments, the oil phase of the lipid vesicle and/or the lipid vesicle portion of the composition comprises a sterol. In some embodiments, the sterol is cholesterol. In some embodiments, the cholesterol may be plant-derived cholesterol. In some embodiments, the plant-derived cholesterol may be PhytoChol®, SyntheChol®, or any other plant-derived cholesterol (e.g., Avanti #700100), or any combination thereof. In some embodiments, the sterol may be phytosterol or a derivative thereof. In some embodiments, the phytosterol or derivative thereof may be phytosterol MM, Advasterol™ 90 IP or 95 IP F, NET Sterol-ISO, canola sterols, sitosterol 700095, lanosterol-95, brassicasterol, or any combination thereof.

In some embodiments, the sterol is present in an amount of about 1% to about 5% (w/w) of the composition. In some embodiments, the sterol is present in an amount of about 1% to about 1.5%, about 1% to about 2%, about 1% to about 2.5%, about 1% to about 3%, about 1% to about 4%, about 1% to about 5%, about 1.5% to about 2%, about 1.5% to about 2.5% about 1.5% to about 3%, about 1.5% to about 4%, about 1.5% to about 5%, about 2% to about 2.5%, about 2% to about 3%, about 2% to about 4%, about 2% to about 5%, about 2.5% to about 3%, about 2.5% to about 4%, about 2.5% to about 5%, about 3% to about 4%, about 3% to about 5%, or about 4% to about 5% (w/w) of the composition. In some embodiments, the sterol is present in an amount of about 1%, about 1.5%, about 2%, about 2.5%, about 3%, about 4%, or about 5% (w/w) of the composition. In some embodiments, the sterol is present in an amount of at least about 1%, about 1.5%, about 2%, about 2.5%, about 3%, or about 4% (w/w) of the composition. In some embodiments, the sterol is present in an amount of at most about 1.5%, about 2%, about 2.5%, about 3%, about 4%, or about 5% (w/w) of the composition. In some embodiments, the sterol is present in an amount of about 2.6% (w/w) of the composition. In some embodiments, the ratio of sterol to vesicle forming lipids is from about 1:2 to about 1:5 (w/w). In some embodiments, the ratio of sterol to vesicle forming lipids is about 1:2, 1:3, 1:4, or 1:5 (w/w).

### Penetration Enhancers

In some embodiments, the lipid vesicle compositions comprise one or more penetration enhancers. Penetration enhancers act to increase the amount of penetration of the anionic polymer material through one or more layers of skin when applied to the skin of an individual.

In some embodiments, the penetration enhancer is included in the oil-in-water emulsion of the composition. In some embodiments, the penetration enhancer is included in the lipid bilayer of the composition.

There are many types of penetration enhancing agents that may be employed. In some embodiments, the penetration enhancing agent comprising an ionic surfactant, a nonionic surfactant, or a combination thereof.

In some embodiments, the penetration enhancing agent comprises a non-ionic surfactant or a combination of non-ionic surfactants. In some embodiments, the penetration enhancing agent is a single non-ionic surfactant. In some embodiments, the penetration enhancing agent is a combination of at least 2, 3, 4, or more non-ionic surfactants. In some embodiments, the penetration enhancing agent is a combination 2 non-ionic surfactants. In some embodiments, the penetration enhancing agent is a combination 3 non-ionic surfactants.

In some embodiments, the non-ionic surfactant or combination of non-ionic surfactants is selected from polyethylene glycol ethers of fatty alcohols, sorbitan esters, polysorbates, sorbitan esters and polyethylene glycol fatty acid esters and combinations thereof.

In some embodiments, the non-ionic surfactant comprises a polyethylene glycol (PEG) ethers of a fatty alcohol. In some embodiments, the PEG ether of the fatty alcohol comprises from about 2 to about 8 PEG groups and a C₁₂-C₂₂ fatty alcohol. In some embodiments, the polyethylene glycol ether of a fatty alcohol comprises diethylene glycol hexadecyl ether, 2-(2-octadecoxyethoxy)ethanol, diethylene glycol monooleyl ether, polyoxyethylene (2) oleyl ether, polyoxyethylene (3) oleyl ether, or polyoxyethylene (5) oleyl ether, or any combination thereof. In some embodiments, the polyethylene glycol ether of a fatty alcohol comprises 2-(2-octadecoxyethoxy)ethanol. In some embodiments, the PEG ether of a fatty alcohol is super refined Brij® O2 or a derivative thereof.

In some embodiments, the PEG ether of the fatty alcohol is present in an amount of from about 0.5% to about 10%, about 0.5% to about 5%, about 0.5% to about 4%, or about 0.05% to about 3% (w/w) of the composition. In some embodiments, the PEG ether of the fatty alcohol is present in an amount of about 0.5% to about 2.5%. In some embodiments, the PEG ether of the fatty alcohol is present in an amount of about 0.5% to about 0.8%, about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the PEG ether of the fatty alcohol is present in an amount of about 0.5%, about 0.8%, about 1%, about 1.2%, about 1.5%, about 2%, or about 2.5%. In some embodiments, the PEG ether of the fatty alcohol is present in an amount of at least about 0.5%, about 0.8%, about 1%, about 1.2%, about 1.5%, or about 2%. In some embodiments, the PEG ether of the fatty alcohol is present in an amount of at most about 0.8%, about 1%, about 1.2%, about 1.5%, about 2%, or about 2.5%.

In some embodiments, the non-ionic surfactant comprises a sorbitan ester. In some embodiments, the sorbitan ester is a fatty acid ester. In some embodiments, the sorbitan ester is a C₁₂-C₂₂ fatty acid ester. In some embodiments, the sorbitan ester comprises sorbitan monolaurate, sorbitan monopalmitate, sorbitan monostearate, sorbitan monooleate, sorbitan trioleate, sorbitan sesquioleate, or sorbitan isostearate, or any combinations thereof. In some embodiments, the sorbitan ester comprises sorbitan monolaurate. In some embodiments, the sorbitan ester comprises sorbitan monopalmitate. In some embodiments, the sorbitan ester comprises sorbitan monostearate. In some embodiments, the sorbitan ester comprises sorbitan monooleate. In some embodiments, the sorbitan ester comprises sorbitan trioleate. In some embodiments, the sorbitan ester comprises sorbitan sesquioleate. In some embodiments, the sorbitan ester comprises sorbitan isostearate.

In some embodiments, the sorbitan ester is present in an amount of up to about 5% (w/w) of the composition. In some embodiments, the sorbitan ester is present in an amount of from about 0.1% to about 0.5%. In some embodiments, the sorbitan ester is present in an amount of from about 0.1% to about 0.15%, about 0.1% to about 0.2%, about 0.1% to about 0.25%, about 0.1% to about 0.3%, about 0.1% to about 0.35%, about 0.1% to about 0.4%, about 0.1% to about 0.45%, about 0.1% to about 0.5%, about 0.15% to about 0.2%, about 0.15% to about 0.25%, about 0.15% to about 0.3%, about 0.15% to about 0.35%, about 0.15% to about 0.4%, about 0.15% to about 0.45%, about 0.15% to about 0.5%, about 0.2% to about 0.25%, about 0.2% to about 0.3%, about 0.2% to about 0.35%, about 0.2% to about 0.4%, about 0.2% to about 0.45%, about 0.2% to about 0.5%, about 0.25% to about 0.3%, about 0.25% to about 0.35%, about 0.25% to about 0.4%, about 0.25% to about 0.45%, about 0.25% to about 0.5%, about 0.3% to about 0.35%, about 0.3% to about 0.4%, about 0.3% to about 0.45%, about 0.3% to about 0.5%, about 0.35% to about 0.4%, about 0.35% to about 0.45%, about 0.35% to about 0.5%, about 0.4% to about 0.45%, about 0.4% to about 0.5%, or about 0.45% to about 0.5%. In some embodiments, the sorbitan ester is present in an amount of from about 0.1%, about 0.15%, about 0.2%, about 0.25%, about 0.3%, about 0.35%, about 0.4%, about 0.45%, or about 0.5%. In some embodiments, the sorbitan ester is present in an amount of from at least about 0.1%, about 0.15%, about 0.2%, about 0.25%, about 0.3%, about 0.35%, about 0.4%, or about 0.45%. In some embodiments, the sorbitan ester is present in an amount of from at most about 0.15%, about 0.2%, about 0.25%, about 0.3%, about 0.35%, about 0.4%, about 0.45%, or about 0.5%.

In some embodiments, the sorbitan ester is present in an amount of from about 0.1% to about 5%, 0.2% to about 5%, 0.3% to about 5%, 0.4% to about 5%, 0.5% to about 5%, about 0.5% to about 4%, or about 0.5% to about 3%. In some embodiments, the sorbitan ester is present in an amount of about 0.5% to about 2.5%. In some embodiments, the sorbitan ester is present in an amount of about 0.5% to about 0.8%, about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the sorbitan ester is present in an amount of about 0.5%, about 0.8% about 1%, about 1.2%, about 1.5%, about 2%, or about 2.5%. In some embodiments, the sorbitan ester is present in an amount of at least about 0.5%, about 0.8% about 1%, about 1.2%, about 1.5%, or about 2%. In some embodiments, the sorbitan ester is present in an amount of at most about 0.8%, about 1%, about 1.2%, about 1.5%, about 2 00 or about 2.5%.

In some embodiments, the non-ionic surfactant comprises a polysorbate. In some embodiments, the polysorbate comprises polysorbate 20, polysorbate 21, polysorbate 40, polysorbate 60, polysorbate 80, polysorbate 85, or any combination thereof. In some embodiments, the polysorbate is polysorbate 80. In some embodiments, the polysorbate is polysorbate 20.

In some embodiments, the polysorbate is present in an amount of up to about 5%. In some embodiments, the polysorbate is present in an amount of from about 0.5% to about 5% about 0.5% to about 4%, or about 0.5% to about 3% (w/w) of the composition. In some embodiments, the polysorbate is present in an amount of about 0.5% to about 2.5%. In some embodiments, the polysorbate is present in an amount of about 0.5% to about 0.8% about 0.5% to about 1%, about 0.5% to about 1.2%, about 0.5% to about 1.5%, about 0.5% to about 2% about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2% about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the polysorbate is present in an amount of about 0.5%, about 0.8%, about 1%, about 1.2%, about 1.5%, about 2%, or about 2.5%. In some embodiments, the polysorbate is present in an amount of at least about 0.5%, about 0.8%, about 1%, about 1.2%, about 1.5%, or about 2%. In some embodiments, the polysorbate is present in an amount of at most about 0.8% about 1%, about 1.2%, about 1.5%, about 2%, or about 2.5%.

In some embodiments, the non-ionic surfactant comprises a polyethylene glycol (PEG) fatty acid ester. In some embodiments, the PEG fatty acid ester is a PEG chain of about 2-8 subunits comprising C₈-C₂₂ fatty acids affixed to each terminal hydroxyl to form the fatty acid ester. In some embodiments, the PEG fatty acid ester comprises PEG-8 dilaurate, PEG-4 dilaurate, PEG-4 laurate, PEG-8 dioleate, PEG-8 distearate, PEG-8 distearate, PEG-7 glyceryl cocoate, and PEG-20 almond glycerides, or any combination thereof. In some embodiments, the PEG fatty acid ester is PEG-4 dilaurate.

In some embodiments, the PEG fatty acid ester is present in an amount of up to about 5% (w/w) of the composition. In some embodiments, the PEG fatty acid ester is present in an amount of from about 0.5% to about 5%, about 0.5% to about 4%, or about 0.5% to about 3%. In some embodiments, the PEG fatty ester is present in an amount of about 0.5% to about 2.5%. In some embodiments, the PEG fatty ester is present in an amount of about 0.5% to about 0.8% about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the PEG fatty ester is present in an amount of about 0.5%, about 0.8% about 1%, about 1.2%, about 1.5%, about 2%, or about 2.5%. In some embodiments, the PEG fatty ester is present in an amount of at least about 0.5%, about 0.8%, about 1%, about 1.2%, about 1.5%, or about 2%. In some embodiments, the PEG fatty ester is present in an amount of at most about 0.8% about 1%, about 1.2%, about 1.5%, about 2%, or about 2.5%.

In some embodiments, the non-ionic surfactant has a hydrophobic-lipophilic balance (HLB) of about 10 or less. In some embodiments, the non-ionic surfactant comprises glyceryl monostearate (e.g., Cithrol GMS 40). In some embodiments, the non-ionic surfactant comprises an oleyl alcohol (e.g., Lipocol O-95). In some embodiments, the non-ionic surfactant comprises a polyoxyethylene oleyl ether (e.g., Oleth-2). In some embodiments, the non-ionic surfactant comprises a propylene glycol monocaprylate (e.g., Capryol® 90). In some embodiments, the composition comprises a plurality of non-ionic surfactants, each having an HLB of about 10 or less. In some embodiments, the non-ionic surfactant with an HLB of 10 or less is selected from the Table 1 below, or any combination thereof.

TABLE 1

| Category | INCI/Chemical name | Properties |
|---|---|---|
| | Ce [symbol] -2 [symbol] (Diethylene glycol hex [symbol] ether) | HLB = 5.3 |
| | Steareth-2 [symbol] (2-(2- [symbol] oxy)ethanol) | HLB = 4.9 |
| | O [symbol] th-2 [symbol] (Polyoxyethylene (2) Oleyl Ether/ | HLB = 4.9 |
| | Diethylene glycol m [symbol] ether) | |
| | O [symbol] th-3 [symbol] (Polyoxyethylene (3) Oleyl Ether) | HLB = 6.6 |
| | O [symbol] th-5 [symbol] (Polyoxyethylene (5) Oleyl Ether) | HLB = [symbol] |
| | Polysorbate [symbol] | HLB = 9. [symbol] |
| | Sorbitan mono [symbol] | HLB = [symbol] . [symbol] |
| | Sorbitan monopalmitate | HLB = [symbol] .7 |
| | Sorbitan monostearate | HLB = 4.7 |
| | Sorbitan mono [symbol] | HLB = 4.3 |
| | Sorbitan [symbol] | HLB = 1.8 |
| | Sorbitan [symbol] | HLB = 3.7 |
| | Sorbitan Isostearate | HLB = 4.7 |
| | PEG- [symbol] | HLB = 10 |
| | PEG-4 d [symbol] (Polyoxyethylene ( [symbol] ) [symbol] aurate) | HLB = [symbol] |
| | PEG-4 laurate ((Polyoxyethylene (4) | HLB = [symbol] |
| | d [symbol] urate) | |
| | PEG- [symbol] | HLB = 7.2 |
| | PEG- [symbol] rate | HLB = [symbol] |
| | PEG-7 [symbol] | HLB = 10 |
| | PEG-20 almond [symbol] | HLB = 10 |
| | Propylene glycol [symbol] | HLB = 2.5 |
| | Gly [symbol] stearate | HLB = 2. [symbol] |
| | Gly [symbol] | HLB = 3.8 |
| | Gly [symbol] stearate SE | HLB = [symbol] .8 |
| | Gly [symbol] | HLB = 5.2 |
| | Gly [symbol] caprylat [symbol] | HLB = 5 [symbol] 6 |
| | PEG-30 dipoly [symbol] | HLB = 5.5 |
| | Gly [symbol] | HLB = 1 [symbol] and |
| | Phosph [symbol] | HLB [symbol] 10 |

[symbol]

[symbol]

indicates data missing or illegible when filed

In some embodiments, the non-ionic surfactant has a hydrophobic-lipophilic balance (HLB) of about 10 or more. In some embodiments, the composition comprises a plurality of non-ionic surfactants, each having an HLB of about 10 or more.

In some embodiments, the non-ionic surfactant or combination of non-ionic surfactants are present in an amount of about 0.5% to about 10% (w/w) of the composition. In some embodiments, the non-ionic surfactant or combination of non-ionic surfactants are present in an amount of about 0.5% to about 1%, about 0.5% to about 1.5%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 10%, about 1% to about 1.5%, about 1% to about 2%, about 1% to about 3%, about 1% to about 4%, about 1% to about 5%, about 1% to about 6%, about 1% to about 7%, about 1% to about 8%, about 1% to about 10%, about 1.5% to about 2%, about 1.5% to about 3%, about 1.5% to about 4%, about 1.5% to about 5%, about 1.5% to about 6%, about 1.5% to about 7%, about 1.5% to about 8%, about 1.5% to about 10%, about 2% to about 3%, about 2% to about 4%, about 2% to about 5%, about 2% to about 6%, about 2% to about 7%, about 2% to about 8%, about 2% to about 10%, about 3% to about 4%, about 3% to about 5%, about 3% to about 6%, about 3% to about 7%, about 3% to about 8%, about 3% to about 10% about 4% to about 5%, about 4% to about 6%, about 4% to about 7%, about 4% to about 8% about 4% to about 10%, about 5% to about 6%, about 5% to about 7%, about 5% to about 8%, about 5% to about 10%, about 6% to about 7%, about 6% to about 8%, about 6% to about 10%, about 7% to about 8%, about 7% to about 10%, or about 8% to about 10%. In some embodiments, the non-ionic surfactant or combination of non-ionic surfactants are present in an amount of about 0.5%, about 1%, about 1.5% about 2%, about 3%, about 4%, about 5%, about 6% about 7%, about 8% or about 10%. In some embodiments, the non-ionic surfactant is present in an amount of 1.4%. In some embodiments, the non-ionic surfactant or combination of non-ionic surfactants are present in an amount of at least about 0.5%, about 1%, about 1.5% about 2%, about 3%, about 4%, about 5%, about 6%, about 7%, or about 8%. In some embodiments, the non-ionic surfactant or combination of non-ionic surfactants are present in an amount of at most about 1%, about 1.5%, about 2%, about 3%, about 4%, about 5%, about 6%, about 7%, about 8%, or about 10%.

In some embodiments, the composition comprises a non-ionic surfactant in the oil-in-water emulsion, the lipid bilayer, or both. In some embodiments, the composition comprises a non-ionic surfactant in the oil-in-water emulsion. In some embodiments, the composition comprises a non-ionic surfactant in the lipid bilayer. In some embodiments, the composition comprises a non-ionic surfactant in the oil-in-water emulsion and the lipid bilayer, wherein the composition comprises two or more different non-ionic surfactants.

In some embodiments, the penetration enhancing agent comprises a salicylate ester or a nicotinate ester. In some embodiments, the ester is a C₁-C₆ alkyl ester or a benzyl ester. In some embodiments, the penetration enhancing agent comprises methyl salicylate or benzyl nicotinate. In some embodiments, the penetration enhancing agent is a nicotinate ester present in an amount of up to about 0.1%, 0.5%, 1%, 2%, or 3% (w/w) of the composition. In some embodiments, the nicotinate ester is present in an amount of from about 0.1% to about 3%, about 0.1% to about 2%, or about 0.10% to about 1%.

### Cationic Surfactants

In some embodiments, the composition comprises an ionic surfactant. In some embodiments, the ionic surfactant is a cationic surfactant. In some embodiments, the cationic surfactant is a mono-cationic surfactant, a di-cationic surfactant, or a poly-cationic surfactant.

In some embodiments, the mono-cationic surfactant is used in the composition to form a submicron emulsion prior to formation of a final lipid vesicle composition provided herein (e.g., before the lipid forming vesicles are added). In some embodiments, the mono-cationic surfactant is net-mono-cationic (e.g., a phosphate salt comprising two side chains each with a single cationic functionality, which is partially neutralized by a phosphate anion).

In some embodiments, the mono-cationic surfactant is a fatty-amide derived propylene glycol-diammonium phosphate ester. Fatty-amide derived propylene glycol-diammonium phosphate esters are phospholipids which comprise at least one propylene glycol phosphoester linked to a quaternary ammonium group, which is in turn linked with a fatty acid amide. One non-limiting example of a fatty-amide derived propylene glycol-diammonium phosphate ester is linoleamidopropyl PG-dimonium chloride phosphate. Similar compounds with different fatty acid amide groups attached are also known. In some embodiments, the fatty-amide derived propylene glycol-diammoniom phosphate ester has the structure:

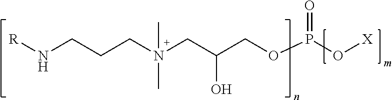

wherein n is an integer from 1 to 3, m is an integer from 0 to 2, wherein the sum of m and n is 3; X is a cation selected from a proton, sodium, potassium, magnesium, and calcium; and R is an acyl group of a C₈-C₃₀ fatty acid.

In some embodiments, the fatty acid is a C₁₂-C₂₄ fatty acid. In some embodiments, the fatty acid is an unsaturated fatty acid. In some embodiments, the fatty acid is linoleic acid. In some embodiments, the mono-cationic penetration enhancing agent is linoleamidopropyl PG-dimonium chloride phosphate (e.g., Arlasilk™ PTM, Arlasilk™ EFA).

In some embodiments, the fatty amide derived propylene glycol-diammonium phosphate ester is present in an amount of about 1% to about 10% (w/w) of the composition. In some embodiments, the fatty amide derived propylene glycol-diammonium phosphate ester is present in an amount of about 1% to about 2%, about 1% to about 3%, about 1% to about 4%, about 1% to about 5%, about 1% to about 6%, about 1% to about 7%, about 1% to about 8%, about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10% about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10%. In some embodiments, the fatty amide derived propylene glycol-diammonium phosphate ester is present in an amount of about 1%, about 2%, about 3%, about 4%, about 5%, about 6%, about 7%, about 8%, about 9%, or about 10%. In some embodiments, the fatty amide derived propylene glycol-diammonium phosphate ester is present in an amount of 7%. In some embodiments, the fatty amide derived propylene glycol-diammonium phosphate ester is present in an amount of at least about 1%, about 2%, about 3%, about 4%, about 5%, about 6%, about 7%, about 8% or about 9%. In some embodiments, the fatty amide derived propylene glycol-diammonium phosphate ester is present in an amount of at most about 2%, about 3%, about 4%, about 5% about 6%, about 7%, about 8% about 9%, or about 10%. In some embodiments, the fatty amide derived propylene glycol-diammonium phosphate ester

In some embodiments, the ratio of a non-ionic surface (e.g., propylene glycol monocaprylate) to cationic penetration enhancing agent (e.g., linoleamidopropyl PG-dimonium chloride phosphate) is about 1:1 to about 1:10 (w/w). In some embodiments, the ratio of a non-ionic surface to cationic penetration enhancing agent is about 1:1 to 1:2, 1:1 to 1:3, 1:1 to 1:4, 1:1 to 1:5, 1:1 to 1:6, 1:1 to 1:7, 1:1 to 1:8, 1:1 to 1:9, 1:1 to 1:10, 1:2 to 1:3, 1:2 to 1:4, 1:2 to 1:5, 1:2 to 1:6, 1:2 to 1:7, 1:2 to 1:8, 1:2 to 1:9, 1:2 to 1:10, 1:3 to 1:4, 1:3 to 1:5, 1:3 to 1:6, 1:3 to 1:7, 1:3 to 1:8, 1:3 to 1:9, 1:3 to 1:10, 1:4 to 1:5, 1:4 to 1:6, 1:4 to 1:7, 1:4 to 1:8, 1:4 to 1:9, 1:4 to 1:10, 1:5 to 1:6, 1:5 to 1:7, 1:5 to 1:8, 1:5 to 1:9, 1:5 to 1:10, 1:6 to 1:7, 1:6 to 1:8, 1:6 to 1:9, 1:6 to 1:10, 1:7 to 1:8, 1:7 to 1:9, 1:7 to 1:10, 1:8 to 1:9, 1:8 to 1:10, or 1:9 to 1:10 (w/w). In some embodiments, the ratio of a non-ionic surface to cationic penetration enhancing agent is about 1:1, 1:2, 1:3, 1:4, 1:5, 1:6, 1:7, 1:8, 1:9, or 1:10 (w/w). In some embodiments, the ratio of a non-ionic surface to cationic penetration enhancing agent is at least about 1:1, 1:2, 1:3, 1:4, 1:5, 1:6, 1:7, 1:8, 1:9, or 1:10 (w/w). In some embodiments, the ratio of a non-ionic surface to cationic penetration enhancing agent is at most about 1:1, 1:2, 1:3, 1:4, 1:5, 1:6, 1:7, 1:8, 1:9, or 1:10 (w/w).

In some embodiments, the cationic surfactant is a di-cationic penetration enhancing agent. In some embodiments, the di-cationic surfactant is a gemini surfactant. In some embodiments, a gemini surfactant is a surfactant comprising two quaternary amines represented by the formula A-N(R)₂—B—N(R)₂—C, wherein each of A and C is independently an optionally substituted C₆-C₂₄ alkyl group, each R is independently optionally substituted C₁-C₆ alkyl, and B is an optionally substituted C₂-C₁₀ alkylene chain. In some embodiments, the each of A and C is a C₆-C₂₄ saturated or unsaturated hydrocarbon. In some embodiments, the each of A and C is a C₆-C₂₄ saturated hydrocarbon. In some embodiments, each R is methyl. In some embodiments, B is a saturated C₂-C₁₀ alkylene chain. In some cases, gemini surfactants follow the nomenclature X—Y—Z, wherein each of X, Y, and Z is an integer representing the number of carbon atoms of each substituent, and Y is the spacer between the two quaternary amines. Thus, for example, a 12-3-12 gemini surfactant has the formula CH₃(CH₂)₁₁—[N(CH₃)₂]—(CH₂)₃—[N⁺(CH₃)₂]—(CH₂)₁₁CH₃. In some embodiments, the gemini surfactant is a 10-2-10, 12-2-12, 14-2-14, 10-3-10, 12-3-12, 14-3-14, 10-4-10, 12-4-12, or 14-4-14 gemini surfactant. In some embodiments, the gemini surfactant is a 12-3-12 gemini surfactant.

In some embodiments, the gemini surfactant is present in an amount of about 0.1% to about 1.5% (w/w) of the composition. In some embodiments, the gemini surfactant is present in an amount of about 0.1% to about 0.2%, about 0.1% to about 0.3%, about 0.1% to about 0.5%, about 0.1% to about 0.7%, about 0.1% to about 0.9%, about 0.1% to about 1%, about 0.1% to about 1.2%, about 0.1% to about 1.5%, about 0.2% to about 0.3%, about 0.2% to about 0.5%, about 0.2% to about 0.7%, about 0.2% to about 0.9%, about 0.2% to about 1%, about 0.2% to about 1.2%, about 0.2% to about 1.5%, about 0.3% to about 0.5%, about 0.3% to about 0.7%, about 0.3% to about 0.9%, about 0.3% to about 1%, about 0.3% to about 1.2%, about 0.3% to about 1.5%, about 0.5% to about 0.7%, about 0.5% to about 0.9%, about 0.5% to about 1%, about 0.5% to about 1.2%, about 0.5% to about 1.5%, about 0.7% to about 0.9%, about 0.7% to about 1%, about 0.7% to about 1.2%, about 0.7% to about 1.5%, about 0.9% to about 1%, about 0.9% to about 1.2%, about 0.9% to about 1.5%, about 1% to about 1.2%, about 1% to about 1.5%, or about 1.2% to about 1.5%. In some embodiments, the gemini surfactant is present in an amount of about 0.1%, about 0.2%, about 0.3%, about 0.5%, about 0.7%, about 0.9%, about 1%, about 1.2%, or about 1.5%. In some embodiments, the gemini surfactant is present in an amount of at least about 0.1%, about 0.2%, about 0.3%, about 0.5%, about 0.7%, about 0.9%, about 1%, or about 1.2%. In some embodiments, the gemini surfactant is present in an amount of at most about 0.2%, about 0.3%, about 0.5%, about 0.7%, about 0.9%, about 1%, about 1.2%, or about 1.5%.

In some embodiments, the cationic surfactant comprises a polycationic group. In some embodiments, the polycationic group is a polymer wherein each monomer of the polymer comprises a charged group (e.g., an amino group). In some embodiments, the polycationic group is polylysine. In some embodiments, the polycationic group is polyarginine.

In some embodiments, the polylysine has a molecular weight of from about 1 kDa to about 10 kDa, from about 1 kDa to about 5 kDa, or from about 3 kDa to about 5 kDa. In some embodiments, the polylysine is present in an amount of from about 0.01% to about 10%, from about 0.01% to about 0.5%, from about 0.01% to about 0.2%, from about 0.05% to about 1%, from about 0.05% to about 0.5%, or from about 0.05% to about 0.2% (w/w) of the composition.

### Additional Components

In some embodiments, the vesicle composition comprises additional components. In some embodiments, these additional components improve one or more properties of the vesicles without dramatically altering the delivery of the anionic polymer material.

In some embodiments, the vesicle composition further comprises one or more viscosity enhancing agents. In some embodiments, the viscosity enhancing agents thicken the composition for increased stability and/or feel to a user of the vesicle composition. In some embodiments, the viscosity enhancing agents also act as surfactants. In some embodiments, the viscosity enhancing agent comprises one or more of a fatty alcohol, a wax, a fatty ester of glycerol, or any combination thereof. In some embodiments, the fatty alcohol is a C₅-C₂₀ fatty alcohol. In some embodiments, the fatty alcohol is cetyl alcohol. In some embodiments, the cetyl alcohol is Crodacol C95. In some embodiments, the wax is a naturally occurring or synthetic wax. In some embodiments, the wax is beeswax. In some embodiment, the wax is synthetic beeswax. In some embodiments, the synthetic beeswax is Syncrowax™ BB4. In some embodiments, the synthetic beeswax is non-animal derived beeswax. In some embodiments the non-animal derived beeswax is Syncrowax™ SB1. In some embodiments, the fatty ester of glycerol is a monoester. In some embodiments, the monoester is an ester of a C₅-C₂₄ fatty acid. In some embodiments, the fatty ester of glycerol is glycerol monostearate.

In some embodiments, the viscosity enhancing agents are present in an amount of from about 0.5% to about 10% (w/w) of the composition. In some embodiments, the viscosity enhancing agents are present in an amount of from about 0.5% to about 5%, about 0.5% to about 5%, about 0.5% to about 4%, about 0.5% to about 3%, or from about 0.5% to about 2%. In some embodiments, the viscosity enhancing agents comprise a fatty alcohol in an amount of up to about 2%, a wax in an amount of up to about 2%, and a fatty ester of glycerol in an amount of up to about 5%. In some embodiments, the fatty alcohol is present in an amount of from about 0.1 to about 1.5%. In some embodiments, the wax is present in an amount of from about 0.1% to about 1%. In some embodiments, the fatty ester of glycerol is present in an amount of from about 0.5% to about 2%.

In some embodiments, the vesicle composition further comprises one or more of a thickener, a preservative, a moisturizer, an emollient, a humectant, or any combination thereof. In some embodiments, the vesicle composition further comprises a thickener. In some embodiments, the vesicle composition further comprises a preservative. In some embodiments, the vesicle composition further comprises a moisturizer. In some embodiments, the vesicle composition further comprises an emollient. In some embodiments, the vesicle composition further comprises a humectant.

In some embodiments, the vesicle composition further comprises an antimicrobial. In some embodiments, the antimicrobial is a paraben ester. In some embodiments, the antimicrobial is methylparaben or propylparaben, or a combination thereof. In some embodiments, the antimicrobial is present in an amount of up to about 1%, up to about 0.9%, up to about 0.8%, up to about 0.7%, up to about 0.6%, up to about 0.5%, up to about 0.4%, up to about 0.3%, up to about 0.2% (w/w) of the composition.

In some embodiments, the vesicle composition further comprises a thickener. In some embodiments, the thickener is an inert polymer material. In some embodiments, the thickener is a siloxane polymer. In some embodiments, the thickener polydimethyl siloxane (PDMS). In some embodiments, the PDMS is present in an amount of up to about 5%, up to about 4%, up to about 3%, up to about 2%, or up to about 1%. In some embodiments, the PDMS is present in an amount of from about 0.10% to about 2% (w/w) of the composition.

In some embodiments, the composition further comprises a humectant. In some embodiments, the composition comprises glycerol. In some embodiments, the glycerol is present in an amount of from about 0.5% to about 25%, about 0.5% to about 20%, about 0.5% to about 15%, or about 0.5% to about 10%. In some embodiments, the glycerol is present in an amount of about 1% to about 10%. In some embodiments, the glycerol is present in an amount of about 1% to about 2%, about 1% to about 4%, about 1% to about 6%, about 1% to about 8%, about 1% to about 10%, about 2% to about 4%, about 2% to about 6%, about 2% to about 8%, about 2% to about 10%, about 4% to about 6%, about 4% to about 8%, about 4% to about 10%, about 6% to about 8%, about 6% to about 10%, or about 8% to about 10%. In some embodiments, the glycerol is present in an amount of about 1%, about 2%, about 4%, about 6%, about 8%, or about 10%. In some embodiments, the glycerol is present in an amount of at least about 1%, about 2%, about 4%, about 6%, or about 8%. In some embodiments, the glycerol is present in an amount of at most about 2%, about 4%, about 6%, about 8%, or about 10% (w/w) of the composition.

In some embodiments, the vesicle composition comprises a preservative. In some embodiments, the preservative is a cosmetic preservative, such as Euxyl® PE 9010 or Spectrastat®. Euxyl® PE 9010 is a phenoxyethanol/ethylhexylglycerin mixture. Spectrastat® is a blend of caprylhydroxamic acid, caprylyl glycol, and glycerin. In some embodiments, the preservative is present in an amount of up to about 2%, up to about 1.5%, or up to about 1% (w/w) of the composition. In some embodiments, the preservative is present in an amount of from about 0.1% to about 2%, from about 0.1% to about 1.5%, or from about 0.1% to about 1%. In some embodiments, the preservative is present in an amount of about 0.1%, 0.2%, 0.3%, 0.4%, 0.5%, 0.6%, 0.7%, 0.8%, 0.9%, 1%, 1.1%, 1.2%, 1.3%, 1.4%, or 1.5%.

In some embodiments, the additional components comprise purified water. In some embodiments, purified water is present in an amount of about 50% to 90% (w/w). In some embodiments, purified water is present in an amount of about 50% to about 55%, about 50% to about 60%, about 50% to about 65%, about 50% to about 70%, about 50% to about 75%, about 50% to about 80%, about 50% to about 85%, about 50% to about 90%, about 55% to about 60%, about 55% to about 65%, about 55% to about 70%, about 55% to about 75%, about 55% to about 80%, about 55% to about 85%, about 55% to about 90%, about 60% to about 65%, about 60% to about 70%, about 60% to about 75%, about 60% to about 80%, about 60% to about 85%, about 60% to about 90%, about 65% to about 70%, about 65% to about 75%, about 65% to about 80%, about 65% to about 85%, about 65% to about 90%, about 70% to about 75%, about 70% to about 80%, about 70% to about 85%, about 70% to about 90%, about 75% to about 80%, about 75% to about 85%, about 75% to about 90%, about 80% to about 85%, about 80% to about 90%, or about 85% to about 90%. In some embodiments, purified water is present in an amount of about 50%, about 55%, about 60%, about 65%, about 70%, about 75%, about 80%, about 85%, or about 90%. In some embodiments, purified water is present in an amount of at least about 50%, about 55%, about 60%, about 65%, about 70%, about 75%, about 80%, or about 85%. In some embodiments, purified water is present in an amount of at most about 55%, about 60%, about 65%, about 70%, about 75%, about 80%, about 85%, or about 90%.

### Exemplary Compositions for Delivery of Anionic Polymer Materials

Provided below are exemplary compositions for the delivery of anionic polymer materials. The embodiments below may additional comprise any of the other ingredients or components provided herein.

Hyaluronic Acid Composition 1: In one aspect, provided herein, is a lipid vesicle composition comprising
(a) lipid vesicles each comprising a lipid bilayer comprising vesicle forming lipids, wherein the vesicle forming lipids are present in an amount of from about 5% to about 20%;
(b) an oil-in-water emulsion entrapped in the lipid vesicles, and stabilized by one or more surfactants, wherein the one or more surfactants comprises a cationic surfactant;
(c) hyaluronic acid in an amount of from about 0.1 mg/mL to about 10 mg/mL entrapped in the lipid bilayer and/or the oil-in-water emulsion.

In some embodiments, the oil component is present in an amount of from about 2.5% to about 20%.

In some embodiments, the lipid vesicle composition comprises hyaluronic acid in an amount of about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 1.25 mg/mL, about 0.01 mg/mL to about 1.5 mg/mL, about 0.01 mg/mL to about 1.75 mg/mL, about 0.01 mg/mL to about 2 mg/mL, about 0.01 mg/mL to about 5 mg/mL, about 0.01 mg/mL to about 10 mg/mL, about 0.1 mg/mL to about 0.5 mg/mL, about 0.1 mg/mL to about 1 mg/mL, about 0.1 mg/mL to about 1.25 mg/mL, about 0.1 mg/mL to about 1.5 mg/mL, about 0.1 mg/mL to about 1.75 mg/mL, about 0.1 mg/mL to about 2 mg/mL, about 0.1 mg/mL to about 5 mg/mL, about 0.1 mg/mL to about 10 mg/mL, about 0.5 mg/mL to about 1 mg/mL, about 1 mg/mL to about 1.5 mg/mL, about 1 mg/mL to about 1.75 mg/mL, about 1 mg/mL to about 2 mg/mL, about 1 mg/mL to about 5 mg/mL, about 1 mg/mL to about 10 mg/mL. In some embodiments, the lipid vesicle composition comprises hyaluronic acid in an amount of about 0.01 mg/mL, about 0.05 mg/mL, about 0.1 mg/mL, about 0.5 mg/mL, about 1 mg/mL, about 1.25 mg/mL, about 1.5 mg/mL, about 1.75 mg/mL, about 2 mg/mL, about 5 mg/mL, or about 10 mg/mL.

In some embodiments, the lipid vesicle composition further comprises viscosity enhancing agents in an amount of from about 0.5% to about 5%. In some embodiments, the viscosity enhancing agents comprise one or more of a fatty alcohol, a wax, a fatty ester of glycerol, or any combination thereof.

In some embodiments, the lipid vesicle composition further comprises a non-ionic surfactant in an amount of from about 0.1% to about 3%. In some embodiments, the non-ionic surfactant is a PEG ether of a fatty alcohol.

In some embodiments, the cationic surfactant is a fatty amide derived propylene glycol-diammonium phosphate ester. In some embodiments, the cationic surfactant is present in an amount of from about 1% to about 10%.

In some embodiments, the lipid vesicle composition further comprises a peptide antagonist of muscle-type nicotinic acetylcholine receptors in an amount of from about 0.1 mg/mL to about 50 mg/mL entrapped in the lipid bilayer and/or the oil-in-water emulsion. In some embodiments, the lipid vesicle composition comprises the peptide antagonist in an amount of about 0.1 mg/mL to about 0.5 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL. In some embodiments the lipid vesicle composition comprises the peptides antagonist in an amount of about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the lipid vesicle composition comprises the peptides antagonist in an amount of about 1 mg/mL, about 2 mg/mL, about 3 mg/mL, about 4 mg/mL, or about 5 mg/mL.

Hyaluronic Acid Composition 2: In one aspect, provided herein, is a lipid vesicle composition comprising
(a) lipid vesicles each comprising a lipid bilayer comprising vesicle forming lipids, wherein the vesicle forming lipids are present in an amount of from about 2% to about 20%;
(b) an oil-in-water emulsion entrapped in the lipid vesicles, and stabilized by one or more surfactants;
(c) hyaluronic acid in an amount of from about 0.1 mg/mL to about 10 mg/mL entrapped in the lipid bilayer and/or the oil-in-water emulsion,
wherein the composition further comprises:
   a gemini surfactant in an amount of from about 0.01% to about 0.5%; and
   a polysorbate in an amount of from about 0.1% to about 2%.

In some embodiments, the oil component is present in an amount of from about 2.5% to about 20%.

In some embodiments, the lipid vesicle composition comprises hyaluronic acid in an amount of about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 1.25 mg/mL, about 0.01 mg/mL to about 1.5 mg/mL, about 0.01 mg/mL to about 1.75 mg/mL, about 0.01 mg/mL to about 2 mg/mL, about 0.01 mg/mL to about 5 mg/mL, about 0.01 mg/mL to about 10 mg/mL, about 0.1 mg/mL to about 0.5 mg/mL, about 0.1 mg/mL to about 1 mg/mL, about 0.1 mg/mL to about 1.25 mg/mL, about 0.1 mg/mL to about 1.5 mg/mL, about 0.1 mg/mL to about 1.75 mg/mL, about 0.1 mg/mL to about 2 mg/mL, about 0.1 mg/mL to about 5 mg/mL, about 0.1 mg/mL to about 10 mg/mL, about 0.5 mg/mL to about 1 mg/mL, about 1 mg/mL to about 1.5 mg/mL, about 1 mg/mL to about 1.75 mg/mL, about 1 mg/mL to about 2 mg/mL, about 1 mg/mL to about 5 mg/mL, about 1 mg/mL to about 10 mg/mL. In some embodiments, the lipid vesicle composition comprises hyaluronic acid in an amount of about 0.01 mg/mL, about 0.05 mg/mL, about 0.1 mg/mL, about 0.5 mg/mL, about 1 mg/mL, about 1.25 mg/mL, about 1.5 mg/mL, about 1.75 mg/mL, about 2 mg/mL, about 5 mg/mL, or about 10 mg/mL.

In some embodiments, the lipid vesicle composition further comprises viscosity enhancing agents in an amount of from about 0.5% to about 5%. In some embodiments, the viscosity enhancing agents comprise one or more of a fatty alcohol, a wax, a fatty ester of glycerol, or any combination thereof.

In some embodiments, the polysorbate is polysorbate 80.

In some embodiments, the lipid vesicle composition further comprises a peptide antagonist of muscle-type nicotinic acetylcholine receptors in an amount of from about 0.1 mg/mL to about 50 mg/mL entrapped in the lipid bilayer and/or the oil-in-water emulsion. In some embodiments, the lipid vesicle composition comprises the peptide antagonist in an amount of about 0.1 mg/mL to about 0.5 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL. In some embodiments the lipid vesicle composition comprises the peptides antagonist in an amount of about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the lipid vesicle composition comprises the peptides antagonist in an amount of about 1 mg/mL, about 2 mg/mL, about 3 mg/mL, about 4 mg/mL, or about 5 mg/mL.

### Lipid Vesicle Compositions of Muscle-Type Nicotinic Acetylcholine Receptor Antagonist Peptides for Intradermal Delivery

In one aspect, provided herein, is a lipid vesicle composition comprising a peptide antagonist of muscle-type nicotinic acetylcholine receptors. In some embodiments, the lipid vesicle composition comprises lipid vesicles each comprising a lipid bilayer comprising vesicle forming lipids. In some embodiments, the lipid vesicle composition comprises an oil-in-water emulsion entrapped in the lipid vesicles. In some embodiments, the oil-in-water emulsion is stabilized by one or more surfactants. In some embodiments, the peptide antagonist is entrapped in the lipid bilayer and/or the oil-in-water emulsion. In some embodiments, the peptide antagonist is entrapped in the lipid bilayer. In some embodiments, the peptide antagonist is entrapped in the oil-in-water emulsion.

### Muscle-Type Nicotinic Acetylcholine Receptor

In one aspect, the present disclosure relates to lipid vesicle compositions comprising peptide antagonists of muscle-type nicotinic acetylcholine receptors, also referred to as muscle nAChR.

The muscle nAChR is a ligand-activated ion channel receptor having a structure generally described as a heteropentamer of four related, but genetically and immunologically distinct, subunits. The subunits are organized around a central pore in the membrane with a stoichiometry of two a subunits and one each of β, δ, and γ. Muscle nAChR is activated by the endogenous neurotransmitter acetylcholine (ACh, the natural receptor agonist) released by the nerve at the neuromuscular junction. ACh binds to the receptor resulting in transmission of a signal for channel activation, or gating.

The peptide antagonists of the disclosure bind in the active site of the muscle nAChR, inhibiting binding of ACh to the receptor. This results in a non-depolarizing blockage of the neuromuscular postsynaptic membrane, such that the signal from the nerve (the ACh release) is no longer effective in stimulating muscle contraction. See, e.g., Albuquerque, et al., 2009, “Mammalian Nicotinic Acetylcholine Receptors: From Structure to Function,” Physiol. Rev. 89(1):73-120, and Kalamida, et al., 2007, “Muscle and neuronal nicotinic acetylcholine receptors,” The FEBS Journal 274:3799-3845, each incorporated herein by reference in its entirety.

Intentional muscle deinnervation has been achieved using the anticholinergic botulinum toxin products: onabotulinumtoxin A (BTX-A, marketed as BOTOX®), abobotulinumtoxin A (Dysport®), incobotulinumtoxin A (Xeomin®), rimabotulinumtoxinB (Myobloc®) and prabotulinumtoxinA-xvfs (Jeuveau®). BTX-A prevents the secretion of ACh, present in nerve cell vesicles, from the nerve cell at the synapse. This results in an absence of ACh at the synapse and failure to innervate the muscle cell. The mechanism of action of these toxins are thus pre-synaptic. Botulinum toxin is indicated for use in, e.g, preventing or improving of the appearance of skin wrinkles, e.g., in the face, skin laxity, moderate to severe glabellar lines associated with corrugator and/or procerus muscle activity, moderate to severe lateral canthal lines associated with orbicularis oculi activity (crow's feet lines), moderate to severe forehead lines associated with frontalis muscle activity; treatment of overactive bladder (OAB); treatment of urinary incontinence; prophylaxis of headaches in adult patients with chronic migraine; prevention or treatment of episodic migraine; treatment of upper and lower limb spasticity; treatment of cervical dystonia; treatment of hypersalivation (also called ptyalism or sialorrhea); treatment of blepharospasm associated with dystonia; treatment of and treatment of strabismus. (See, e.g., Botox Cosmetic BLA 103000, product labeling for Botox Cosmetic revised 5/2018, product labeling for Botox revised 4/2017, Dysport BLA 125274, product labeling for Dysport revised 6/2017, Xeomin BLA 125360, product labeling for Xeomin revised 7/2018, product labeling for Myobloc revised 8/2019, and product labeling for Jeuveau BLA 761085 revised 7/2019, each incorporated herein by reference.)

In contrast, peptide antagonists of the disclosure occupy the ACh active site in muscle cell AChRs (post-synapse). When bound, a peptide antagonist of the disclosure blocks the binding of ACh that has been secreted from the nerve cell.

### Peptide Antagonists of the Muscle-Type Nicotinic Acetylcholine Receptor

The present disclosure provides lipid vesicle compositions comprising peptide antagonists of mammalian muscle nAChR, including human muscle nAChR. In some embodiments, a peptide antagonist of provided herein has a desirable property, or an improved property relative to a muscle nAChR antagonist known in the art. Such a property can include, e.g., a pharmacokinetic property (including but not limited to absorption, bioavailability, distribution, metabolism, and excretion), a pharmacodynamic property (including but not limited to: receptor binding characteristics, e.g., binding half-life; postreceptor effects; and chemical interactions), enhanced activity (e.g., represented by IC₅₀), stability (e.g., represented by half-life), solubility (e.g., in a formulation), or permeability (e.g., permeability of the skin by a formulation containing the peptide antagonist). In some embodiments, a formulation containing a peptide antagonist of the disclosure has a desirable property, or an improved property relative to a formulation containing a muscle nAChR antagonist known in the art. In some embodiments, a desirable or improved property of a formulation of the disclosure is a property relating to the use of the formulation for an indication as described elsewhere herein, e.g., use for reducing or improving the appearance of skin wrinkles.

### Peptide Antagonists

In some embodiments, the peptide antagonist of the lipid vesicle composition comprises a conotoxin peptide. In some embodiments, the peptide antagonist comprises an amino acid sequence which has at least about 50%, at least about 60%, at least about 70%, at least about 80%, or at least about 90% sequence homology to the amino acid sequence of any one of SEQ ID NOs: 1-52 or 60-99. In some embodiments, the peptide antagonist comprises one or more amino acid substitutions relative to any one SEQ ID NO: 1-52 or 60-99. In some embodiments, the peptide antagonist comprises 1, 2, 3, 4, or 5 amino acid substitutions relative any one of SEQ ID NO: 1-52 or 60-99. In some embodiments, at least 1, 2, or 3 of the 1, 2, 3, 4, or 5 amino acid substitutions is a conservative substitution. In some embodiments, the peptide antagonist comprises an amino acid sequence identical to the amino acid sequence of any one of SEQ ID NOs: 1-52 or 60-99. In some embodiments, the peptide antagonist has an amino acid sequence consisting of an identical sequence of any one of SEQ ID NOs: 1-52 or 60-99.

In some embodiments, the peptide antagonist comprises an amino acid sequence which has at least about 50%, at least about 60%, at least about 70%, at least about 80%, or at least about 90% sequence homology to the amino acid sequence SEQ ID NO: 1. In some embodiments, the peptide antagonist comprises one or more amino acid substitutions relative to SEQ ID NO: 1. In some embodiments, the one or more amino acid substitutions are selected from the amino acids defined as Xaa1-Xaa14. In some embodiments, the peptide antagonist comprises 1, 2, 3, 4, or 5 amino acid substitutions relative to SEQ ID NO: 1. In some embodiments, at least 1, 2, or 3 of the 1, 2, 3, 4, or 5 amino acid substitutions is a conservative substitution. In some embodiments, the peptide antagonist has an amino acid sequence consisting of an identical sequence of SEQ ID NO: 1.

In some embodiments, the peptide antagonist comprises an amino acid sequence which has at least about 50%, at least about 60%, at least about 70%, at least about 80%, or at least about 90% sequence homology to the amino acid sequence of SEQ ID NO: 3. In some embodiments, the peptide antagonist comprises one or more amino acid substitutions relative to SEQ ID NO: 3. In some embodiments, the one or more amino acid substitutions are selected from the amino acids defined as Xaa1-Xaa14. In some embodiments, the peptide antagonist comprises 1, 2, 3, 4, or 5 amino acid substitutions relative to SEQ ID NO: 3. In some embodiments, at least 1, 2, or 3 of the 1, 2, 3, 4, or 5 amino acid substitutions is a conservative substitution. In some embodiments, the peptide antagonist has an amino acid sequence consisting of an identical sequence of SEQ ID NO: 3.

In some embodiments, the peptide antagonist comprises an amino acid sequence which has at least about 50%, at least about 60%, at least about 70%, at least about 80%, or at least about 90% sequence homology to the amino acid sequence of SEQ ID NO: 60. In some embodiments, the peptide antagonist comprises one or more amino acid substitutions relative to SEQ ID NO: 60. In some embodiments, the one or more amino acid substitutions are selected from the amino acids defined as Xaa1-Xaa14. In some embodiments, the peptide antagonist comprises 1, 2, 3, 4, or 5 amino acid substitutions relative to SEQ ID NO: 60. In some embodiments, at least 1, 2, or 3 of the 1, 2, 3, 4, or 5 amino acid substitutions is a conservative substitution. In some embodiments, the peptide antagonist has an amino acid sequence consisting of an identical sequence of SEQ ID NO: 60.

In some embodiments, the peptide antagonist comprises an amino acid sequence which has at least about 50%, at least about 60%, at least about 70%, at least about 80%, or at least about 90% sequence homology to the amino acid sequence of SEQ ID NO: 61. In some embodiments, the peptide antagonist comprises one or more amino acid substitutions relative to SEQ ID NO: 61. In some embodiments, the one or more amino acid substitutions are selected from the amino acids defined as Xaa1-Xaa14. In some embodiments, the peptide antagonist comprises 1, 2, 3, 4, or 5 amino acid substitutions relative to SEQ ID NO: 61. In some embodiments, at least 1, 2, or 3 of the 1, 2, 3, 4, or 5 amino acid substitutions is a conservative substitution. In some embodiments, the peptide antagonist has an amino acid sequence consisting of an identical sequence of SEQ ID NO: 61.

In some embodiments, the peptide antagonist comprises an amino acid sequence which has at least about 50%, at least about 60%, at least about 70%, at least about 80%, or at least about 90% sequence homology to the amino acid sequence of SEQ ID NO: 73. In some embodiments, the peptide antagonist comprises one or more amino acid substitutions relative to SEQ ID NO: 73. In some embodiments, the one or more amino acid substitutions are selected from the amino acids defined as Xaa1-Xaa14. In some embodiments, the peptide antagonist comprises 1, 2, 3, 4, or 5 amino acid substitutions relative to SEQ ID NO: 73. In some embodiments, at least 1, 2, or 3 of the 1, 2, 3, 4, or 5 amino acid substitutions is a conservative substitution. In some embodiments, the peptide antagonist has an amino acid sequence consisting of an identical sequence of SEQ ID NO: 73.

In some embodiments, the peptide antagonist comprises an amino acid sequence which has at least about 50%, at least about 60%, at least about 70%, at least about 80%, or at least about 90% sequence homology to the amino acid sequence of SEQ ID NO: 78. In some embodiments, the peptide antagonist comprises one or more amino acid substitutions relative to SEQ ID NO: 78. In some embodiments, the one or more amino acid substitutions are selected from the amino acids defined as Xaa1-Xaa14. In some embodiments, the peptide antagonist comprises 1, 2, 3, 4, or 5 amino acid substitutions relative to SEQ ID NO: 78. In some embodiments, at least 1, 2, or 3 of the 1, 2, 3, 4, or 5 amino acid substitutions is a conservative substitution. In some embodiments, the peptide antagonist has an amino acid sequence consisting of an identical sequence of SEQ ID NO: 78.

In some embodiments, the peptide antagonist comprises an amino acid sequence which has at least about 50%, at least about 60%, at least about 70%, at least about 80%, or at least about 90% sequence homology to the amino acid sequence of SEQ ID NO: 82. In some embodiments, the peptide antagonist comprises one or more amino acid substitutions relative to SEQ ID NO: 82. In some embodiments, the one or more amino acid substitutions are selected from the amino acids defined as Xaa1-Xaa14. In some embodiments, the peptide antagonist comprises 1, 2, 3, 4, or 5 amino acid substitutions relative to SEQ ID NO: 82. In some embodiments, at least 1, 2, or 3 of the 1, 2, 3, 4, or 5 amino acid substitutions is a conservative substitution. In some embodiments, the peptide antagonist has an amino acid sequence consisting of an identical sequence of SEQ ID NO: 82.

In some embodiments, the peptide antagonist comprises an amino acid sequence which has at least about 50%, at least about 60%, at least about 70%, at least about 80%, or at least about 90% sequence homology to the amino acid sequence of SEQ ID NO: 85. In some embodiments, the peptide antagonist comprises one or more amino acid substitutions relative to SEQ ID NO: 85. In some embodiments, the one or more amino acid substitutions are selected from the amino acids defined as Xaa1-Xaa14. In some embodiments, the peptide antagonist comprises 1, 2, 3, 4, or 5 amino acid substitutions relative to SEQ ID NO: 85. In some embodiments, at least 1, 2, or 3 of the 1, 2, 3, 4, or 5 amino acid substitutions is a conservative substitution. In some embodiments, the peptide antagonist has an amino acid sequence consisting of an identical sequence of SEQ ID NO: 85.

In some embodiments, the peptide antagonist comprises an amino acid sequence which has at least about 50%, at least about 60%, at least about 70%, at least about 80%, or at least about 90% sequence homology to the amino acid sequence of SEQ ID NO: 91. In some embodiments, the peptide antagonist comprises one or more amino acid substitutions relative to SEQ ID NO: 91. In some embodiments, the one or more amino acid substitutions are selected from the amino acids defined as Xaa1-Xaa14. In some embodiments, the peptide antagonist comprises 1, 2, 3, 4, or 5 amino acid substitutions relative to SEQ ID NO: 91. In some embodiments, at least 1, 2, or 3 of the 1, 2, 3, 4, or 5 amino acid substitutions is a conservative substitution. In some embodiments, the peptide antagonist has an amino acid sequence consisting of an identical sequence of SEQ ID NO: 91.

In some embodiments, the peptide antagonist comprises an amino acid sequence which has at least about 50%, at least about 60%, at least about 70%, at least about 80%, or at least about 90% sequence homology to the amino acid sequence of SEQ ID NO: 95. In some embodiments, the peptide antagonist comprises one or more amino acid substitutions relative to SEQ ID NO: 95. In some embodiments, the one or more amino acid substitutions are selected from the amino acids defined as Xaa1-Xaa14. In some embodiments, the peptide antagonist comprises 1, 2, 3, 4, or 5 amino acid substitutions relative to SEQ ID NO: 95. In some embodiments, at least 1, 2, or 3 of the 1, 2, 3, 4, or 5 amino acid substitutions is a conservative substitution. In some embodiments, the peptide antagonist has an amino acid sequence consisting of an identical sequence of SEQ ID NO: 95.

In some embodiments, the peptide antagonist comprises up to about 20 amino acids, up to about 18 amino acids, up to about 16 amino acids, or up to about 14 amino acids. In some embodiments, the peptide antagonist has a molecular weight of up to about 2500 Da, up to about 2200 Da, up to about 2000 Da, up to about 1800 Da, up to about 1700 Da, up to about to about 1600 Da, or up to about 1500 Da.

In some embodiments, a muscle-type nicotinic acetylcholine receptor peptide antagonist of the lipid vesicle composition has 12-14 residues and comprises the amino acid sequence:

Xaa1-Xaa2-Xaa3-Xaa4-Xaa5-Xaa6-Xaa7-Xaa8-Xaa9-Xaa10-Xaa11-Xaa12-Xaa13-Xaa14
wherein:
Xaa1 is absent or selected from Ala, Gly, Val, Leu, Ile and a derivative of Ala, Gly, Val, Leu, or Ile;
Xaa2 is absent or selected from: Asn, Asp, Gln, Glu, Arg, His, Lys, Phe, Trp, Tyr, Ala, Gly, Val, Leu, Ile, and a derivative of Asn, Asp, Gln, Glu, Arg, His, Lys, Phe, Trp, Tyr, Ala, Gly, Val, Leu, or Ile;
Xaa3 and Xaa8 form a linkage Xaa3-Xaa8;
Xaa4 and Xaa14 form a linkage Xaa4-Xaa14;
Xaa5 is selected from: Asn, Asp, Gln, Glu, Arg, His, Lys, and a derivative of Asn, Asp, Gln, Glu, Arg, His, or Lys;
Xaa6 is selected from: Pro and a derivative thereof;
Xaa7 is selected from: Ala, Gly, Val, Leu, Ile and a derivative of Ala, Gly, Val, Leu, or Ile;
Xaa9 is selected from: Ala, Gly, Val, Leu, Ile and a derivative of Ala, Gly, Val, Leu, or Ile;
Xaa10 is selected from: Arg, His, Lys, and a derivative of Arg, His, or Lys;
Xaa11 is selected from: Asn, Asp, Gln, Glu, Arg, His, Lys, and a derivative of Asn, Asp, Gln, Glu, Arg, His, or Lys;
Xaa12 is selected from: Phe, Trp, Tyr, and a derivative of Phe, Trp, or Tyr;
Xaa13 is selected from: Cys, Met, Sec, Ser, Thr, Arg, His, Lys, and a derivative of Cys, Met, Sec, Ser, Thr, Arg, His, or Lys;
the N-terminus is optionally modified; and
the C-terminus is optionally modified.

In some embodiments, a muscle-type nicotinic acetylcholine receptor peptide antagonist of the lipid vesicle composition has 12-14 residues and comprises an amino acid sequence:

Xaa1-Xaa2-Xaa3-Xaa4-Xaa5-Xaa6-Xaa7-Xaa8-Xaa9-Xaa10-Xaa11-Xaa12-Xaa13-Xaa14
wherein:
Xaa1 is absent;
Xaa2 is absent;
Xaa3 and Xaa8 form a linkage Xaa3-Xaa8;
Xaa4 and Xaa14 form a linkage Xaa4-Xaa14;
Xaa5 is selected from: Asp, Gln, Glu, Arg, His, and Lys;
Xaa6 is selected from: Pro and hydroxyproline;
Xaa7 is selected from: Ala, Gly, Val, Leu, and Ile;
Xaa9 is selected from: Ala, Gly, Val, Leu, and Ile;
Xaa10 is selected from: Arg and His;
Xaa11 is selected from: Asn, Asp, Gln, Glu, Arg, His, and Lys;
Xaa12 is selected from: Trp and Tyr;
Xaa13 is selected from: Cys, Met, Sec, Ser, Thr, Arg, His, and Lys;
the N-terminus is optionally modified; and
the C-terminus is optionally modified.

In some embodiments, the peptide antagonist of the disclosure does not consist of the following amino acid sequence:

(SEQ ID NO: 1)
Glu-Cys-Cys-Asn-Pro-Ala-Cys-Gly-Arg-His-Tyr-Ser-
Cys

wherein the first and third cysteine residues (Xaa3-Xaa8) are linked and the second and fourth cysteine residues (Xaa4-Xaa14) are linked (α-conotoxin GI, or CGI).

In some embodiments, the peptide antagonist of the disclosure does not consist of the following amino acid sequence:

(SEQ ID NO: 2)
Glu-Cys-Cys-Asn-Pro-Ala-Cys-Gly-Lys-His-Phe-Ser-
Cys

wherein the first and third cysteine residues (Xaa3-Xaa8) are linked and the second and fourth cysteine residues (Xaa4-Xaa14) are linked.

In some embodiments, the peptide antagonist of the disclosure does not consist of the following amino acid sequence:

(SEQ ID NO: 56)
Glu-Cys-Cys-His-Pro-Ala-Cys-Gly-Lys-His-Phe-Ser-
Cys

wherein the first and third cysteine residues (Xaa3-Xaa8) are linked and the second and fourth cysteine residues (Xaa4-Xaa14) are linked (an α-conotoxin GII sequence).

In some embodiments, the peptide antagonist of the disclosure does not consist of the following amino acid sequence:

(SEQ ID NO: 3)
Gly-Arg-Cys-Cys-His-Pro-Ala-Cys-Gly-Lys-Asn-Tyr-
Ser-Cys

wherein the first and third cysteine residues (Xaa3-Xaa8) are linked and the second and fourth cysteine residues (Xaa4-Xaa14) are linked (α-conotoxin MI, or CMI).

In some embodiments, the number of amino acid residues in a peptide antagonist of the disclosure is not more than 12, not more than 13 or not more than 14. In embodiments, a peptide antagonist of the disclosure consists of 12, 13 or 14 amino acid residues.

Non-limiting examples of peptide antagonists of the disclosure are shown in Table 2.

TABLE 2
Peptide Antagonist Examples

| SEQ | |
|---|---|
| ID | |
| NO | SEQUENCE |
| 1^{3,4} | Glu-Cys-Cys-Asn-Pro-Ala-Cys-Gly-Arg-His-Tyr-Ser-Cys |
| 2^{3,4} | Glu-Cys-Cys-Asn-Pro-Ala-Cys-Gly-Lys-His-Phe-Ser-Cys |
| 3^{3,4} | Gly-Arg-Cys-Cys-His-Pro-Ala-Cys-Gly-Lys-Asn-Tyr-Ser-Cys |
| 4^{3,4} | Ac-Cys-Cys-Lys-Pro-Ala-Cys-Gly-Lys-Asn-Tyr-Ser-Cys-NH₂ |
| 5^{2,3} | Ac-Cys-(Cyt)-Arg-Pro-Ala-Cys-Gly-His-Asn-Tyr-Ser-(Cyt)-NH₂ |
| 6^{2,3} | Ac-Cys-(Cyt)-His-Pro-Ala-Cys-Gly-His-Asn-Tyr-Ser-(Cyt)-NH₂ |
| 7^{1,2} | Ac-(Cyt)-(Cyt)-Lys-Pro-Ala-(Cyt)-Gly-Lys-Gln-Tyr-Ser-(Cyt)-NH₂ |
| 8^{3,4} | Ac-Cys-Cys-Arg-Pro-Ala-Cys-Gly-Lys-Gln-Tyr-Ser-Cys-NH₂ |
| 9^{1,3} | Ac-(Cyt)-Cys-His-Pro-Ala-(Cyt)-Gly-Lys-Gln-Tyr-Ser-Cys-NH₂ |
| 10^{1,2} | Ac-(Cyt)-(Cyt)-His-Pro-Ala-(Cyt)-Gly-Arg-Gln-Tyr-Ser-(Cyt)-NH₂ |
| 11^{2,3} | H-Cys-(Cyt)-His-Pro-Ala-Cys-Gly-Arg-Asn-Tyr-Ser-(Cyt)-NH₂ |
| 12^{3,4} | Ac-Cys-Cys-Arg-Pro-Ala-Cys-Gly-Arg-Asn-Tyr-Ser-Cys-NH₂ |
| 13^{1,2} | Ac-(Cyt)-(Cyt)-Lys-Pro-Ala-(Cyt)-Gly-Arg-Asn-Tyr-Ser-(Cyt)-NH₂ |
| 14^{2,5} | Ac-Sec-(Cyt)-Lys-Pro-Ala-Sec-Gly-Lys-Asn-Tyr-Ser-(Cyt)-NH₂ |
| 15^{1,2} | Ac-(Cyt)-(Cyt)-His-Pro-Ala-(Cyt)-Gly-Lys-Asn-Tyr-Ser-(Cyt)-NH₂ |
| 16^{1,2} | H-(Cyt)-(Cyt)-His-Pro-Ala-(Cyt)-Gly-Lys-Asn-Tyr-Ser-(Cyt)-NH₂ |
| 17^{5,6} | Ac-Sec-Sec-Asn-Pro-Ala-Sec-Gly-Arg-His-Tyr-Ser-Sec-NH₂ |
| 18^{1,3} | NH²-(Cyt)-Cys-Asn-Pro-Ala-(Cyt)-Gly-Arg-His-Tyr-Ser-Cys-NH₂ |
| 19^{3,4} | Ac-Cys-Cys-Gln-Pro-Ala-Cys-Gly-Lys-His-Tyr-Ser-Cys-NH₂ |
| 20^{2,3} | Ac-Cys-(Cyt)-Asn-Pro-Ala-(Cyt)-Gly-Lys-His-Tyr-Ser-(Cyt)-NH₂ |
| 21^{1,2} | Ac-(Cyt)-(Cyt)-Asn-Pro-Ala-(Cyt)-Gly-Arg-His-Tyr-Ser-(Cyt)-NH₂ |
| 22^{1,4} | Ac-(Cyt)-Cys-Asn-Pro-Ala-(Cyt)-Gly-Arg-His-Tyr-Ser-Cys-NH₂ |
| 23^{2,3} | Ac-Cys-(Cyt)-Asn-Pro-Ala-Cys-Gly-Lys-His-Tyr-Ser-(Cyt)-NH₂ |
| 24^{1,2} | Ac-(Cyt)-(Cyt)-Asn-Pro-Ala-(Cyt)-Gly-Lys-His-Tyr-Ser-(Cyt)-NH₂ |
| 25^{1,2} | NH²-(Cyt)-(Cyt)-Asn-Pro-Ala-(Cyt)-Gly-Arg-His-Tyr-Ser-(Cyt)-NH₂ |
| 26^{1,2} | H-(Cyt)-(Cyt)-Asn-Pro-Ala-(Cyt)-Gly-Arg-His-Tyr-Ser-(Cyt)-NH₂ |
| 27^{1,2} | Ac-Arg-(Cyt)-(Cyt)-His-Pro-Ala-(Cyt)-Gly-Lys-Asn-Tyr-Ser-(Cyt)-NH₂ |
| 28^{1,2} | H-Arg-(Cyt)-(Cyt)-His-Pro-Ala-(Cyt)-Gly-Lys-Asn-Tyr-Ser-(Cyt)-NH₂ |
| 29^{3,4} | Ac-Arg-Cys-Cys-His-Pro-Ala-Cys-Gly-Lys-Asn-Tyr-Ser-Cys-NH₂ |
| 30^{2,3} | Ac-Arg-Cys-(Cyt)-Lys-Pro-Ala-Cys-Gly-Lys-Asn-Tyr-Ser-(Cyt)-NH₂ |
| 31^{2,3} | Ac-Arg-Cys-(Cyt)-His-Pro-Ala-Cys-Gly-Lys-Asn-Tyr-Ser-(Cyt)-NH₂ |
| 32^{1,3} | Ac-Arg-(Cyt)-Cys-His-Pro-Ala-(Cyt)-Gly-Arg-Asn-Tyr-Ser-Cys-NH₂ |
| 33^{1,2} | Ac-Arg-(Cyt)-(Cyt)-Arg-Pro-Ala-(Cyt)-Gly-Lys-Asn-Tyr-Ser-(Cyt)-NH₂ |
| 34^{1,2} | Ac-Arg-(Cyt)-(Cyt)-His-Pro-Ala-(Cyt)-Gly-His-Asn-Tyr-Ser-(Cyt)-NH₂ |
| 35^{1,2} | H-Arg-(Cyt)-(Cyt)-Arg-Pro-Ala-(Cyt)-Gly-Lys-Asn-Tyr-Ser-(Cyt)-NH₂ |
| 36^{5,6} | Ac-Arg-Sec-Sec-His-Pro-Ala-Sec-Gly-Lys-Asn-Tyr-Ser-Sec-NH₂ |
| 37^{1,2} | Ac-Lys-(Cyt)-(Cyt)-His-Pro-Ala-(Cyt)-Gly-Lys-Asn-Tyr-Ser-(Cyt)-NH₂ |
| 38^{1,2} | Ac-Lys-(Cyt)-(Cyt)-His-Pro-Ala-(Cyt)-Gly-Arg-Asn-Tyr-Ser-(Cyt)-NH₂ |
| 39^{1,2} | H-(Cyt)-(Cyt)-His-Pro-Ala-(Cyt)-Gly-Arg-His-Tyr-Ser-(Cyt)-NH₂ |
| 40^{3,4} | Ac-Cys-Cys-Lys-Pro-Ala-Cys-Gly-Arg-His-Tyr-Ser-Cys-NH₂ |
| 41^{2,3} | Ac-Cys-(Cyt)-His-Pro-Ala-Cys-Gly-Arg-His-Tyr-Ser-(Cyt)-NH₂ |
| 42^{2,3} | H-Cys-(Cyt)-His-Pro-Ala-Cys-Gly-Arg-His-Tyr-Ser-(Cyt)-NH₂ |
| 43^{1,2} | Ac-(Cyt)-(Cyt)-His-Pro-Ala-(Cyt)-Gly-Lys-His-Tyr-Ser-(Cyt)-NH₂ |
| 44^{1,2} | Ac-(Cyt)-(Cyt)-His-Pro-Ala-(Cyt)-Gly-Arg-His-Tyr-Ser-(Cyt)-NH₂ |
| 45^{1,2} | H-(Cyt)-(Cyt)-His-Pro-Ala-(Cyt)-Gly-Lys-His-Tyr-Ser-(Cyt)-NH₂ |
| 46^{1,3} | Ac-(Cyt)-Cys-His-Pro-Ala-(Cyt)-Gly-Arg-Lys-Tyr-Ser-Cys-NH₂ |
| 47^{1,3} | H-(Cyt)-Cys-His-Pro-Ala-(Cyt)-Gly-Arg-Lys-Tyr-Ser-Cys-NH₂ |
| 48^{1,3} | Ac-(Cyt)-Cys-His-Pro-Ala-(Cyt)-Gly-Arg-Lys-Tyr-Ser-Cys-NH₂ |
| 49^{1,2} | NH²-(Cyt)-(Cyt)-His-Pro-Ala-(Cyt)-Gly-Arg-His-Tyr-Ser-(Cyt)-NH₂ |
| 50^{1,2} | Ac-(Cyt)-(Cyt)-Lys-Pro-Ala-(Cyt)-Gly-Arg-His-Tyr-Ser-(Cyt)-NH₂ |
| 51^{2,5} | Ac-Sec-(Cyt)-His-Pro-Ala-Sec-Gly-Lys-His-Tyr-Ser-(Cyt)-NH₂ |
| 52^{1,6} | Ac-(Cyt)-Sec-His-Pro-Ala-(Cyt)-Gly-Arg-His-Tyr-Ser-Sec-NH₂ |
| 60^{3,4} | Ac-Cys-Cys-Asn-Pro-Ala-Cys-Gly-Lys-Asn-Tyr-Ser-Cys-NH₂ |
| 61^{3,4} | Ac-Cys-Cys-His-Pro-Ala-Cys-Gly-Arg-His-Tyr-Ser-Cys-NH₂ |
| 62^{3,4} | Ac-Cys-Cys-Asn-Pro-Ala-Cys-Gly-Lys-Asn-Tyr-Lys-Cys-NH₂ |
| 63^{1,2} | Ac-(Cyt)-(Cyt)-Asn-Pro-Ala-(Cyt)-Gly-Lys-Asn-Tyr-Ser-(Cyt)-NH² |
| 64^{1,2} | Ac-(Cyt)-(Cyt)-His-Pro-Ala-(Cyt)-Gly-Arg-His-Tyr-Ser-(Cyt)-NH₂ |
| 65^{1,2} | Ac-(Cyt)-(Cyt)-Asn-Pro-Ala-(Cyt)-Gly-Lys-Asn-Tyr-Lys-(Cyt)-NH₂ |
| 66^{2,3} | Ac-Cys-(Cyt)-Asn-Pro-Ala-Cys-Gly-Lys-Asn-Tyr-Ser-(Cyt)-NH₂ |
| 67^{2,3} | Ac-Cys-(Cyt)-His-Pro-Ala-Cys-Gly-Arg-His-Tyr-Ser-(Cyt)-NH₂ |
| 68^{2,3} | Ac-Cys-(Cyt)-Asn-Pro-Ala-Cys-Gly-Lys-Asn-Tyr-Lys-(Cyt)-NH₂ |
| 69^{1,4} | Ac-(Cyt)-Cys-Asn-Pro-Ala-(Cyt)-Gly-Lys-Asn-Tyr-Ser-Cys-NH₂ |
| 70^{1,4} | Ac-(Cyt)-Cys-His-Pro-Ala-(Cyt)-Gly-Arg-His-Tyr-Ser-Cys-NH₂ |
| 71^{1,4} | Ac-(Cyt)-Cys-Asn-Pro-Ala-(Cyt)-Gly-Lys-Asn-Tyr-Lys-Cys-NH₂ |
| 72^{1,4} | Ac-Arg-(Cyt)-Cys-His-Pro-Ala-(Cyt)-Gly-Lys-Asn-Tyr-Ser-Cys-NH₂ |
| 73^{2,3} | Ac-Arg-Cys-(Cyt)-His-Pro-Ala-Cys-Gly-Lys-Asn-Tyr-Ser-(Cyt)-NH₂ |
| 74^{3,4} | Ac-Arg-Cys-Cys-His-Pro-Ala-Cys-Gly-Lys-Asn-Tyr-Ser-Cys-NH₂ |
| 75^{1,2} | Ac-Arg-(Cyt)-(Cyt)-His-Pro-Ala-(Cyt)-Gly-Lys-Asn-Tyr-Ser-(Cyt)-NH₂ |
| 76^{2,3} | Ac-Cys-(Cyt)-His-Pro-Ala-Cys-Gly-Lys-Asn-Tyr-Ser-(Cyt)-NH₂ |
| 77^{1,2} | Ac-(Cyt)-(Cyt)-His-Pro-Ala-(Cyt)-Gly-Lys-Asn-Tyr-Ser-(Cyt)-NH₂ |
| 78^{3,4} | Ac-Cys-Cys-His-Pro-Ala-Cys-Gly-Lys-Asn-Tyr-Ser-Cys-NH₂ |
| 79^{1,4} | Ac-(Cyt)-Cys-His-Pro-Ala-(Cyt)-Gly-Lys-Asn-Tyr-Ser-Cys-NH₂ |
| 80^{3,4} | Ac-Cys-Cys-Lys-Pro-Ala-Cys-Gly-Arg-His-Tyr-Ser-Cys-NH₂ |
| 81^{3,4} | Ac-Cys-Cys-His-Pro-Ala-Cys-Gly-Arg-His-Tyr-Ser-Cys-NH₂ |
| 82^{3,4} | Ac-Cys-Cys-His-Pro-Ala-Cys-Gly-Arg-Asn-Tyr-Ser-Cys-NH₂ |
| 83^{3,4} | Ac-Cys-Cys-Lys-Pro-Ala-Cys-Gly-Arg-Asn-Tyr-Ser-Cys-NH₂ |
| 84^{1,4} | Ac-(Cyt)-Cys-His-Pro-Ala-(Cyt)-Gly-Arg-Asn-Tyr-Ser-Cys-NH₂ |
| 85^{3,4} | Ac-Cys-Cys-His-Pro-Ala-Cys-Gly-Lys-His-Tyr-Ser-Cys-NH₂ |
| 86^{3,4} | Ac-Cys-Cys-His-Pro-Ala-Cys-Gly-Arg-Lys-Tyr-Ser-Cys-NH₂ |
| 87^{1,2} | Ac-(Cyt)-(Cyt)-Lys-Pro-Ala-(Cyt)-Gly-Arg-His-Tyr-Ser-(Cyt)-NH₂ |
| 88^{1,4} | Ac-(Cyt)-Cys-His-Pro-Ala-(Cyt)-Gly-Lys-His-Tyr-Ser-Cys-NH₂ |
| 89^{3,4} | H-Cys-Cys-His-Pro-Ala-Cys-Gly-Lys-His-Tyr-Ser-Cys-NH₂ |
| 90^{3,4} | Ac-Cys-Cys-Asn-Pro-Ala-Cys-Gly-Arg-His-Tyr-Ser-Cys-NH₂ |
| 91^{3,4} | Ac-Cys-Cys-Asn-Pro-Ala-Cys-Gly-Arg-Asn-Tyr-Ser-Cys-NH₂ |
| 92^{1,4} | Ac-(Cyt)-Cys-Asn-Pro-Ala-(Cyt)-Gly-Arg-His-Tyr-Ser-Cys-NH₂ |
| 93^{3,4} | H-Cys-Cys-Asn-Pro-Ala-Cys-Gly-Arg-Asn-Tyr-Ser-Cys-NH₂ |
| 94^{3,4} | Ac-Cys-Cys-Lys-Pro-Gly-Cys-Gly-Arg-His-Tyr-Ser-Cys-NH₂ |
| 95^{3,4} | Ac-Cys-Cys-Asn-Pro-Ala-Cys-Gly-Lys-His-Tyr-Ser-Cys-NH₂ |
| 96^{3,4} | Ac-Cys-Cys-Lys-Pro-Ala-Cys-Gly-Lys-His-Tyr-Ser-Cys-NH₂ |
| 97^{3,4} | H-Cys-Cys-Asn-Pro-Ala-Cys-Gly-Lys-His-Tyr-Ser-Cys-NH₂ |
| 98^{3,4} | Ac-Cys-Cys-Asn-Pro-Ala-Cys-Gly-His-Lys-Tyr-Ser-Cys-NH₂ |
| 99^{1,4} | Ac-(Cyt)-Cys-Asn-Pro-Ala-(Cyt)-Gly-Lys-His-Tyr-Ser-Cys-NH₂ |

¹Comprises a cystathionine (Cyt-Cyt) linkage at (Xaa3-Xaa8);
²comprises a cystathionine (Cyt-Cyt) linkage at (Xaa4-Xaa14);
³comprises a disulfide (Cys-Cys) linkage at (Xaa3-Xaa8);
⁴comprises a disulfide (Cys-Cys) linkage at (Xaa4-Xaa14);
⁵comprises a Sec-Sec linkage at (Xaa3-Xaa8);
⁶comprises a Sec-Sec linkage at (Xaa4-Xaa14); all referring to Xaa1-Xaa14 numbering provided herein.

Unless otherwise indicated in the table, a peptide listed in Table 1 can comprise all L-amino acids or all D-amino acids.

Constraining Structures

In some embodiments, the peptide antagonist of the present disclosure comprises a constraining structure including, but not limited to, a linkage, bridge or any means of ligation between residues at two positions. In some embodiments, the peptide is constrained by its ends or at positions within the peptide, or both. In some embodiments, the constraining structure influences a peptide antagonist property, e.g., a pharmacokinetic property (including but not limited to absorption, bioavailability, distribution, metabolism, and excretion), a pharmacodynamic property (including but not limited to: receptor binding characteristics, e.g., binding half-life; postreceptor effects; and chemical interactions), enhanced activity (e.g., represented by IC₅₀), stability (e.g., represented by half-life), solubility (e.g., in a formulation), or permeability (e.g., permeability of the skin by a formulation containing the peptide antagonist). In certain embodiments, the constraining structure enhances stability of the peptide antagonist. In certain embodiments, the constraining structure enhances permeability through the skin of the peptide antagonist. In certain embodiments, the constraining structure enhances solubility in a formulation, e.g., a topical formulation, of the peptide antagonist.

In embodiments, a peptide antagonist that is constrained as described herein is referred to as a macrocyclic peptide or structure. A macrocyclic peptide refers to a closed-ring structure of a linear peptide intramolecularly formed by linkage between two positions in the peptide, referred to as linkage amino acids, linkage amino acid derivatives, linkage molecule, linkage moiety, linkage residue, linkage entity, or the like, as appropriate. The two linkage amino acids, linkage amino acid derivatives, linkage molecules, linkage moieties, linkage residues, or linkage entities are separated from each other by two or more amino acid residues, bound to each other directly, bound via a linker, or the like.

In embodiments, a linkage of a peptide antagonist of the disclosure is formed by two linkage amino acids, linkage amino acid derivatives, linkage molecules, linkage moieties, linkage residues, or linkage entities bound to each other by, e.g., a disulfide bond, a peptide bond, an alkyl bond, an alkenyl bond, an ester bond, a thioester bond, an ether bond, a thioether bond, a phosphonate ether bond, an azo bond, a C—S—C bond, a C═N—C bond, a C═N—C bond, an amide bond, a lactam bridge, a carbamoyl bond, an urea bond, a thiourea bond, an amine bond, a thioamide bond, or the like. The macrocyclization may be formed by a bond between an N-terminal amino acid and a C-terminal amino acid of a peptide, by a bond between a terminal amino acid and a non-terminal amino acid, or by a bond between non-terminal amino acids.

For convenience, reference to a specific amino acid involved in a linkage can use the nomenclature for the unlinked amino acid (e.g., the structure it may have prior to formation of a linkage). It is also understood that certain linkages, e.g., synthetic linkages, may not be formed by connecting two amino acids or derivatives as commonly referenced in the art. Therefore, references to linked amino acids herein may use the most closely approximating language to describe each involved chemical entity at a given residue position in the peptide antagonist. Correspondingly, linked entities in the peptide sequence, e.g., Xaa3, Xaa4, Xaa8, and Xaa14, may be referred to as linked amino acids, although they are not amino acids as commonly referenced in the art. In some embodiments, Xaa3 and Xaa8, and Xaa4 and Xaa14, when linked entities (e.g., forming an Xaa3-Xaa8 linkage and an Xaa4-Xaa14 linkage), can be referred to as linked (or linkage-forming) amino acids, linked (or linkage-forming) amino acid derivatives, linked (or linkage-forming) molecules, linked (or linkage-forming) moieties, linked (or linkage-forming) residues, or linked (or linkage-forming) entities in the alternative. These terms can be used to refer to amino acids, molecules, moieties, residues, or entities present at any of Xaa3, Xaa4, Xaa8, or Xaa14, in the alternative, either when linked or unlinked. For example, when not linked but intended to be linked in a peptide antagonist of the disclosure, two linkage amino acids also can be referred to as linked (or linkage-forming) amino acids, linked (or linkage-forming) amino acid derivatives, linked (or linkage-forming) molecules, linked (or linkage-forming) moieties, linked (or linkage-forming) residues, or linked (or linkage-forming) entities in the alternative. When linked, two linkage amino acids can be referred to as linked (or linkage-forming) amino acids, linked (or linkage-forming) amino acid derivatives, linked (or linkage-forming) molecules, linked (or linkage-forming) moieties, linked (or linkage-forming) residues, or linked (or linkage-forming) entities, in the alternative. When not linked and not intended to be linked, two amino acids can be referred to as unlinked (or non-linkage forming) amino acids, unlinked (or non-linkage forming) amino acid derivatives, unlinked molecules, unlinked moieties, unlinked residues, or unlinked entities. In some embodiments, each residue at a non-linked amino acid position in a peptide antagonist of the disclosure can be referred to as an amino acid, amino acid derivative, molecule, moiety, residue or entity, or as an unlinked (or non-linkage forming) amino acid, unlinked (or non-linkage forming) amino acid derivative, unlinked (or non-linkage forming) molecule, unlinked (or non-linkage forming) moiety, unlinked (or non-linkage forming) residue or unlinked (or non-linkage forming) entity.

Any constraining structure known to those of skill in the art is contemplated for linking the residues. Examples of constraining structures and their respective linkage residues include, but are not limited to linkages or bridges selected from: a disulfide bridge (e.g., a Cys-Cys linkage, wherein each linkage amino acid is a Cys); a Sec-Sec linkage (selenocysteine linkage, wherein each linkage amino acid is a selenocysteine); a cystathionine linkage or bridge (e.g., Ser-Homocysteine linkage), also referred to herein as Cyt-Cyt (e.g., CH₂—CH₂—S—CH₂); a lactam bridge (e.g., Asp-Lys or Glu-Lys linkage), a thioether linkage (e.g., a lanthionine linkage, including but not limited to Cys-dehydroalanine or methyl variant), and a dicarba linkage (e.g., a linkage of an olefin-containing amino acid, e.g., allyl glycine or prenyl glycine). In some embodiments, a linkage is selected from: a disulfide bridge having linkage residues Cys-Cys; a selenocysteine linkage having linkage residues Sec-Sec; a cystathionine linkage having linkage residues Ser-Homocysteine; a lactam bridge having residues Asp-Lys or Glu-Lys; a lanthionine linkage having linkage residues Cys-dehydroalanine or a methyl variant, and a dicarba linkage having linkage residues allyl glycine or prenyl glycine. In embodiments, linkage amino acid, linkage amino acid derivative, linkage molecule, linkage moiety, linkage residue, or linkage entity is selected from Cys, Sec, Ser, Homocysteine, Asp, Lys, Glu, dehydroalanine, or an olefin containing amino acid (e.g., allyl glycine or prenyl glycine).

In some embodiments, each of the Xaa3-Xaa8 and the Xaa4-Xaa14 linkage of a peptide antagonist of the disclosure is a linkage that is independently selected from: a disulfide bridge formed by two Cys linkage residues, a Sec-Sec linkage formed by two selenocysteine linkage residues, a cystathionine linkage formed by Ser and homocysteine linkage residues, a lactam bridge formed by Asp and Lys linkage residues or Glu and Lys linkage residues, a thioether linkage that is a lanthionine linkage formed by Cys and dehydroalanine or methyl variant residues, a dicarba linkage formed by olefin-containing linkage residues, e.g., an allyl glycine or prenyl glycine linkage residue, or any of these linkages formed by linkage residues as known and described in the art. In some embodiments, the Xaa3-Xaa8 or Xaa4-Xaa14 linkages are the same as one another, or different.

(See, e.g., Knerr et al., 2011, “Synthesis and activity of thioether-containing analogues of the complement inhibitor compstatin,” ACS Chem Biol. 6(7): 753-760; DiMarco et al., 2006, “Discovery of novel, highly potent and selective b-hairpin mimetic CXCR4 inhibitors with excellent anti-HIV activity and pharmacokinetic profiles,” Bioorganic & Medicinal Chemistry 14: 8396-8404; Dekan et al., 2011, “α-Conotoxin ImI incorporating stable cystathionine bridges maintains full potency and identical three-dimensional structure,” J. Am. Chem. Soc. 2011, 133: 15866-15869; Nguyen and Wong, 2017, “Making circles: recent advance in chemical and enzymatic approaches in peptide macrocyclization,” Journal of Biochemistry and Chemical Sciences 1(1): 1-13; Tam and Wong, 2012, “Chemical Synthesis of Circular Proteins,” The Journal of Biological Chemistry 287 (32): 27020-27025, each incorporated herein by reference in its entirety.) In some embodiments, any appropriate constraining structure resulting from the use of linkage residues as known in the art is contemplated for use in a peptide antagonist of the disclosure.

In some embodiments, a particular constraining structure is selected based on its resistance to degradation, e.g., degradation caused by the reduction of a disulfide bond constraining structure. In some embodiments, the peptide antagonist comprises a constraining structure that resists degradation by reduction. For example, in a reducing environment a disulfide bond may be susceptible to degradation and a resulting loss of activity or other desired peptide antagonist property. In some embodiments, a cystathione linkage or a linkage of at least two C₁-C₆ heterocycloalkyl rings confers increased stability relative to a disulfide bond.

In some embodiments, two amino acids in a chain are joined by a linkage to create a macrocyclic ring structure. In some embodiments, a linkage mimics a hairpin turn in a peptide. In some embodiments, linkages comprise covalent bonds between canonical or non-canonical amino acids such as cystathionine linkages, lactam bridges, or thioether bridges (e.g., a lanthionine linkage). In some embodiments, a linkage comprises a dipeptide. In some embodiments, a linkage comprises covalent bonds between canonical or non-canonical acid amino acids such as lanthionine or methyllanthionine linkages. In some embodiments, a linkage comprises at least one aromatic or non-aromatic ring. In some embodiments, a linkage comprises at least one cycloalkyl ring. In some embodiments, a linkage comprises at least one heterocyclic ring. In some embodiments, a linkage comprises at least two heterocyclic rings. In some embodiments, a linkage comprises at least one nitrogen-containing heterocycloalkyl ring.

In some embodiments, a linkage comprises the structure

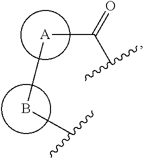

wherein A and B are heterocyclic rings. In some embodiments, a linkage comprises the structure

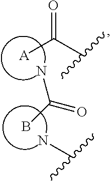

wherein A and B are heterocyclic rings.

In some embodiments, a linkage comprises pyrrolidine, piperidine, dehydropyrrolidine, dehydropiperidine, aziridine, azetidine, oxazolidine, or thiazolidine. In some embodiments, a linkage comprises two C₁-C₆ heterocycloalkyl rings. In some embodiments, a linkage comprises at least one five-membered heterocycloalkyl ring. In some embodiments, a linkage comprises at least one six-membered heterocycloalkyl ring. In some embodiments, a linkage comprises two five-membered heterocycloalkyl rings. In some embodiments, a linkage comprises two five-membered heterocycloalkyl rings, wherein each ring comprises at least one nitrogen atom. In some embodiments, a linkage comprises two five-membered heterocycloalkyl rings, wherein at least one ring comprises at least one nitrogen atom. In some embodiments, a linkage comprises two six-membered heterocycloalkyl rings. In some embodiments, the linkage comprises two C₁-C₆ heterocycloalkyl rings connected by an amide bond. In some embodiments, the linkage comprises two C₁-C₆ heterocycloalkyl rings connected by —C(═O)NH—. In some embodiments, a linkage comprises two pyrrolidine rings. In some embodiments, a linkage comprises at least one non-canonical amino (unnatural) acid residue. In some embodiments, a linkage comprises two amino acids (canonical or non-canonical), wherein a first amino acid has the (S) configuration at the alpha position, and the second amino acid has the (R) configuration at the alpha position. In some embodiments, a linkage comprises two amino acids (canonical or non-canonical) connected by a peptide bond. In some embodiments, a linkage comprises two proline residues (diproline linkage). In some embodiments, a linkage comprises two proline residues connected by a peptide bond. In some embodiments, a linkage comprises a D-proline and an L-proline (D-proline-L-proline or L-proline-D-proline).

In some embodiments, a linkage comprises a D-proline and an L-proline, or derivatives thereof. In some embodiments, such derivatives comprise substitutions to the pyrrolidine ring of a proline. In some embodiments, a linkage comprises a non-canonical amino acid residue selected from 3-fluoroproline, 4-fluoroproline, 3-hydroxyproline, 4-hydroxyproline, 3-aminoproline, 4-aminoproline, 3,4-dehydroproline, aziridine-2-carboxylic acid, azetidine-2-carboxylic acid, pipecolic acid, 4-oxa-proline, 3-thiaproline, or 4-thiaproline. In some embodiments, a linkage comprises two amino acids selected from proline, 3-fluoroproline, 4-fluoroproline, 3-hydroxyproline, 4-hydroxyproline, 3-aminoproline, 4-aminoproline, 3,4-dehydroproline, aziridine-2-carboxylic acid, azetidine-2-carboxylic acid, pipecolic acid, 4-oxa-proline, 3-thiaproline, or 4-thiaproline.

In some embodiments, a linkage comprises covalent bonds between canonical or non-canonical amino acids lactam bridges. In some embodiments, a linkage comprises the structure:

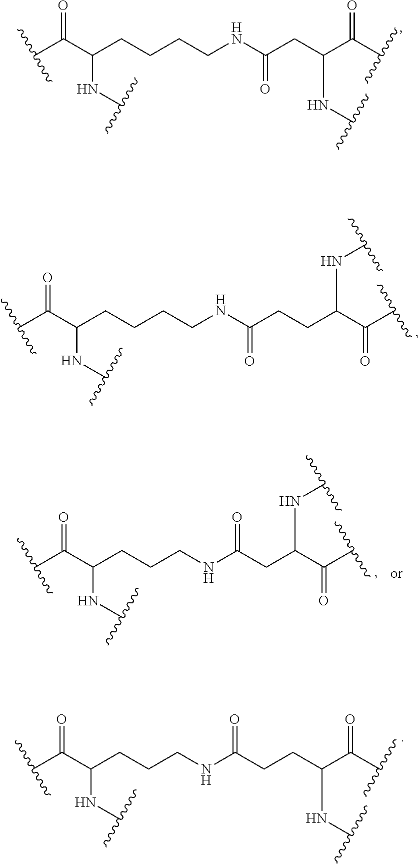 [SMILES: *NC(CCCCNC(=O)CC(N*)C(*)=O)C(*)=O ; *NC(CCCCNC(=O)CCC(N*)C(*)=O)C(*)=O ; *NC(CCCNC(=O)CC(N*)C(*)=O)C(*)=O ; *NC(CCCNC(=O)CCC(N*)C(*)=O)C(*)=O]

In some embodiments, a linkage comprises covalent bonds between canonical or non-canonical amino acids thioether bridges. In some embodiments, a linkage comprises the structure

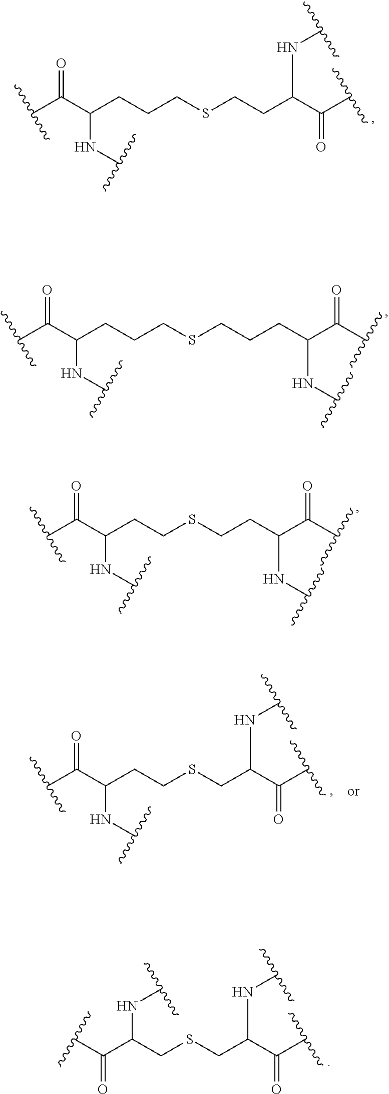 [SMILES: *NC(CCCSCCC(N*)C(*)=O)C(*)=O ; *NC(CCCSCCCC(N*)C(*)=O)C(*)=O ; *NC(CCSCCC(N*)C(*)=O)C(*)=O ; *NC(CCSCC(N*)C(*)=O)C(*)=O ; *NC(CSCC(N*)C(*)=O)C(*)=O]

These and similar constraining structures can be used to link residues at terminal and/or nonterminal positions in the peptide. In some embodiments, Xaa3 and Xaa8 of a peptide antagonist of the disclosure are linked. In some embodiments, Xaa4 and Xaa14 of a peptide antagonist of the disclosure are linked. In some embodiments, Xaa3 and Xaa8, and Xaa4 and Xaa14, of a peptide antagonist of the disclosure are linked.

### Linkage Spacing

In some embodiments, a constraining structure as described herein is selected based on the resulting spatial separation between the constrained residues. In some embodiments, the spatial separation influences a peptide antagonist property as described above. A peptide antagonist of the disclosure can comprise a constraining structure conferring a spatial separation of about 3.5 to about 10 Ångströms between alpha-carbons of the two linked amino acid residues, or between the geometrical centers of the two linked residues (e.g., amino acid derivatives). In some embodiments, the spatial separation between the alpha-carbons of the two linked amino acid residues, or the spatial separation between the geometrical centers of the two linked residues, is about 3.5 Ångströms to about 10 Ångströms. In some embodiments, the spatial separation between the alpha-carbons of the two linked amino acid residues, or the spatial separation between the geometrical centers of the two linked residues, is at least about 3.5 Ångströms. In some embodiments, the spatial separation between the alpha-carbons of the two linked amino acid residues, or the spatial separation between the geometrical centers of the two linked residues, is at most about 10 Ångströms. In some embodiments, the spatial separation between the alpha-carbons of the two linked amino acid residues, or the spatial separation between the geometrical centers of the two linked residues, is about 3.5 Ångströms to about 4.5 Ångströms, about 3.5 Ångströms to about 5 Ångströms, about 3.5 Ångströms to about 5.5 Ångströms, about 3.5 Ångströms to about 6 Ångströms, about 3.5 Ångströms to about 6.5 Ångströms, about 3.5 Ångströms to about 7 Ångströms, about 3.5 Ångströms to about 7.5 Ångströms, about 3.5 Ångströms to about 8 Ångströms, about 3.5 Ångströms to about 8.5 Ångströms, about 3.5 Ångströms to about 9 Ångströms, about 3.5 Ångströms to about 10 Ångströms, about 4.5 Ångströms to about 5 Ångströms, about 4.5 Ångströms to about 5.5 Ångströms, about 4.5 Ångströms to about 6 Ångströms, about 4.5 Ångströms to about 6.5 Ångströms, about 4.5 Ångströms to about 7 Ångströms, about 4.5 Ångströms to about 7.5 Ångströms, about 4.5 Ångströms to about 8 Ångströms, about 4.5 Ångströms to about 8.5 Ångströms, about 4.5 Ångströms to about 9 Ångströms, about 4.5 Ångströms to about 10 Ångströms, about 5 Ångströms to about 5.5 Ångströms, about 5 Ångströms to about 6 Ångströms, about 5 Ångströms to about 6.5 Ångströms, about 5 Ångströms to about 7 Ångströms, about 5 Ångströms to about 7.5 Ångströms, about 5 Ångströms to about 8 Ångströms, about 5 Ångströms to about 8.5 Ångströms, about 5 Ångströms to about 9 Ångströms, about 5 Ångströms to about 10 Ångströms, about 5.5 Ångströms to about 6 Ångströms, about 5.5 Ångströms to about 6.5 Ångströms, about 5.5 Ångströms to about 7 Ångströms, about 5.5 Ångströms to about 7.5 Ångströms, about 5.5 Ångströms to about 8 Ångströms, about 5.5 Ångströms to about 8.5 Ångströms, about 5.5 Ångströms to about 9 Ångströms, about 5.5 Ångströms to about 10 Ångströms, about 6 Ångströms to about 6.5 Ångströms, about 6 Ångströms to about 7 Ångströms, about 6 Ångströms to about 7.5 Ångströms, about 6 Ångströms to about 8 Ångströms, about 6 Ångströms to about 8.5 Ångströms, about 6 Ångströms to about 9 Ångströms, about 6 Ångströms to about 10 Ångströms, about 6.5 Ångströms to about 7 Ångströms, about 6.5 Ångströms to about 7.5 Ångströms, about 6.5 Ångströms to about 8 Ångströms, about 6.5 Ångströms to about 8.5 Ångströms, about 6.5 Ångströms to about 9 Ångströms, about 6.5 Ångströms to about 10 Ångströms, about 7 Ångströms to about 7.5 Ångströms, about 7 Ångströms to about 8 Ångströms, about 7 Ångströms to about 8.5 Ångströms, about 7 Ångströms to about 9 Ångströms, about 7 Ångströms to about 10 Ångströms, about 7.5 Ångströms to about 8 Ångströms, about 7.5 Ångströms to about 8.5 Ångströms, about 7.5 Ångströms to about 9 Ångströms, about 7.5 Ångströms to about 10 Ångströms, about 8 Ångströms to about 8.5 Ångströms, about 8 Ångströms to about 9 Ångströms, about 8 Ångströms to about 10 Ångströms, about 8.5 Ångströms to about 9 Ångströms, about 8.5 Ångströms to about 10 Ångströms, or about 9 Ångströms to about 10 Ångströms. In some embodiments, the spatial separation between the alpha-carbons of the two linked amino acid residues, or the spatial separation between the geometrical centers of the two linked residues, is about 3.5 Ångströms, about 4.5 Ångströms, about 5 Ångströms, about 5.5 Ångströms, about 6 Ångströms, about 6.5 Ångströms, about 7 Ångströms, about 7.5 Ångströms, about 8 Ångströms, about 8.5 Ångströms, about 9 Ångströms, or about 10 Ångströms. In embodiments, a specific spatial separation is achieved using a linker or spacer molecule, as known in the art.

### Amino Acid Derivatives

The present disclosure contemplates the use of an amino acid derivative or analog of any amino acid in any of the peptide antagonists of the disclosure. In some embodiments, amino acid modifications can be made chemically using any known method. Selective protein modifications are described in the literature, e.g., by Spicer and Davis, 2014, “Selective chemical protein modification,” Nature Communications 5: 4740, incorporated herein by reference.

In some embodiments, an amino acid derivative is a non-canonical amino acid. In some embodiments, a non-canonical amino acid has an (S) configuration at the alpha position. In some embodiments, a non-canonical amino acid has an (R) configuration at the alpha position. In some embodiments, a non-canonical amino acid is an alpha amino acid. In some embodiments, a non-canonical amino acid is a beta or gamma amino acid. In some embodiments, a non-canonical amino acid is selected from the group consisting of: an aromatic side chain amino acid; a non-aromatic side chain amino acid; an aliphatic side chain amino acid; a side chain amide amino acid; a side chain ester amino acid; a heteroaromatic side chain amino acid; a side chain thiol amino acid; a beta amino acid; and a backbone-modified amino acid. In some embodiments, a non-canonical amino acid is a derivative of tyrosine, histidine, tryptophan, or phenylalanine. In some embodiments, a derivative of an amino acid comprises an ester, amide, disulfide, carbamate, urea, phosphate, ether of the amino acid. In some embodiments, a non-aromatic side chain amino acid is a derivative of serine, threonine, cysteine, methionine, arginine, asparagine, glutamine, aspartic acid, glutamic acid, lysine, proline, glycine, alanine, valine, isoleucine, or leucine. In some embodiments, a non-canonical amino acid is selected from the group consisting of 2-aminoadipic acid; 3-aminoadipic acid; beta-alanine; beta-aminoproprionic acid; 2-aminobutyric acid; 4-aminobutyric acid; piperidinic acid; 6-aminocaproic acid; 2-aminoheptanoic acid; 2-aminoisobutyric acid; 3-aminoisobutyric acid; 2-aminopimelic acid; 2,4-diaminobutyric acid; desmosine; 2,2′-diaminopimelic acid; 2,3-diaminoproprionic acid; N-ethylglycine; N-ethylasparagine; hydroxylysine; allo-hydroxylysine; 3-hydroxyproline; 4-hydroxyproline; isodesmosine; allo-isoleucine; N-methylglycine; sarcosine; n-methylisoleucine; 6-N-methyllysine; N-methylvaline; norvaline; norleucine; and ornithine. In some embodiments, a non-canonical amino acid is a proline derivative. In some embodiments, a proline derivative is 3-fluoroproline, 4-fluoroproline, 3-hydroxyproline, 4-hydroxyproline, 3-aminoproline, 4-aminoproline, 3,4-dehydroproline, aziridine-2-carboxylic acid, azetidine-2-carboxylic acid, pipecolic acid, 4-oxa-proline, 3-thiaproline, or 4-thiaproline. In some embodiments, a non-canonical amino acid comprises a lipid.

In some embodiments, a peptide antagonist of the disclosure comprises one or more amino acid derivative or analog, e.g., as known to those of skill in the art and described in the literature or herein. In some embodiments, a peptide antagonist of the disclosure comprises 1, 2, 3, 4, 5, 6, 7, 8, 9, 10, 11, 12, 13, 14, 1-2, 1-3, 1-4, 1-5, 1-6, 1-7, 1-8, 1-9, 1-10, 1-11, 1-12, or 1-13 amino acid derivatives.

In some embodiments, each amino acid derivative present in a peptide antagonist of the disclosure is a non-canonical amino acid independently selected from the group consisting of: an aromatic side chain amino acid; a non-aromatic side chain amino acid; an aliphatic side chain amino acid; a side chain amide amino acid; a side chain ester amino acid; a heteroaromatic side chain amino acid; a side chain thiol amino acid; a beta amino acid; and a backbone-modified amino acid, selected from e.g., the non-canonical amino acids described herein or known in the art and described in the published literature.

In some embodiments, the peptide antagonist comprises one or more amino acids that have the D-amino acid configuration, and the remaining amino acids in the peptide have the L-amino acid configuration.

In some embodiments, a non-canonical amino acid is a proline derivative. In some embodiments, a proline derivative comprises one or more substitutions on the pyrrolidine ring. In some embodiments, a proline derivative comprises one or more substitutions on the pyrrolidine ring, wherein the substitutions comprise halogen, alkoxy, amino, hydroxyl, alkyl (methyl, ethyl), thiol, or alkylthio. In some embodiments, a proline derivative comprises one or more substitutions on the pyrrolidine ring, wherein the substitutions comprise halogen, or alkyl (methyl, ethyl). In some embodiments, a proline derivative comprises one or more substitutions on the pyrrolidine ring, wherein the substitutions comprise halogen. In some embodiments, a proline derivative comprises one or more substitutions on the pyrrolidine ring, wherein the substitutions comprise alkoxy, hydroxyl, amino. In some embodiments, a proline derivative comprises one or more substitutions on the pyrrolidine ring, wherein the substitutions comprise halogen, alkoxy, alkyl (methyl, ethyl), thiol, or alkylthio.

### N-Terminal Modification of the Peptide Antagonist

In some embodiments, the N-terminus amino group of the peptide antagonist of the disclosure is modified (N-terminal modifications). In some embodiments, the N-terminus of the peptide antagonist is not modified with an additional amino acid or amino acid derivative. In some embodiments, an unmodified N terminus comprises hydrogen. In some embodiments, an N-terminal modification comprises C₁-C₆ acyl, C₁-C₈ alkyl, C₆-C₁₂ aralkyl, C₅-C₁₀ aryl, C₄-C₈ heteroaryl, formyl, or a lipid. In some embodiments, an N-terminal modification comprises C₆-C₁₂ aralkyl. In some embodiments, an N-terminal modification comprises C₁-C₆ acyl. In some embodiments, an N-terminal modification comprises acetyl (Ac). In some embodiments, an N-terminal modification comprises C₁-C₆ alkyl. In some embodiments, an N-terminal modification comprises methyl, ethyl, propyl, or tert-butyl. In some embodiments, an N-terminal modification comprises C₁-C₆ aralkyl. In some embodiments, an N-terminal modification comprises benzyl. In some embodiments, an N-terminal modification comprises formyl. In some embodiments, a peptide described herein, e.g., any peptide having an amino acid sequence as listed in Table 2 (irrespective of the N-terminus shown in the table), has any of these N-terminal modification or an unmodified N-terminus.

### C-Terminal Modification of the Peptide Antagonist

In some embodiments, the C-terminus acid group of the peptide antagonist of the disclosure is modified (C-terminal modifications). In some embodiments, the C-terminus is not modified with an additional amino acid or amino acid derivative. In some embodiments, the C-terminus is not modified with a glycine residue. In some embodiments, an unmodified C terminus comprises—OH. In some embodiments, a C-terminal modification comprises an amino group, wherein the amino group is optionally substituted. In some embodiments, a C-terminal modification comprises an amino group, wherein the amino group is unsubstituted (—NH₂). In some embodiments, a C-terminal modification comprises an amino group, wherein the amino group is substituted. In some embodiments, a C-terminal modification comprises—NH₂, -amino-acyl, -amino-C₁-C₈ alkyl, -amino-C₆-C₁₂-aralkyl, -amino-C₅-C₁₀ aryl, or -amino-C₄-C₈ heteroaryl, -amino-C₄-C₈ heteroaryl, or —O—(C₁-C₈ alkyl). In some embodiments, a C-terminal modification comprises -amino-C₆-C₁₂-aralkyl. In some embodiments, a C-terminal modification comprises—O—(C₁-C₈ alkyl). In some embodiments, a C-terminal modification comprises -amino-C₆-C₁₂-aralkyl. In some embodiments, a C-terminal modification comprises—NH—CH₂Phenyl. In some embodiments, a C-terminal modification comprises —OEt. In some embodiments, a C-terminal modification comprises—OMe. In some embodiments, a peptide described herein, e.g., any peptide having an amino acid sequence as listed in Table 2 (irrespective of the C-terminus shown in the table), has any of these C-terminal modifications or an unmodified C-terminus.

In some embodiments, both the N-terminus amino group and the C-terminus acid group of the peptide antagonist of the disclosure are modified. In some embodiments, a peptide described herein, e.g., any peptide having an amino acid sequence as listed in Table 2 (irrespective of the N- and C-termini shown in the table), has N- and C-termini independently selected from any described herein. In some embodiments, a peptide described herein, e.g., any peptide having an amino acid sequence as listed in Table 2 (irrespective of the N- and C-termini shown in the table), has N- and C-termini independently selected from: Ac, NH₂, and H.

### Concentrations of Peptide Antagonist in the Composition

In some embodiments, the peptide antagonist is present in the vesicle composition in an amount of about 0.1 mg/mL to about 50 mg/mL. In some embodiments, the peptide antagonist is present in the vesicle composition in an amount of about 0.1 mg/mL to about 0.5 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL, about 0.5 mg/mL to about 1 mg/mL, about 0.5 mg/mL to about 2 mg/mL, about 0.5 mg/mL to about 3 mg/mL, about 0.5 mg/mL to about 4 mg/mL, about 0.5 mg/mL to about 5 mg/mL, about 0.5 mg/mL to about 10 mg/mL, about 0.5 mg/mL to about 20 mg/mL, about 0.5 mg/mL to about 50 mg/mL, about 1 mg/mL to about 2 mg/mL, about 1 mg/mL to about 3 mg/mL, about 1 mg/mL to about 4 mg/mL, about 1 mg/mL to about 5 mg/mL, about 1 mg/mL to about 10 mg/mL, about 1 mg/mL to about 20 mg/mL, about 1 mg/mL to about 50 mg/mL, about 2 mg/mL to about 3 mg/mL, about 2 mg/mL to about 4 mg/mL, about 2 mg/mL to about 5 mg/mL, about 2 mg/mL to about 10 mg/mL, about 2 mg/mL to about 20 mg/mL, about 2 mg/mL to about 50 mg/mL, about 3 mg/mL to about 4 mg/mL, about 3 mg/mL to about 5 mg/mL, about 3 mg/mL to about 10 mg/mL, about 3 mg/mL to about 20 mg/mL, about 3 mg/mL to about 50 mg/mL, about 4 mg/mL to about 5 mg/mL, about 4 mg/mL to about 10 mg/mL, about 4 mg/mL to about 20 mg/mL, about 4 mg/mL to about 50 mg/mL, about 5 mg/mL to about 10 mg/mL, about 5 mg/mL to about 20 mg/mL, about 5 mg/mL to about 50 mg/mL, about 10 mg/mL to about 20 mg/mL, about 10 mg/mL to about 50 mg/mL, or about 20 mg/mL to about 50 mg/mL. In some embodiments, the peptide antagonist is present in the vesicle composition in an amount of about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the peptide antagonist is present in the vesicle composition in an amount of at least about 0.1 mg/mL, about 0.5 mg/mL, about 1 mg/mL, about 2 mg/mL, about 3 mg/mL, about 4 mg/mL, about 5 mg/mL, about 10 mg/mL, or about 20 mg/mL. In some embodiments, the peptide antagonist is present in the vesicle composition in an amount of at most about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the peptide antagonist is present in the composition in an amount of about 1 mg/mL, about 2 mg/mL, about 3 mg/mL, about 4 mg/mL, or about 5 mg/mL.

### Vesicle Forming Lipids

In some embodiments, the vesicle composition comprises one or more vesicle forming lipids. The vesicle forming lipids act to encapsulate portions of the oil-in-water emulsions. In some embodiments, this allows the oil-in-water emulsion to remain stable for a period of time.

The vesicle forming lipids may be any suitable lipids for such a purpose. In some embodiments, the vesicle forming lipids comprise phospholipids, glycolipids, lecithins, ceramides, lysolecithin, lysophosphatidylethanolamine, phosphatidylserine, phosphatidylinositol, sphingomyelin, cardiolipin, phosphatidic acid, cerebroside, or any combination thereof. In some embodiments, the vesicle forming lipids comprise a combination of lipids.

In some embodiments, the vesicle forming lipids comprise phospholipids. In some embodiments, the phospholipids are naturally occurring, semisynthetic, or synthetically prepared, or a mixture thereof. In an embodiment, the phospholipids are one or more esters of glycerol with one or two (equal or different) residues of fatty adds and with phosphoric acid, wherein the phosphoric acid residue is in turn bound to a hydrophilic group, such as, for instance, choline (phosphatidylcholines—PC), serine (phosphatidylserines—PS), glycerol (phosphatidylglycerols—PG), ethanolamine (phosphatidylethanolamines—PE), or inositol (phosphatidylinositol). Esters of phospholipids with only one residue of fatty acid are generally referred to in the art as the “lyso” forms of the phospholipid or “lysophospholipids”. Fatty acids residues present in the phospholipids are in general long chain aliphatic acids, typically containing 12 to 24 carbon atoms, or 14 to 22 carbon atoms; the aliphatic chain may contain one or more unsaturations or is completely saturated. Examples of suitable fatty acids included in the phospholipids are, for instance, lauric acid, myristic acid, palmitic acid, stearic acid, arachidic acid, behenic acid, oleic acid, linoleic acid, and linolenic acid. Saturated fatty acids such as myristic acid, palmitic acid, stearic acid and arachidic acid may be employed.

In some embodiments, the phospholipid comprises one or more natural phospholipids. In some embodiments, the phospholipid comprises one or more semisynthetic phospholipids. In some embodiments, the semisynthetic phospholipids are the partially or fully hydrogenated derivatives of the naturally occurring lecithins. In some embodiments, the phospholipids include fatty acids di-esters of phosphatidylcholine, ethylphosphatidylcholine, phosphatidylglycerol, phosphatidic acid, phosphatidylethanolamine, phosphatidylserine or of sphingomyelin. In some embodiments, the phospholipids include hydrogenated phosphatidylcholine (e.g., Sunlipon 90H). In some embodiments, the phospholipids are, for instance, dilauroyl-phosphatidylcholine (DLPC), dimyristoyl-phosphatidylcholine (DMPC), dipalmitoyl-phosphatidylcholine (DPPC), diarachidoyl-phosphatidylcholine (DAPC), distearoyl-phosphatidylcholine (DSPC), dioleoyl-phosphatidylcholine (DOPC), 1,2Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), 1-palmitoyl-2-oleylphosphatidylcholine (POPC), 1-oleyl-2-palmitoyl-phosphatidylcholine (OPPC), dilauroylphosphatidylglycerol (DLPG) and its alkali metal salts, diarachidoylphosphatidylglycerol (DAPG) and its alkali metal salts, dimyristoylphosphatidylglycerol (DMPG) and its alkali metal salts, dipalmitoylphosphatidylglycerol (DPPG) and its alkali metal salts, distearoylphosphatidylglycerol (DSPG) and its alkali metal salts, dioleoyl-phosphatidylglycerol (DOPG) and its alkali metal salts, dimyristoyl phosphatidic acid DMPA) and its alkali metal salts, dipalmitoyl phosphatidic acid (DPPA) and its alkali metal salts, distearoyl phosphatidic acid (DSPA), diarachidoylphosphatidic acid (DAPA) and its alkali metal salts, dimyristoylphosphatidylethanolamine (DMPE), dipalmitoylphosphatidylethanolamine (DPPE), distearoyl phosphatidyl-ethanolamine (DSPE), dioleylphosphatidylethanolamine (DOPE), diarachidoylphosphatidylethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), and distearoylsphingomyelin (DSSP), dilauroyl-phosphatidylinositol (DLPI), diarachidoylphosphatidylinositol (DAPI), dimyristoylphosphatidylinositol (DMPI), dipalmitoylphosphatidylinositol (DPPI), distearoylphosphatidylinositol (DSPI), dioleoyl-phosphatidylinositol (DOPI).

In some embodiments, the vesicle forming lipids are present in an amount of about 0.5% to about 25% (w/w) of the composition. In some embodiments, the vesicle forming lipids are present in an amount of about 0.5% to about 2%, about 0.5% to about 5%, about 0.5% to about 8%, about 0.5% to about 10%, about 0.5% to about 12%, about 0.5% to about 15%, about 0.5% to about 20%, about 0.5% to about 25%, about 2% to about 5%, about 2% to about 8%, about 2% to about 10%, about 2% to about 12%, about 2% to about 15%, about 2% to about 20%, about 2% to about 25%, about 5% to about 8%, about 5% to about 10%, about 5% to about 12%, about 5% to about 15%, about 5% to about 20%, about 5% to about 25%, about 8% to about 10%, about 8% to about 12%, about 8% to about 15%, about 8% to about 20%, about 8% to about 25%, about 10% to about 12%, about 10% to about 15%, about 10% to about 20%, about 10% to about 25%, about 12% to about 15%, about 12% to about 20%, about 12% to about 25%, about 15% to about 20%, about 15% to about 25%, or about 20% to about 25% (w/w) of the composition. In some embodiments, the vesicle forming lipids are present in an amount of about 0.5%, about 2%, about 5%, about 8%, about 10%, about 12%, about 15%, about 20%, or about 25%. In some embodiments, the vesicle forming lipids are present in an amount of at least about 0.5%, about 2%, about 5%, about 8%, about 10%, about 12%, about 15%, or about 20% (w/w) of the composition. In some embodiments, the vesicle forming lipids are present in an amount of at most about 2%, about 5%, about 8%, about 10%, about 12%, about 15%, about 20%, or about 25% (w/w) of the composition.

In some embodiments, the vesicle forming lipids are present in an amount of about 5% to about 15% (w/w) of the composition. In some embodiments, the vesicle forming lipids are present in an amount of about 5% to about 8%, about 5% to about 9%, about 5% to about 10%, about 5% to about 11%, about 5% to about 12%, about 5% to about 13%, about 5% to about 14%, about 5% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 11% to about 12%, about 11% to about 13%, about 11% to about 14%, about 11% to about 15%, about 12% to about 13%, about 12% to about 14%, about 12% to about 15%, about 13% to about 14%, about 13% to about 15%, or about 14% to about 15%. In some embodiments, the vesicle forming lipids are present in an amount of about 5%, about 8%, about 9%, about 10%, about 11%, about 12%, about 13%, about 14%, or about 15% (w/w) of the composition. In some embodiments, the vesicle forming lipids are present in an amount of at least about 5%, about 8%, about 9%, about 10%, about 11%, about 12%, about 13%, or about 14% (w/w) of the composition. In some embodiments, the vesicle forming lipids are present in an amount of at most about 8%, about 9%, about 10%, about 11%, about 12%, about 13%, about 14%, or about 15% (w/w) of the composition.

In some embodiments, the composition comprises a short chain polyol. In some embodiments, the short chain polyol acts to enhance the stability of the resulting lipid vesicles. In some embodiments, the short chain polyol is a C₂-C₄ polyol comprising two or three alcohol groups. In some embodiments, the short chain polyol is propylene glycol. In some embodiments, the composition comprises propylene glycol.

In some embodiments, the propylene glycol is present in an amount of about 0.5% to about 25% (w/w) of the composition. In some embodiments, the propylene glycol is present in an amount of about 0.5% to about 2%, about 0.5% to about 5%, about 0.5% to about 8%, about 0.5% to about 10%, about 0.5% to about 12%, about 0.5% to about 15%, about 0.5% to about 20%, about 0.5% to about 25%, about 2% to about 5%, about 2% to about 8% about 2% to about 10%, about 2% to about 12%, about 2% to about 15%, about 2% to about 20%, about 2% to about 25%, about 5% to about 8%, about 5% to about 10%, about 5% to about 12%, about 5% to about 15%, about 5% to about 20%, about 5% to about 25%, about 8% to about 10%, about 8% to about 12%, about 8% to about 15%, about 8% to about 2000, about 8% to about 25%, about 10% to about 12%, about 10% to about 15%, about 10% to about 20%, about 10% to about 25%, about 12% to about 15%, about 12% to about 20%, about 12% to about 25%, about 15% to about 20%, about 15% to about 25%, or about 20% to about 25%. In some embodiments, the propylene glycol is present in an amount of about 0.5%, about 2% about 5%, about 8%, about 10%, about 12%, about 15%, about 20%, or about 25%. In some embodiments, the propylene glycol is present in an amount of at least about 0.5%, about 2%, about 5%, about 8%, about 10%, about 12%, about 15%, or about 20%. In some embodiments, the propylene glycol is present in an amount of at most about 2%, about 5% about 8%, about 10%, about 12%, about 15%, about 20%, or about 25%. In some embodiments, the propylene glycol is present in an amount of about 1% to about 10%. In some embodiments, the propylene glycol is present in an amount of about 1% to about 2%, about 1% to about 4%, about 1% to about 6%, about 1% to about 8%, about 1% to about 10%, about 2% to about 4%, about 2% to about 6%, about 2% to about 8%, about 2% to about 10%, about 4% to about 6%, about 4% to about 8%, about 4% to about 10%, about 6% to about 8%, about 6% to about 10%, or about 8% to about 10%. In some embodiments, the propylene glycol is present in an amount of about 1%, about 2%, about 4%, about 6%, about 8%, or about 10%. In some embodiments, the propylene glycol is present in an amount of at least about 1%, about 2%, about 4%, about 6%, or about 8%. In some embodiments, the propylene glycol is present in an amount of at most about 2%, about 4%, about 6%, about 8%, or about 10%. In some embodiments, propylene glycol is present in about the same amount as the vesicle forming lipid. In some embodiments, the ratio of propylene glycol to vesicle forming lipid in the composition is form about 2:1 to about 1:2 (w/w).

### Oil Phases

The lipid vesicle compositions provided herein comprise an oil-in-water emulsion. The oil component is selected such that the material is a liquid at operative temperatures (e.g., room temperature) and is non-miscible with water.

Any suitable oil may be used as the oil phase. In some embodiments, the oil comprises a naturally occurring oil. In some embodiments, the naturally occurring oil is derived from one or more plants or plant parts (e.g., seeds or nuts). In some embodiments, the oil is a naturally occurring oil such as olive oil, vegetable oil, sunflower oil, or other similar plant derived oil.

In some embodiments, the oil phase is selected from the group consisting of vegetable oils, mono-, di-, and triglycerides, silicone fluids, mineral oils, and combinations thereof.

In some embodiments, the oil comprises a silicon oil or derivative, such as dimethicone. In some embodiments, the oil silicon oil comprises a siloxane polymer. In some embodiments, the siloxane polymer comprises C₁-C₃ substituents. In some embodiments, the siloxane is polydimethylsiloxane (PDMS). In some embodiments, the oil is a mixture which comprises a silicon oil (e.g., dimethicone) as a smaller component. In some embodiments, the silicon oil is incorporated in order to enhance the feel of the resulting composition or as a moisturizer. In some embodiments, the oil comprises a silicon oil in an amount of up to about 5%, up to about 4%, up to about 3%, up to about 2%, or up to about 1%. In some embodiments, the silicon oil is present in an amount of from about 0.1% to about 2% (w/w) of the composition.

In some embodiments, the oils are present in an amount of about 1% to about 35% (w/w) of the composition. In some embodiments, the oils are present in an amount of about 1% to about 5%, about 1% to about 10%, about 1% to about 15%, about 1% to about 20%, about 1% to about 25%, about 1% to about 30%, about 1% to about 35%, about 5% to about 10%, about 5% to about 15%, about 5% to about 20%, about 5% to about 25%, about 5% to about 30%, about 5% to about 35%, about 10% to about 15%, about 10% to about 20%, about 10% to about 25%, about 10% to about 30%, about 10% to about 35%, about 15% to about 20%, about 15% to about 25%, about 15% to about 30%, about 15% to about 35%, about 20% to about 25%, about 20% to about 30%, about 20% to about 35%, about 25% to about 30%, about 25% to about 35%, or about 30% to about 35%. In some embodiments, the oils are present in an amount of about 1%, about 5%, about 10%, about 15%, about 20%, about 25%, about 30%, or about 35%. In some embodiments, the oils are present in an amount of at least about 1%, about 5%, about 10%, about 15%, about 20%, about 25%, or about 30%. In some embodiments, the oils are present in an amount of at most about 5%, about 10%, about 15%, about 20%, about 25%, about 30%, or about 35%. In some embodiments, the oils are present in an amount of about 5% to about 15%. In some embodiments, the oils are present in an amount of about 5% to about 8%, about 5% to about 9%, about 5% to about 10%, about 5% to about 11%, about 5% to about 12%, about 5% to about 13%, about 5% to about 14%, about 5% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 11% to about 12%, about 11% to about 13%, about 11% to about 14%, about 11% to about 15%, about 12% to about 13%, about 12% to about 14%, about 12% to about 15%, about 13% to about 14%, about 13% to about 15%, or about 14% to about 15%. In some embodiments, the oils are present in an amount of about 5%, about 8%, about 9% about 10%, about 11%, about 12%, about 13%, about 14%, or about 15%. In some embodiments, the oils are present in an amount of at least about 5%, about 8%, about 9% about 1000, about 11%, about 12%, about 13%, or about 14%. In some embodiments, the oils are present in an amount of at most about 8%, about 9%, about 10%, about 11%, about 12% about 13%, about 14%, or about 15%.

In some embodiments, the oil comprises one or more triglycerides. In some embodiments the triglyceride is a medium chain triglyceride. In some embodiments, the medium chain triglyceride comprises fatty acid esters having a chain length of C₆-C₁₂.

In some embodiments, the triglyceride is present in an amount of about 1% to about 35% (w/w) of the composition. In some embodiments, the triglyceride is present in an amount of about 1% to about 5%, about 1% to about 10%, about 1% to about 15%, about 1% to about 20%, about 1% to about 25%, about 1% to about 30%, about 1% to about 35%, about 5% to about 10%, about 5% to about 15%, about 5% to about 20%, about 5% to about 25%, about 5% to about 30%, about 5% to about 35%, about 10% to about 15%, about 10% to about 20%, about 10% to about 25%, about 10% to about 30%, about 10% to about 35%, about 15% to about 20%, about 15% to about 25%, about 15% to about 30%, about 15% to about 35%, about 20% to about 25%, about 20% to about 30%, about 20% to about 35%, about 25% to about 30%, about 25% to about 35%, or about 30% to about 35%. In some embodiments, the triglyceride is present in an amount of about 1%, about 5%, about 10%, about 15%, about 20%, about 25%, about 30%, or about 35%. In some embodiments, the triglyceride is present in an amount of at least about 1%, about 5%, about 10%, about 15%, about 20%, about 25%, or about 30%. In some embodiments, the triglyceride is present in an amount of at most about 5% about 10% about 15%, about 20%, about 25%, about 30%, or about 35%. In some embodiments, the oil phase of the lipid vesicle and/or the lipid vesicle portion of the composition comprises a sterol. In some embodiments, the sterol is cholesterol. In some embodiments, the cholesterol may be plant-derived cholesterol. In some embodiments, the plant-derived cholesterol may be PhytoChol®, SyntheChol®, or any other plant-derived cholesterol (e.g., Avanti #700100), or any combination thereof. In some embodiments, the sterol may be phytosterol or a derivative thereof. In some embodiments, the phytosterol or derivative thereof may be phytosterol MM, Advasterol™ 90 IP or 95 IP F, NET Sterol-ISO, canola sterols, sitosterol 700095, lanosterol-95, brassicasterol, or any combination thereof.

In some embodiments, the sterol is present in an amount of about 1% to about 5% (w/w) of the composition. In some embodiments, the sterol is present in an amount of about 1% to about 1.5%, about 1% to about 2%, about 1% to about 2.5%, about 1% to about 3%, about 1% to about 4%, about 1% to about 5%, about 1.5% to about 2%, about 1.5% to about 2.5%, about 1.5% to about 3%, about 1.5% to about 4%, about 1.5% to about 5%, about 2% to about 2.5%, about 2% to about 3%, about 2% to about 4%, about 2% to about 5%, about 2.5% to about 3%, about 2.5% to about 4%, about 2.5% to about 5%, about 3% to about 4%, about 3% to about 5%, or about 4% to about 5% (w/w) of the composition. In some embodiments, the sterol is present in an amount of about 1%, about 1.5%, about 2%, about 2.5%, about 3%, about 4%, or about 5% (w/w) of the composition. In some embodiments, the sterol is present in an amount of at least about 1%, about 1.5%, about 2%, about 2.5%, about 3%, or about 4% (w/w) of the composition. In some embodiments, the sterol is present in an amount of at most about 1.5%, about 2%, about 2.5%, about 3%, about 4%, or about 5% (w/w) of the composition.

### Penetration Enhancers

In some embodiments, the lipid vesicle compositions comprise one or more penetration enhancers. Penetration enhancers act to increase the amount of penetration of the anionic polymer material through one or more layers of skin when applied to the skin of an individual.

In some embodiments, the penetration enhancer is included in the oil-in-water emulsion of the composition. In some embodiments, the penetration enhancer is included in the lipid bilayer of the composition.

There are many types of penetration enhancing agents that may be employed. In some embodiments, the penetration enhancing agent comprising an ionic surfactant, a nonionic surfactant, or a combination thereof.

In some embodiments, the penetration enhancing agent comprises a non-ionic surfactant or a combination of non-ionic surfactants. In some embodiments, the penetration enhancing agent is a single non-ionic surfactant. In some embodiments, the penetration enhancing agent is a combination of at least 2, 3, 4, or more non-ionic surfactants. In some embodiments, the penetration enhancing agent is a combination 2 non-ionic surfactants. In some embodiments, the penetration enhancing agent is a combination 3 non-ionic surfactants.

In some embodiments, the non-ionic surfactant or combination of non-ionic surfactants is selected from polyethylene glycol ethers of fatty alcohols, sorbitan esters, polysorbates, and polyethylene glycol fatty acid esters and combinations thereof.

In some embodiments, the combination of non-ionic surfactants is a combination of a polyethylene glycol ether of a fatty alcohol and a sorbitan ester. In some embodiments, the combination of non-ionic surfactants is a combination of a polyethylene glycol ethers of fatty alcohol and a polysorbate. In some embodiments, the combination of non-ionic surfactants is a combination of a polyethylene glycol ethers of fatty alcohol and a sorbitan ester. In some embodiments, the combination of non-ionic surfactants is a combination of a polyethylene glycol ethers of fatty alcohol and a polyethylene glycol fatty acid ester. In some embodiments, the combination of non-ionic surfactants is a combination of a polyethylene glycol ether of a fatty alcohol, a sorbitan ester, and a polysorbate. In some embodiments, the combination of non-ionic surfactants is a combination of a polyethylene glycol ether of a fatty alcohol, a sorbitan ester, and a polyethylene glycol fatty acid ester. In some embodiments, the combination of non-ionic surfactants is a combination of a polyethylene glycol ether of a fatty alcohol, a polysorbate, and a polyethylene glycol fatty acid ester.

In some embodiments, the combination of non-ionic surfactants comprises a polyethylene glycol fatty acid ester and a sorbitan ester. In some embodiments, the combination of non-ionic surfactants comprises a polyethylene glycol fatty acid ester and a polysorbate. In some embodiments, the combination of non-ionic surfactants is a combination of a polyethylene glycol fatty acid ester, a polysorbate, and a sorbitan ester.

In some embodiments, the non-ionic surfactant comprises a polyethylene glycol (PEG) ether of a fatty alcohol. In some embodiments, the PEG ether of the fatty alcohol comprises from about 2 to about 8 PEG groups and a C₁₂-C₂₂ fatty alcohol. In some embodiments, the polyethylene glycol ether of a fatty alcohol comprises diethylene glycol hexadecyl ether, 2-(2-octadecoxyethoxy)ethanol, diethylene glycol monooleyl ether, polyoxyethylene (2) oleyl ether, polyoxyethylene (3) oleyl ether, or polyoxyethylene (5) oleyl ether, or any combination thereof. In some embodiments, the polyethylene glycol ether of a fatty alcohol comprises 2-(2-octadecoxyethoxy)ethanol. In some embodiments, the PEG ether of a fatty alcohol is super refined Brij® 02 or a derivative thereof.

In some embodiments, the PEG ether of the fatty alcohol is present in an amount of from about 0.5% to about 10% (w/w) of the composition. In some embodiments, the PEG ether of the fatty alcohol is present in an amount of about 0.5% to about 2.5%. In some embodiments, the PEG ether of the fatty alcohol is present in an amount of about 0.5% to about 0.8%, about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the PEG ether of the fatty alcohol is present in an amount of about 0.5%, about 0.8%, about 1%, about 1.2%, about 1.5% about 2%, or about 2.5%. In some embodiments, the PEG ether of the fatty alcohol is present in an amount of at least about 0.5%, about 0.8%, about 1%, about 1.2%, about 1.5%, or about 2%. In some embodiments, the PEG ether of the fatty alcohol is present in an amount of at most about 0.8%, about 1%, about 1.2%, about 1.5%, about 2%, or about 2.5%.

In some embodiments, the non-ionic surfactant comprises a sorbitan ester. In some embodiments, the sorbitan ester is a fatty acid ester. In some embodiments, the sorbitan ester is a C₁₂-C₂₂ fatty acid ester. In some embodiments, the sorbitan ester comprises sorbitan monolaurate, sorbitan monopalmitate, sorbitan monostearate, sorbitan monooleate, sorbitan trioleate, sorbitan sesquioleate, or sorbitan isostearate, or any combinations thereof. In some embodiments, the sorbitan ester comprises sorbitan monolaurate. In some embodiments, the sorbitan ester comprises sorbitan monopalmitate. In some embodiments, the sorbitan ester comprises sorbitan monostearate. In some embodiments, the sorbitan ester comprises sorbitan monooleate. In some embodiments, the sorbitan ester comprises sorbitan trioleate. In some embodiments, the sorbitan ester comprises sorbitan sesquioleate. In some embodiments, the sorbitan ester comprises sorbitan isostearate.

In some embodiments, the sorbitan ester is present in an amount of about 0.5% to about 2.5% (w/w) of the composition. In some embodiments, the sorbitan ester is present in an amount of about 0.5% to about 0.8%, about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the sorbitan ester is present in an amount of about 0.5%, about 0.8%, about 1%, about 1.2%, about 1.5%, about 2%, or about 2.5%. In some embodiments, the sorbitan ester is present in an amount of at least about 0.5%, about 0.8%, about 1%, about 1.2%, about 1.5%, or about 2%. In some embodiments, the sorbitan ester is present in an amount of at most about 0.8%, about 1%, about 1.2%, about 1.5%, about 2%, or about 2.5%.

In some embodiments, the non-ionic surfactant comprises a polysorbate. In some embodiments, the polysorbate comprises polysorbate 20, polysorbate 21, polysorbate 40, polysorbate 60, polysorbate 80, polysorbate 85, or any combination thereof. In some embodiments, the polysorbate is polysorbate 80. In some embodiments, the polysorbate is polysorbate 20.

In some embodiments, the polysorbate is present in an amount of about 0.5% to about 2.5% (w/w) of the composition. In some embodiments, the polysorbate is present in an amount of about 0.5% to about 0.8%, about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the polysorbate is present in an amount of about 0.5%, about 0.8%, about 1%, about 1.2%, about 1.5%, about 2%, or about 2.5%. In some embodiments, the polysorbate is present in an amount of at least about 0.5%, about 0.8%, about 1%, about 1.2% about 1.5%, or about 2%. In some embodiments, the polysorbate is present in an amount of at most about 0.8%, about 1%, about 1.2%, about 1.5%, about 2%, or about 2.5%.

In some embodiments, the non-ionic surfactant comprises a polyethylene glycol (PEG) fatty acid ester. In some embodiments, the PEG fatty acid ester is a PEG chain of about 2-8 subunits comprising C₅-C₂₂ fatty acids affixed to each terminal hydroxyl to form the fatty acid ester. In some embodiments, the PEG fatty acid ester comprises PEG-8 dilaurate, PEG-4 dilaurate, PEG-4 laurate, PEG-8 dioleate, PEG-8 distearate, PEG-8 distearate, PEG-7 glyceryl cocoate, and PEG-20 almond glycerides, or any combination thereof. In some embodiments, the PEG fatty acid ester is PEG-4 dilaurate.

In some embodiments, the PEG fatty acid ester is present in an amount of about 0.5% to about 2.5% (w/w) of the composition. In some embodiments, the PEG fatty acid ester is present in an amount of about 0.5% to about 0.8%, about 0.5% to about 1%, about 0.5% to about 1.2%, about 0.5% to about 1.5%, about 0.5% to about 2%, about 0.5% to about 2.5%, about 0.8% to about 1%, about 0.8% to about 1.2%, about 0.8% to about 1.5%, about 0.8% to about 2%, about 0.8% to about 2.5%, about 1% to about 1.2%, about 1% to about 1.5%, about 1% to about 2%, about 1% to about 2.5%, about 1.2% to about 1.5%, about 1.2% to about 2%, about 1.2% to about 2.5%, about 1.5% to about 2%, about 1.5% to about 2.5%, or about 2% to about 2.5%. In some embodiments, the PEG fatty acid ester is present in an amount of about 0.5%, about 0.8%, about 1%, about 1.2%, about 1.5%, about 2%, or about 2.5%. In some embodiments, the PEG fatty ester is present in an amount of at least about 0.5%, about 0.8%, about 1%, about 1.2%, about 1.5%, or about 2%. In some embodiments, the PEG fatty acid ester is present in an amount of at most about 0.8%, about 1%, about 1.2%, about 1.5%, about 2%, or about 2.5%.

In some embodiments, the non-ionic surfactant has a hydrophobic-lipophilic balance (HLB) of about 10 or less. In some embodiments, the non-ionic surfactant may be Cithrol GMS 40. In some embodiments, the composition comprises a plurality of non-ionic surfactants, each having an HLB of about 10 or less. In some embodiments, the non-ionic surfactant with an HLB of 10 or less is selected from the Table 1, or any combination thereof.

In some embodiments, the non-ionic surfactant or combination of non-ionic surfactants are present in an amount of about 0.5% to about 10% (w/w) of the composition. In some embodiments, the non-ionic surfactant or combination of non-ionic surfactants are present in an amount of about 0.5% to about 1%, about 0.5% to about 1.5%, about 0.5% to about 2%, about 0.5% to about 3%, about 0.5% to about 4%, about 0.5% to about 5%, about 0.5% to about 6%, about 0.5% to about 7%, about 0.5% to about 8%, about 0.5% to about 10%, about 1% to about 1.5%, about 1% to about 2%, about 1% to about 3%, about 1% to about 4%, about 1% to about 5%, about 1% to about 6%, about 1% to about 7%, about 1% to about 8%, about 1% to about 10%, about 1.5% to about 2%, about 1.5% to about 3%, about 1.5% to about 4%, about 1.5% to about 5%, about 1.5% to about 6%, about 1.5% to about 7%, about 1.5% to about 8%, about 1.5% to about 10%, about 2% to about 3%, about 2% to about 4%, about 2% to about 5%, about 2% to about 6%, about 2% to about 7%, about 2% to about 8%, about 2% to about 10%, about 3% to about 4%, about 3% to about 5%, about 3% to about 6%, about 3% to about 7%, about 3% to about 8%, about 3% to about 10% about 4% to about 5%, about 4% to about 6%, about 4% to about 7%, about 4% to about 8% about 4% to about 10%, about 5% to about 6%, about 5% to about 7%, about 5% to about 8%, about 5% to about 10%, about 6% to about 7%, about 6% to about 8%, about 6% to about 10%, about 7% to about 8%, about 7% to about 10%, or about 8% to about 10%. In some embodiments, the non-ionic surfactant or combination of non-ionic surfactants are present in an amount of about 0.5%, about 1%, about 1.5% about 2%, about 3%, about 4%, about 5%, about 6%, about 7%, about 8%, or about 10%. In some embodiments, the non-ionic surfactant or combination of non-ionic surfactants are present in an amount of at least about 0.5%, about 1% about 1.5%, about 2%, about 3%, about 4%, about 5%, about 6%, about 7%, or about 8%. In some embodiments, the non-ionic surfactant or combination of non-ionic surfactants are present in an amount of at most about 1%, about 1.5%, about 2%, about 3%, about 4%, about 5%, about 6%, about 7%, about 8%, or about 10%.

In some embodiments, the composition comprises a non-ionic surfactant in the oil-in-water emulsion, the lipid bilayer, or both. In some embodiments, the composition comprises a non-ionic surfactant in the oil-in-water emulsion. In some embodiments, the composition comprises a non-ionic surfactant in the lipid bilayer. In some embodiments, the composition comprises a non-ionic surfactant in the oil-in-water emulsion and the lipid bilayer, wherein the composition comprises two or more different non-ionic surfactants.

In some embodiments, the penetration enhancing agent comprises a salicylate ester or a nicotinate ester. In some embodiments, the ester is a C₁-C₆ alkyl ester or a benzyl ester. In some embodiments, the penetration enhancing agent comprises methyl salicylate or benzyl nicotinate. In some embodiments, the penetration enhancing agent is a nicotinate ester present in an amount of up to about 0.1%, 0.5%, 1%, 2%, or 3% (w/w) of the composition. In some embodiments, the nicotinate ester is present in an amount of from about 0.1% to about 3%, about 0.1% to about 2%, or about 0.10% to about 10%.

In some embodiments, the penetration enhancing agent comprises a fatty acid acylated amino acid. In some embodiments, the fatty acid acylated amino acid is lysine. In some embodiments, the lysine is mono-acylated with a fatty acid. In some embodiments, the penetration enhancing agent is monoloauryl lysine. In some embodiments, the lysine is di-acylated. In some embodiments, the penetration enhancing agent is dipalmitoyllysine. In some embodiments, the fatty acylated amino acid is present in an amount of up to about 1%, up to about 2%, up to about 3%, up to about 4%, or up to about 5% (w/w) of the composition. In some embodiments, the fatty acylated amino acid is present in an amount of from about 0.1% to about 5%, from about 0.10% to about 4%, from about 0.10% to about 3%, from about 0.10% to about 2%, from about 0.5% to about 5%, from about 0.5% to about 4%, from about 0.5% to about 3%, from about 0.5% to about 2%, from about 1% to about 5%, from about 1% to about 4%, from about 1% to about 3%, from about 1% to about 2%, or from about 1.5% to about 2.5%.

### Cationic Surfactants

In some embodiments, the composition further comprises a cationic surfactant. In some embodiments, the cationic surfactant is used to stabilize the water-in-oil emulsion (e.g., at the submicron emulsion stage prior to lipid vesicle formation). In some embodiments, the cationic surfactant is a mono-cationic surfactant. In some embodiments, the mono-cationic surfactant is net-mono-cationic (e.g., a phosphate salt comprising two side chains each with a single cationic functionality, which is partially neutralized by a phosphate anion).

In some embodiments, the mono-cationic surfactant is a fatty-amide derived propylene glycol-diammonium phosphate ester. In some embodiments, the mono-cationic surfactant is linoleamidopropyl PG-dimonium chloride phosphate.

In some embodiments, the fatty amide derived propylene glycol-diammonium phosphate ester is present in an amount of about 1% to about 10% (w/w) of the composition. In some embodiments, the fatty amide derived propylene glycol-diammonium phosphate ester is present in an amount of about 1% to about 2%, about 1% to about 3%, about 1% to about 4%, about 1% to about 5%, about 1% to about 6%, about 1% to about 7%, about 1% to about 8% about 1% to about 9%, about 1% to about 10%, about 2% to about 3%, about 2% to about 4%, about 2% to about 5%, about 2% to about 6%, about 2% to about 7%, about 2% to about 8%, about 2% to about 9%, about 2% to about 10%, about 3% to about 4%, about 3% to about 5%, about 3% to about 6%, about 3% to about 7%, about 3% to about 8%, about 3% to about 9%, about 3% to about 10%, about 4% to about 5%, about 4% to about 6%, about 4% to about 7%, about 4% to about 8%, about 4% to about 9%, about 4% to about 10% about 5% to about 6%, about 5% to about 7%, about 5% to about 8%, about 5% to about 9%, about 5% to about 10%, about 6% to about 7%, about 6% to about 8%, about 6% to about 9%, about 6% to about 10%, about 7% to about 8%, about 7% to about 9%, about 7% to about 10%, about 8% to about 9%, about 8% to about 10%, or about 9% to about 10%. In some embodiments, the fatty amide derived propylene glycol-diammonium phosphate ester is present in an amount of about 1%, about 2%, about 3%, about 4%, about 5%, about 6%, about 7%, about 8%, about 9%, or about 10% (w/w) of the composition. In some embodiments, the fatty amide derived propylene glycol-diammonium phosphate ester is present in an amount of at least about 1% about 2%, about 3%, about 4%, about 5%, about 6%, about 7%, about 8%, or about 9%. In some embodiments, the fatty amide derived propylene glycol-diammonium phosphate ester is present in an amount of at most about 2%, about 3%, about 4%, about 5% about 6%, about 7%, about 8%, about 9%, or about 10%.

### Additional Components

In some embodiments, the vesicle composition comprises additional components. In some embodiments, these additional components improve one or more properties of the vesicles without dramatically altering the delivery of the anionic polymer material.

In some embodiments, the vesicle composition further comprises one or more viscosity enhancing agents. In some embodiments, the viscosity enhancing agents thicken the composition for increased stability and/or feel to a user of the vesicle composition. In some embodiments, the viscosity enhancing agents also act as surfactants. In some embodiments, the viscosity enhancing agent comprises one or more of a fatty alcohol, a wax, a fatty ester of glycerol, or any combination thereof. In some embodiments, the fatty alcohol is a C₅-C₂₀ fatty alcohol. In some embodiments, the fatty alcohol is cetyl alcohol. In some embodiments, the cetyl alcohol is Crodacol C95. In some embodiments, the wax is a naturally occurring or synthetic wax. In some embodiments, the wax is beeswax. In some embodiment, the wax is synthetic beeswax. In some embodiments, the synthetic beeswax is Syncrowax™ BB4. In some embodiments, the synthetic beeswax is non-animal derived beeswax. In some embodiments the non-animal derived beeswax is Syncrowax™ SB1. In some embodiments, the fatty ester of glycerol is a monoester. In some embodiments, the monoester is an ester of a C₈-C₂₄ fatty acid. In some embodiments, the fatty ester of glycerol is glycerol monostearate.

In some embodiments, the viscosity enhancing agents are present in an amount of from about 0.5% to about 10% (w/w) of the composition. In some embodiments, the viscosity enhancing agents are present in an amount of from about 0.5% to about 5%, about 0.5% to about 5%, about 0.5% to about 4%, about 0.5% to about 3%, or from about 0.5% to about 2% (w/w) of the composition. In some embodiments, the viscosity enhancing agents comprise a fatty alcohol in an amount of up to about 2%, a wax in an amount of up to about 2%, and a fatty ester of glycerol in an amount of up to about 5%. In some embodiments, the fatty alcohol is present in an amount of from about 0.1 to about 1.5%. In some embodiments, the wax is present in an amount of from about 0.1% to about 1%. In some embodiments, the fatty ester of glycerol is present in an amount of from about 0.5% to about 2%.

In some embodiments, the vesicle composition further comprises one or more of a thickener, a preservative, a moisturizer, an emollient, a humectant, or any combination thereof. In some embodiments, the vesicle composition further comprises a thickener. In some embodiments, the vesicle composition further comprises a preservative. In some embodiments, the vesicle composition further comprises a moisturizer (e.g., isopropyl myristate). In some embodiments, the vesicle composition further comprises an emollient. In some embodiments, the vesicle composition further comprises a humectant.

In some embodiments, the vesicle composition further comprises a humectant. In some embodiments, the composition comprises glycerol. In some embodiments, the glycerol is present in an amount of from about 0.5% to about 25%, about 0.5% to about 20%, about 0.5% to about 15%, or about 0.5% to about 10% (w/w) of the composition. In some embodiments, the glycerol is present in an amount of about 1% to about 10%. In some embodiments, the glycerol is present in an amount of about 1% to about 2%, about 1% to about 4%, about 1% to about 6%, about 1% to about 8%, about 1% to about 10%, about 2% to about 4%, about 2% to about 6%, about 2% to about 8%, about 2% to about 10%, about 4% to about 6%, about 4% to about 8%, about 4% to about 10%, about 6% to about 8%, about 6% to about 10%, or about 8% to about 10%. In some embodiments, the glycerol is present in an amount of about 1%, about 2%, about 4%, about 6%, about 8%, or about 10%. In some embodiments, the glycerol is present in an amount of at least about 1%, about 2%, about 4%, about 6%, or about 8%. In some embodiments, the glycerol is present in an amount of at most about 2%, about 4%, about 6%, about 8%, or about 10%.

In some embodiments, the vesicle composition further comprises a preservative. In some embodiments, the preservative is a paraben ester. In some embodiments, the preservative is methylparaben or propylparaben, or a combination thereof. In some embodiments, the preservative is present in an amount of up to about 1%, up to about 0.9%, up to about 0.8%, up to about 0.7%, up to about 0.6%, up to about 0.5%, up to about 0.4%, up to about 0.3%, up to about 0.2% (w/w) of the composition.

In some embodiments, the additional components comprise purified water. In some embodiments, purified water is present in an amount of about 50% to 90% (w/w). In some embodiments, purified water is present in an amount of about 50% to about 55%, about 50% to about 60%, about 50% to about 65%, about 50% to about 70%, about 50% to about 75%, about 50% to about 80%, about 50% to about 85%, about 50% to about 90%, about 55% to about 60%, about 55% to about 65%, about 55% to about 70%, about 55% to about 75%, about 55% to about 80%, about 55% to about 85%, about 55% to about 90%, about 60% to about 65%, about 60% to about 70%, about 60% to about 75%, about 60% to about 80%, about 60% to about 85%, about 60% to about 90%, about 65% to about 70%, about 65% to about 75%, about 65% to about 80%, about 65% to about 85%, about 65% to about 90%, about 70% to about 75%, about 70% to about 80%, about 70% to about 85%, about 70% to about 90%, about 75% to about 80%, about 75% to about 85%, about 75% to about 90%, about 80% to about 85%, about 80% to about 90%, or about 85% to about 90%. In some embodiments, purified water is present in an amount of about 50%, about 55%, about 60%, about 65%, about 70%, about 75%, about 80%, about 85%, or about 90%. In some embodiments, purified water is present in an amount of at least about 50%, about 55%, about 60%, about 65%, about 70%, about 75%, about 80%, or about 85%. In some embodiments, purified water is present in an amount of at most about 55%, about 60%, about 65%, about 70%, about 75%, about 80%, about 85%, or about 90%.

### Exemplary Compositions for Delivery of Peptide Antagonists

Provided below are exemplary compositions for the delivery of peptide antagonists. The embodiments below may additional comprise any of the other ingredients or components provided herein.

Peptide Composition 1: In one aspect, provided herein, is a lipid vesicle composition comprising
(a) lipid vesicles each comprising a lipid bilayer comprising vesicle forming lipids, wherein the vesicle forming lipids are present in an amount of from about 5% to about 20%;
(b) an oil-in-water emulsion entrapped in the lipid vesicles, stabilized by one or more surfactants;
(c) a peptide antagonist of muscle-type nicotinic acetylcholine receptors in an amount of from about 0.1 mg/mL to about 50 mg/mL entrapped in the lipid bilayer and/or the oil-in-water emulsion,
wherein the composition further comprises:
   a fatty amide derived propylene glycol-diammonium phosphate ester in an amount of from about 1% to about 10%; and
   a non-ionic surfactant in an amount of from about 0.1% to about 3%.

In some embodiments, the oil component is present in an amount of from about 2.5% to about 20%.

In some embodiments, the lipid vesicle composition comprises the peptide antagonist in an amount of about 0.1 mg/mL to about 0.5 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL. In some embodiments the lipid vesicle composition comprises the peptides antagonist in an amount of about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the lipid vesicle composition comprises the peptides antagonist in an amount of about 1 mg/mL, about 2 mg/mL, about 3 mg/mL, about 4 mg/mL, or about 5 mg/mL.

In some embodiments, the composition further comprises a fatty acylated amino acid in an amount of from about 0.5% to about 3%. In some embodiments, the fatty acylated amino acid is monoloauryl lysine.

In some embodiments, the lipid vesicle composition further comprises viscosity enhancing agents in an amount of from about 0.5% to about 5%. In some embodiments, the viscosity enhancing agents comprise one or more of a fatty alcohol, a wax, a fatty ester of glycerol, or any combination thereof.

In some embodiments, the non-ionic surfactant comprises a PEG ether of a fatty alcohol.

In some embodiments, the lipid vesicle composition further comprises an anionic polymer material in an amount of from about 0.01 mg/mL to about 10 mg/mL entrapped in the lipid bilayer, the oil-in-water emulsion, or a combination thereof. In some embodiments, the lipid vesicle composition comprises the anionic polymer material in an amount of about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 1.25 mg/mL, about 0.01 mg/mL to about 1.5 mg/mL, about 0.01 mg/mL to about 1.75 mg/mL, about 0.01 mg/mL to about 2 mg/mL, about 0.01 mg/mL to about 5 mg/mL, about 0.01 mg/mL to about 10 mg/mL, about 0.1 mg/mL to about 0.5 mg/mL, about 0.1 mg/mL to about 1 mg/mL, about 0.1 mg/mL to about 1.25 mg/mL, about 0.1 mg/mL to about 1.5 mg/mL, about 0.1 mg/mL to about 1.75 mg/mL, about 0.1 mg/mL to about 2 mg/mL, about 0.1 mg/mL to about 5 mg/mL, about 0.1 mg/mL to about 10 mg/mL, about 0.5 mg/mL to about 1 mg/mL, about 1 mg/mL to about 1.5 mg/mL, about 1 mg/mL to about 1.75 mg/mL, about 1 mg/mL to about 2 mg/mL, about 1 mg/mL to about 5 mg/mL, about 1 mg/mL to about 10 mg/mL. In some embodiments, the lipid vesicle composition comprises the anionic polymer material in an amount of about 0.01 mg/mL, about 0.05 mg/mL, about 0.1 mg/mL, about 0.5 mg/mL, about 1 mg/mL, about 1.25 mg/mL, about 1.5 mg/mL, about 1.75 mg/mL, about 2 mg/mL, about 5 mg/mL, or about 10 mg/mL.

Peptide Composition 2: In one aspect, provided herein, is a lipid vesicle composition comprising
(a) lipid vesicles each comprising a lipid bilayer comprising vesicle forming lipids, wherein the vesicle forming lipids are present in an amount of from about 2% to about 20%;
(b) an oil-in-water emulsion entrapped in the lipid vesicles, and stabilized by one or more surfactants;
(c) a peptide antagonist of muscle-type nicotinic acetylcholine receptors in an amount of from about 0.1 mg/mL to about 50 mg/mL entrapped in the lipid bilayer and/or the oil-in-water emulsion,
wherein the composition further comprises:
   a PEG fatty acid ester in an amount of from about 0.1% to about 2%;
   a polysorbate in an amount of from about 0.5% to about 3%; and
   a sorbate ester in an amount of from about 0.1% to about 2%.

In some embodiments, the oil component is present in an amount of from about 2.5% to about 20%.

In some embodiments, the lipid vesicle composition comprises the peptide antagonist in an amount of about 0.1 mg/mL to about 0.5 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL. In some embodiments the lipid vesicle composition comprises the peptides antagonist in an amount of about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the lipid vesicle composition comprises the peptides antagonist in an amount of about 1 mg/mL, about 2 mg/mL, about 3 mg/mL, about 4 mg/mL, or about 5 mg/mL.

In some embodiments, the lipid vesicle composition further comprises viscosity enhancing agents in an amount of from about 0.5% to about 5%. In some embodiments, the viscosity enhancing agents comprise one or more of a fatty alcohol, a wax, a fatty ester of glycerol, or any combination thereof.

In some embodiments, the PEG fatty acid ester comprises PEG4-dilaurate. In some embodiments, the polysorbate is polysorbate 80. In some embodiments, the sorbate ester is sorbitan palmitate.

In some embodiments, the lipid vesicle composition further comprises an anionic polymer material in an amount of from about 0.01 mg/mL to about 10 mg/mL entrapped in the lipid bilayer, the oil-in-water emulsion, or a combination thereof. In some embodiments, the lipid vesicle composition comprises the anionic polymer material in an amount of about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 1.25 mg/mL, about 0.01 mg/mL to about 1.5 mg/mL, about 0.01 mg/mL to about 1.75 mg/mL, about 0.01 mg/mL to about 2 mg/mL, about 0.01 mg/mL to about 5 mg/mL, about 0.01 mg/mL to about 10 mg/mL, about 0.1 mg/mL to about 0.5 mg/mL, about 0.1 mg/mL to about 1 mg/mL, about 0.1 mg/mL to about 1.25 mg/mL, about 0.1 mg/mL to about 1.5 mg/mL, about 0.1 mg/mL to about 1.75 mg/mL, about 0.1 mg/mL to about 2 mg/mL, about 0.1 mg/mL to about 5 mg/mL, about 0.1 mg/mL to about 10 mg/mL, about 0.5 mg/mL to about 1 mg/mL, about 1 mg/mL to about 1.5 mg/mL, about 1 mg/mL to about 1.75 mg/mL, about 1 mg/mL to about 2 mg/mL, about 1 mg/mL to about 5 mg/mL, about 1 mg/mL to about 10 mg/mL. In some embodiments, the lipid vesicle composition comprises the anionic polymer material in an amount of about 0.01 mg/mL, about 0.05 mg/mL, about 0.1 mg/mL, about 0.5 mg/mL, about 1 mg/mL, about 1.25 mg/mL, about 1.5 mg/mL, about 1.75 mg/mL, about 2 mg/mL, about 5 mg/mL, or about 10 mg/mL.

Peptide Composition 3: In one aspect, provided herein, is a lipid vesicle composition comprising
(a) lipid vesicles each comprising a lipid bilayer comprising vesicle forming lipids, wherein the vesicle forming lipids are present in an amount of from about 5% to about 20%;
(b) an oil-in-water emulsion entrapped in the lipid vesicles, and stabilized by one or more surfactants;
(c) a peptide antagonist of muscle-type nicotinic acetylcholine receptors in an amount of from about 0.1 mg/mL to about 50 mg/mL entrapped in the lipid bilayer and/or the oil-in-water emulsion,
wherein the composition further comprises: and
   a fatty amide derived propylene glycol-diammonium phosphate ester in an amount of from about 1% to about 10%;
   a PEG ether of a fatty alcohol in an amount of from about 0.1% to about 3%;
   a polysorbate in an amount of from about 0.5% to about 3%; and
   a sorbate ester in an amount of from about 0.1% to about 2%.

In some embodiments, the oil component is present in an amount of from about 2.5% to about 20%.

In some embodiments, the lipid vesicle composition comprises the peptide antagonist in an amount of about 0.1 mg/mL to about 0.5 mg/mL, about 0.1 mg/mL to about 1 mg/mL, about 0.1 mg/mL to about 2 mg/mL, about 0.1 mg/mL to about 3 mg/mL, about 0.1 mg/mL to about 4 mg/mL, about 0.1 mg/mL to about 5 mg/mL, about 0.1 mg/mL to about 10 mg/mL, about 0.1 mg/mL to about 20 mg/mL, about 0.1 mg/mL to about 50 mg/mL. In some embodiments the lipid vesicle composition comprises the peptides antagonist in an amount of about 0.1 mg/mL, about 0.5 mg/mL, about 1 mg/mL, about 2 mg/mL, about 3 mg/mL, about 4 mg/mL, about 5 mg/mL, about 10 mg/mL, about 20 mg/mL, or about 50 mg/mL. In some embodiments, the lipid vesicle composition comprises the peptides antagonist in an amount of about 1 mg/mL, about 2 mg/mL, about 3 mg/mL, about 4 mg/mL, or about 5 mg/mL.

In some embodiments, the lipid vesicle composition further comprises viscosity enhancing agents in an amount of from about 0.5% to about 5%. In some embodiments, the viscosity enhancing agents comprise one or more of a fatty alcohol, a wax, a fatty ester of glycerol, or any combination thereof.

In some embodiments, the polysorbate is polysorbate 80. In some embodiments, the sorbate ester is sorbitan palmitate. In some embodiments, the PEG ether of the fatty alcohol is diethylene glycol monooleyl ether.

In some embodiments, the lipid vesicle composition further comprises an anionic polymer material in an amount of from about 0.01 mg/mL to about 10 mg/mL entrapped in the lipid bilayer, the oil-in-water emulsion, or a combination thereof. In some embodiments, the lipid vesicle composition comprises the anionic polymer material in an amount of about 0.01 mg/mL to about 0.05 mg/mL, about 0.01 mg/mL to about 0.1 mg/mL, about 0.01 mg/mL to about 0.5 mg/mL, about 0.01 mg/mL to about 1 mg/mL, about 0.01 mg/mL to about 1.25 mg/mL, about 0.01 mg/mL to about 1.5 mg/mL, about 0.01 mg/mL to about 1.75 mg/mL, about 0.01 mg/mL to about 2 mg/mL, about 0.01 mg/mL to about 5 mg/mL, about 0.01 mg/mL to about 10 mg/mL, about 0.1 mg/mL to about 0.5 mg/mL, about 0.1 mg/mL to about 1 mg/mL, about 0.1 mg/mL to about 1.25 mg/mL, about 0.1 mg/mL to about 1.5 mg/mL, about 0.1 mg/mL to about 1.75 mg/mL, about 0.1 mg/mL to about 2 mg/mL, about 0.1 mg/mL to about 5 mg/mL, about 0.1 mg/mL to about 10 mg/mL, about 0.5 mg/mL to about 1 mg/mL, about 1 mg/mL to about 1.5 mg/mL, about 1 mg/mL to about 1.75 mg/mL, about 1 mg/mL to about 2 mg/mL, about 1 mg/mL to about 5 mg/mL, about 1 mg/mL to about 10 mg/mL. In some embodiments, the lipid vesicle composition comprises the anionic polymer material in an amount of about 0.01 mg/mL, about 0.05 mg/mL, about 0.1 mg/mL, about 0.5 mg/mL, about 1 mg/mL, about 1.25 mg/mL, about 1.5 mg/mL, about 1.75 mg/mL, about 2 mg/mL, about 5 mg/mL, or about 10 mg/mL.

### Methods of Use of Lipid Vesicle Compositions Provided Herein

The lipid vesicle compositions provided herein are contemplated for cosmetic uses in a subject, for indications including but not limited to the prevention or temporary improvement of the appearance of one or more of: skin wrinkles; skin laxity; moderate to severe glabellar lines associated with corrugator and/or procerus muscle activity; moderate to severe lateral canthal lines associated with orbicularis oculi activity (crow's feet lines); and moderate to severe forehead lines associated with frontalis muscle activity.

In certain embodiments, including pharmaceutical embodiments, the lipid vesicle compositions provided herein are contemplated for pharmaceutical use in a subject, for indications including but not limited to: prevention or temporary improvement of the appearance of one or more of skin wrinkles, e.g., in the face, skin laxity, moderate to severe glabellar lines associated with corrugator and/or procerus muscle activity, moderate to severe lateral canthal lines associated with orbicularis oculi activity (crow's feet lines), and moderate to severe forehead lines associated with frontalis muscle activity.

In some embodiments, the subject is a mammal. In specific embodiments, the mammal is a human. In some embodiments, the human subject is a pediatric or adult subject, of any age.

### Methods for Using Cosmetic or Pharmaceutical Compositions

In certain embodiments, including pharmaceutical embodiments, the present disclosure also relates to methods for using cosmetic or pharmaceutical compositions comprising a peptide antagonist or an anionic polymer material such as hyaluronic acid. In some embodiments, the disclosure relates to methods for using the cosmetic or pharmaceutical composition to prevent or temporarily improve the appearance in a subject of one or more of skin wrinkles, e.g., in the face, skin laxity, moderate to severe glabellar lines associated with corrugator and/or procerus muscle activity, moderate to severe lateral canthal lines associated with orbicularis oculi activity (crow's feet lines), and moderate to severe forehead lines associated with frontalis muscle activity, comprising applying an effective amount of the cosmetic or pharmaceutical composition to the skin of the subject. In some embodiments, the disclosure relates to methods for using the cosmetic or pharmaceutical composition to improve the appearance of the lips of a subject, e.g., by making the lips appear fuller. In some embodiments, the cosmetic or pharmaceutical composition is used for enhancing lip fullness, lip volume, lip smoothness, lip color, or a combination thereof. In some embodiments, the cosmetic or pharmaceutical composition provides fuller and/or natural-looking lips for a subject. In some embodiments, the cosmetic or pharmaceutical composition is used to restore any one of volume, definition, suppleness, or fullness to the lips of the subject. In some embodiments, the cosmetic or pharmaceutical composition is used to diminish or visible remove lines or wrinkles on the lips of the subject. In some embodiments, the cosmetic or pharmaceutical composition creates enhanced color in the lips (e.g., rosy flush). In some embodiments, the cosmetic or pharmaceutical composition provides one or more of volume, suppleness, and definition to the lips of the subject.

In some embodiments, the lipid vesicle composition is topically applied to a subject. Topical application as referred to herein can refer to application onto one or more surface, e.g., keratinous tissue. In some embodiments, the topical composition is administered to the skin of a subject. In some embodiments, the skin is the facial skin of the subject. In some embodiments, the skin comprises the lips of the subject. Topical application may relate to direct application to the desired area. In certain embodiments, including pharmaceutical embodiments, a topical cosmetic or pharmaceutical composition or preparation can be applied by, e.g., pouring, dropping, or spraying, when present as a liquid or aerosol composition; smoothing, rubbing, spreading, and the like, when in ointment, lotion, cream, gel, or a like composition; dusting, when a powder; or by any other appropriate means.

In some embodiments, the lipid vesicle composition is formulated in a form suitable for topical application. In some embodiments, the lipid vesicle composition is formulated as a cream, a lotion, a suspension, or an emulsion. In some embodiments, the lipid vesicle composition is formulated as a cream. In some embodiments, the lipid vesicle composition is formulated as a lotion. In some embodiments, the lipid vesicle composition is formulated as a suspension.

In some embodiments, the subject uses or is treated with a topical application comprising an effective amount of the lipid vesicle composition one time or more during a course of usage or treatment, e.g., 1-3 times per day, 1-21 times per week, 1 time per day, 2 times per day, or 3 times per day. In some embodiments, a subject uses or is treated with an effective amount of the lipid vesicle composition about 1 time per week to about 12 times per week. In some embodiments, a subject uses or is treated with an effective amount of the lipid vesicle composition at least about 1 time per week. In some embodiments, a subject uses or is treated with an effective amount of the lipid vesicle composition at most about 12 times per week. In some embodiments, a subject uses or is treated with an effective amount of the lipid vesicle composition about 1 time per week to about 2 times per week, about 1 time per week to about 3 times per week, about 1 time per week to about 4 times per week, about 1 time per week to about 5 times per week, about 1 time per week to about 6 times per week, about 1 time per week to about 7 times per week, about 1 time per week to about 8 times per week, about 1 time per week to about 9 times per week, about 1 time per week to about 10 times per week, about 1 time per week to about 11 times per week, about 1 time per week to about 12 times per week, about 2 times per week to about 3 times per week, about 2 times per week to about 4 times per week, about 2 times per week to about 5 times per week, about 2 times per week to about 6 times per week, about 2 times per week to about 7 times per week, about 2 times per week to about 8 times per week, about 2 times per week to about 9 times per week, about 2 times per week to about 10 times per week, about 2 times per week to about 11 times per week, about 2 times per week to about 12 times per week, about 3 times per week to about 4 times per week, about 3 times per week to about 5 times per week, about 3 times per week to about 6 times per week, about 3 times per week to about 7 times per week, about 3 times per week to about 8 times per week, about 3 times per week to about 9 times per week, about 3 times per week to about 10 times per week, about 3 times per week to about 11 times per week, about 3 times per week to about 12 times per week, about 4 times per week to about 5 times per week, about 4 times per week to about 6 times per week, about 4 times per week to about 7 times per week, about 4 times per week to about 8 times per week, about 4 times per week to about 9 times per week, about 4 times per week to about 10 times per week, about 4 times per week to about 11 times per week, about 4 times per week to about 12 times per week, about 5 times per week to about 6 times per week, about 5 times per week to about 7 times per week, about 5 times per week to about 8 times per week, about 5 times per week to about 9 times per week, about 5 times per week to about 10 times per week, about 5 times per week to about 11 times per week, about 5 times per week to about 12 times per week, about 6 times per week to about 7 times per week, about 6 times per week to about 8 times per week, about 6 times per week to about 9 times per week, about 6 times per week to about 10 times per week, about 6 times per week to about 11 times per week, about 6 times per week to about 12 times per week, about 7 times per week to about 8 times per week, about 7 times per week to about 9 times per week, about 7 times per week to about 10 times per week, about 7 times per week to about 11 times per week, about 7 times per week to about 12 times per week, about 8 times per week to about 9 times per week, about 8 times per week to about 10 times per week, about 8 times per week to about 11 times per week, about 8 times per week to about 12 times per week, about 9 times per week to about 10 times per week, about 9 times per week to about 11 times per week, about 9 times per week to about 12 times per week, about 10 times per week to about 11 times per week, about 10 times per week to about 12 times per week, or about 11 times per week to about 12 times per week. In some embodiments, a subject uses or is treated with an effective amount of the lipid vesicle composition about 1 time per week, about 2 times per week, about 3 times per week, about 4 times per week, about 5 times per week, about 6 times per week, about 7 times per week, about 8 times per week, about 9 times per week, about 10 times per week, about 11 times per week, about 12 times per week, about 13 times per week, or about 14 times per week.

In some embodiments, one or more layers of a lipid vesicle composition of the disclosure is applied to the skin of the subject at a given time. In some embodiments, a subsequent layer may be applied after a previous layer of the lipid vesicle composition is fully absorbed into the skin of the subject. In some embodiments, the lipid vesicle composition may take a couple of seconds (e.g., one second, two seconds, three second, five seconds, ten seconds, fifteen seconds, thirty seconds, etc.) to fully absorb into the skin of the subject. In some embodiments, one, two, three, four, five, six, or seven layers of the lipid vesicle composition is applied to the skin of the subject at a given time. In some embodiments, the lipid vesicle composition is applied to the skin of the subject one or more times a day (e.g., 1-3 times per day, 1 time per day, 2 times per day, 3 times per day, etc.). In some embodiments, the lipid vesicle composition is applied to the skin of the subject one or more times a week (e.g., 1-21 times per week, 1-14 times per week, 1-7 times per week, etc.). In some embodiments, the lipid vesicle composition is applied to the skin of the subject daily. In some embodiments, one or more layers of the lipid vesicle composition is applied to the skin of the subject once a day for one or more days. In some embodiments, two or more layers of the lipid vesicle composition is applied to the skin of the subject once a day for one or more days. In some embodiments, three or more layers of the lipid vesicle composition is applied to the skin of the subject once a day for one or more days. In some embodiments, one or more layers of the lipid vesicle composition is applied to the skin of the subject twice a day for one or more days. In some embodiments, two or more layers of the lipid vesicle composition is applied to the skin of the subject twice a day for one or more days. In some embodiments, three or more layers of the lipid vesicle composition is applied to the skin of the subject twice a day for one or more days. In some embodiments, the lipid vesicle composition is applied to the skin of the subject for at least one day, two days, three days, four days, five days, six days, one week, two weeks, three weeks, one month, two months, three months, six months, one year. In some embodiments, the lipid vesicle composition is applied to the skin of the subject for more than one day, two days, three days, four days, five days, six days, one week, two weeks, three weeks, one month, two months, three months, six months, nine months, or one year. In some embodiments, one or more layers of the lipid vesicle composition is applied to the skin of the subject twice a day for several days, and thereafter is applied three times a day. In some embodiments, five layers of the lipid vesicle composition is applied to the skin of the subject twice a day for five days (e.g., morning and night), and thereafter one to three layers of the lipid vesicle composition is applied to the skin of the subject three times a day (e.g., morning, noon and night).

In some embodiments, a lipid vesicle composition of the disclosure is administered to a subject, for indications including but not limited to: prevention or temporary improvement of the appearance of one or more of skin wrinkles, e.g., in the face, skin laxity, moderate to severe glabellar lines associated with corrugator and/or procerus muscle activity, moderate to severe lateral canthal lines associated with orbicularis oculi activity (crow's feet lines), and moderate to severe forehead lines associated with frontalis muscle activity.

In some embodiments, a lipid vesicle composition of the disclosure is administered to a subject, for indications including but not limited to: temporary improvement of the appearance of lip fullness. In some embodiments, a lipid vesicle composition of the disclosure is used with other products, including, but not limited to Vaseline, lip balms, lipstick, lip tints, lip gloss, lip moisturizers, lip conditioners, sunscreen, etc.

In some embodiments, a topical cosmetic composition of the disclosure is self-applied or administered by a subject. In certain embodiments, including pharmaceutical embodiments, a cosmetic or pharmaceutical composition of the disclosure is applied or administered by a medical professional, e.g., in a medical office setting.

### Methods of Making Lipid Vesicle Compositions Provided Herein

Also provided herein are method of making lipid vesicle compositions. In some embodiments, compositions of the disclosure as described above are prepared by mixing oil components of the oil-in-water emulsion with aqueous components of the oil-in-water emulsion wherein either the oil components or aqueous components of the oil-in-water emulsion comprises one or more surfactants for emulsification of the oil component with the aqueous component of the oil-in-water emulsion. In an embodiment, the surfactant is mixed with the aqueous component and added to the oil for formation of an emulsion. The oil-in-water emulsion is then mixed with the solubilized vesicle-forming lipid and, if added, other lipid components under mixing conditions effective to form the lipid vesicles (e.g., multisomes).

In some embodiments, one or more penetration enhancing agents and the one or more compounds (e.g., anionic polymer material, one or more peptides, etc.) added to oil component of the oil-in-water emulsion, to the aqueous component of the oil-in-water emulsion or both. Alternatively, or in addition to, the one or more penetration enhancing agents and/or the one or more compounds can be added to the lipid component.

In one aspect, provided herein, is a method of preparing a lipid vesicle composition provided herein, comprising: a) preparing an oil-in-water emulsion comprising an active ingredient, by mixing oil components of the oil-in-water emulsion with aqueous components of the oil-in-water emulsion; b) solubilizing vesicle forming lipids in an acceptable solvent other than water; c) adding the oil-in-water emulsion to the solubilized vesicle forming lipids; and d) mixing the oil-in-water emulsion and the solubilized vesicle forming lipids under mixing conditions effective to form the lipid vesicles comprising a lipid bilayer comprising vesicle forming lipids, and an oil-in-water emulsion entrapped in the lipid vesicles. In some embodiments, the active ingredient is a peptide provided herein. In some embodiments, the active ingredient is an anionic polymer material provided herein.

In some embodiments, the method further comprising adding one or more of the additional components provided herein (e.g., penetration enhancing agents, viscosity enhancing agents, etc.)

In some embodiments, mixing oil components of the oil-in-water emulsion with aqueous components of the oil-in-water emulsion vesicles of step a) and/or the mixing conditions of step e) comprises using agitation such as homogenization or emulsification, or micro-emulsion techniques which do not involve agitation. In an embodiment, the mixing comprises high pressure homogenizing. The high pressure homogenizing provides relatively precise control over the composition of the lipid vesicles. High pressure homogenizing is suitable for small molecules and peptides or proteins that are resistant to shearing. In an embodiment, the composition that is formed is any one of the lipid vesicle compositions described herein.

In some embodiments, other lipid components are added at any one of the steps.

### EXAMPLES

### Example 1. Preparation of Multisome Lipid Vesicle Compositions of Hyaluronic Acid

Biphasic vesicles with multiple/synergistic penetration enhancers (Multisomes) were formulated with three different molecular weight hyaluronic acid, 250K, 50K and 10K (Creative PEGWorks, Chapel Hill, NC) at with either 1 mg/mL or 1.5 mg/mL concentration. For formulation development unlabelled HA was used. For the diffusion cell experiments the vesicles were prepared with labelled HAs (Rhodamine-HA250K, FITC-HA50K and FITC-HA-10K; Creative PEGWorks).

The general procedure for multisome preparation was as follows:
1) The oil phase and aqueous phase ingredients were weighed out in separate beakers.
2) Both beakers were heated to ˜70° C. to completely melt and incorporate all components.
3) The water phase was added to the oil phase, while stirring vigorously with a spatula to form an o/w crude emulsion, effectively yielding a homogenous milky solution (˜2 to 6 min) in the 70° C. water bath. The temperature of the solution was ˜55 to 65° C.
4) The formulation was batch processed using the LV 1 Microfluidizer or Nano DeBee homogenizer with Z5 module three times at C-20,000 psi.

Procedure for vesicle formation (applicable to all formulations):
1) The lipid phase components were weighed into a 20 mL glass vial.
2) The vial was heated to ˜70° C. in a water bath to completely melt and incorporate all components.
3) The water phase (System A) was added to the liquid phase while stirring vigorously for ˜10 to 20 min until the temperature of the solution was ˜60° C.

In some cases, the mixture was intermittently vortexed and heated for 5 sec/5 sec for 8-10 cycles until a uniform creamy lotion formed.

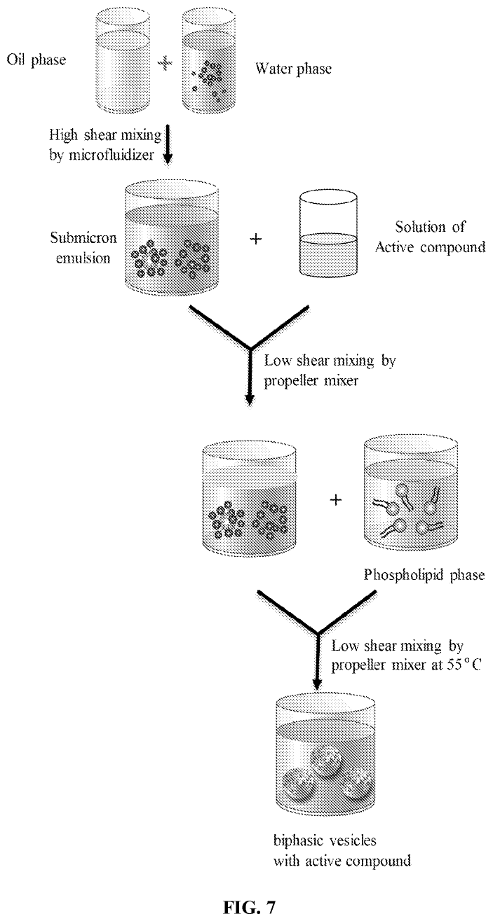

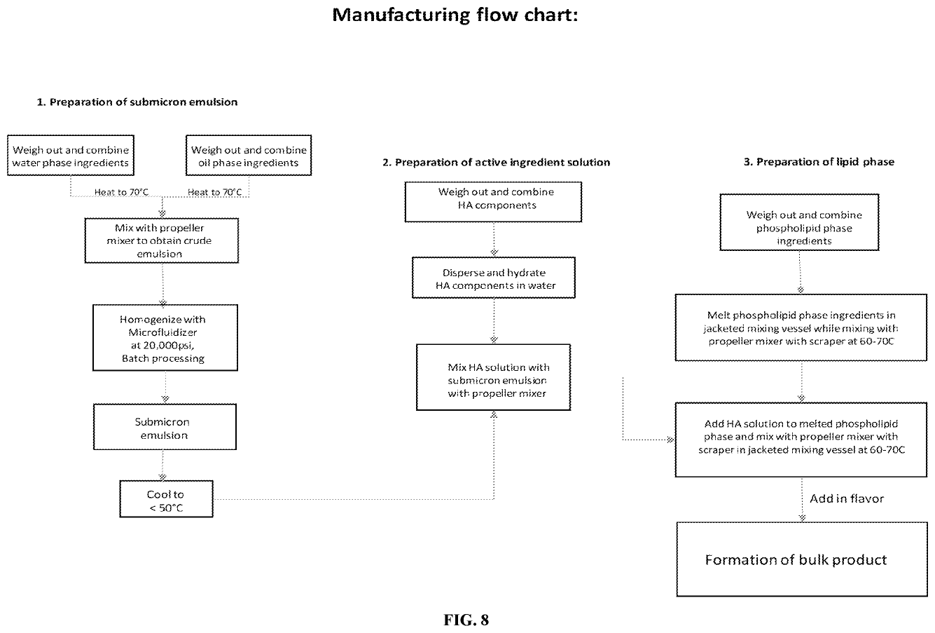

A pictorial representation of this process is shown in FIG. 7. A flow chart of this exemplary process is shown in FIG. 8.

The various lipid phases used throughout the experiments are described in Table A and the aqueous phases are described in Table B below.

TABLE A
Lipid Phases used in Formulations

| | | Concentration w/w (final |
|---|---|---|
| Lipid phase name | Ingredients | formulation) |
| F1SunL | Sunlipon 90H | 7% |
| | Cholesterol | 1.75% |
| | Propylene glycol | 7% |
| F1SunL1.5x | Sunlipon 90H | 10.5% |
| | Cholesterol | 2.7% |
| | Propylene glycol | 10.5% |
| F70SunL | Sunlipon 70H | 7% |
| | Cholesterol | 1.75% |
| | Propylene glycol | 7% |

TABLE B
Aqueous Phases used in Formulations

| Aqueous phases | | |
|---|---|---|
| (System A) | Ingredients | Concentration w/w |
| F4M | Labrafac CC | 5% |
| | Glyceryl monostearate NE | 1.2% |
| | Cetyl alcohol | 0.6% |
| | Syncrowax BB4 (beeswax) | 0.3% |
| | Arlasilk EFA | 5% |
| | Propylparaben | 0.05% |
| | Methylparaben | 0.15% |
| | Milli-Q Water | Qs to 100 |
| F2Gemini12312 | Labrafac CC | 5% |
| | Glyceryl monostearate NE | 1.2% |
| | Cetyl alcohol | 0.6% |
| | Syncrowax BB4 (beeswax) | 0.3% |
| | Dicationic (gemini) surfactant | 0.1% |
| | 12-3-12 | |
| | Tween 80 | 0.5% |
| | Propylparaben | 0.05% |
| | Methylparaben | 0.15% |
| | Milli-Q Water | Qs to 100 |
| F3Plys1-5K | Labrafac CC | 5% |
| | Glyceryl monostearate NE | 1.2% |
| | Cetyl alcohol | 0.6% |
| | Syncrowax BB4 (beeswax) | 0.3% |
| | Polylysine (Sigma) 1-5K | 0.05% |
| | Tween 80 | 0.5% |
| | Propylparaben | 0.05% |
| | Methylparaben | 0.15% |
| | Milli-Q Water | Qs to 100 |
| F5-COSM4 | Labrafac CC | 5% |
| | Glyceryl monostearate NE | 1.2% |
| | Cetyl alcohol | 0.6% |
| | Syncrowax BB4 (beeswax) | 0.3% |
| | Dimethicone 200CST | 1% |
| | Lipovol GBT | 0.2% |
| | Lipocol O2 | 1% |
| | Arlasilk EFA | 5% |
| | Euxyl9010 | 0.8% |
| | Spectrastat | 0.5% |
| | Milli-Q Water | Qs to 100 |
| F6-COSM5 | Labrafac CC | 5% |
| | Glyceryl monostearate NE | 1.2% |
| | Cetyl alcohol | 0.6% |
| | Syncrowax BB4 (beeswax) | 0.3% |
| | Dimethicone 200CST | 1% |
| | Super refined Brij O2 | 1% |
| | Arlasilk EFA | 5% |
| | Euxyl9010 | 0.8% |
| | Spectrastat | 0.5% |
| | Milli-Q Water | Qs to 100 |
| FA3-2 | Labrafac CC | 5% |
| | Glyceryl monostearate NE | 1.2% |
| | Cetyl alcohol | 0.6% |
| | SyncrowaxBB4 | 0.3% |
| | Everguard Polylysine (3-5K) | 0.2% |
| | Tween 80 | 0.5% |
| | Milli-Q Water | Qs to 100 |
| F6-COSM6 | Labrafac CC | 5% |
| | Glyceryl monostearate NE | 1.2% |
| | Cetyl alcohol | 0.6% |
| | Syncrowax BB4 (beeswax) | 0.3% |
| | Dimethicone 200CST | 1% |
| | Lipovol GBT | 0.2% |
| | Super refined Brij O2 | 1% |
| | Everguard Polylysine (3-5K) | 0.2% |
| | Arlasilk EFA | 5% |
| | Tween80 | 0.5% |
| | Euxyl9010 | 0.8% |
| | Spectrastat | 0.5% |
| | Milli-Q Water | Qs to 100 |
| F6-COSM6BN | Labrafac CC | 5% |
| | Glyceryl monostearate NE | 1.2% |
| | Cetyl alcohol | 0.6% |
| | Syncrowax BB4 (beeswax) | 0.3% |
| | Dimethicone 200CST | 1% |
| | Lipovol GBT | 0.2% |
| | Super refined Brij O2 | 1% |
| | Benzyl nicotinate | 0.5% |
| | Everguard Polylysine (3-5K) | 0.2% |
| | Arlasilk EFA | 5% |
| | Tween80 | 0.5% |
| | Euxyl9010 | 0.8% |
| | Spectrastat | 0.5% |
| | Milli-Q Water | Qs to 100 |

### Example 2. Methods of Analysis and Characterization

The following methods were used to characterize the formulations described in the following examples as well as the performance of the formulations.

Physicochemical Characterization—Organoleptic observations, light microscopy and confocal microscopy (Zeiss 710 confocal laser scanning microscope (CLSM)) were carried out to characterize the formulations. Confocal microscopy images of the formulations were obtained using a Zeiss LSM 710 CLSM using argon-laser 488 and HeNe-laser 543 lines for FITC (495/525) and Rhodamine (570/590), and either the Plan-Apochromat 20×/0.80 dry objective or the 63×/1.40 oil immersion objective. Optical zoom selection was applied in selected cases. Laser intensity, pinhole and gain settings were kept consistent between sample sets to enable comparison of relative fluorescence intensity measurements between samples. Images were captured and processed using the Zen 2009 software.

Size (hydrodynamic diameter) and polydispersity index and zeta (g) potential measurements were carried out on formulations using the Nano ZS Zetasizer (Malvern Instruments, Worcestershire, UK) which measures the hydrodynamic diameter of particles using dynamic light scattering (DLS). Aliquots of formulations were diluted 20× in water and 100 μL and 1000 μL of each formulation were prepared for size and zeta potential measurements, respectively. Measurements were carried out in triplicates.

In vitro diffusion cell studies—Full thickness human breast skin was obtained from female donors undergoing elective mammoplasty surgeries at the Royal University Hospital, University of Saskatchewan (Saskatoon, SK, Canada). Approval for skin collection was granted by the Human Ethics Committee at the University of Saskatchewan. The skin was collected within 2 h following surgery, trimmed of subcutaneous fat, and stored at −20° C. until use. In-line Bronaugh Flow-through diffusion cells with a 9 mm orifice diameter (0.63 cm²) were mounted on a water insulated cell warmer (PermeGear, Inc., Hellertown, PA) and set to a constant temperature of 32° C. Precut 1 cm2 skin sections were placed in the diffusion cells with the stratum corneum side facing up. Perfusion buffer (100 mM phosphate buffer with 0.05% Na-azide) at 37° C. was circulated through the lower half of the diffusion cells at a rate of 1 mL/h using a peristaltic pump. The surface of the skin was dosed with 0.1 mL of the formulations. Following 24 h incubation, the skin samples were removed from the cells, cleansed and processed for analysis.

Skin analysis—After removing the skin samples from the diffusion cells, first, the formulation remaining on the skin surface was removed. Each skin sample was subjected to a cleansing protocol and a tape stripping protocol to remove residual bound cream and the stratum corneum as follows: the skin sample was washed with 3×10 mL water, patted dry with a kimwipe and divided into 2 halves; one half of the skin was tape stripped two times (surface bound formulation removed), embedded into OCT compound on dry ice and cryosectioned The cryosections were examined by confocal microscopy.

Skin samples were cryosectioned with a Leica CM1850 cryostat into 10 μm sections. Sections on slides were left unstained. Confocal microscopy images of the skin sections were obtained using a Zeiss LSM 710 CLSM using argon-laser 488 and HeNe-laser 543 lines for FITC (495/525) and Rhodamine (570/590), and either the Plan-Apochromat 20×/0.80 dry objective or the 63×/1.40 oil immersion objective. Optical zoom selection was applied in selected cases. Laser intensity, pinhole and gain settings were kept consistent between sample sets to enable comparison of relative fluorescence intensity measurements between different treatments. Images were captured and processed using the Zen 2009 software.

The ‘no treatment’ skin sample was used to confirm gain and pinhole settings to exclude noise and autofluorescence background before the analysis of the subsequent treatment samples

### Example 3. Evaluation of Cationic Penetration Enhancers

The first strategy for formulating HA250K+HA10K 1 mg/mL combinations was to incorporate them into multisomes, i.e. next generation biphasic vesicles (synergistic enhancer type), with mono-, di- or polycationic building blocks to enhance the encapsulation and delivery of the negatively charged hyaluronic acid into skin layers. The components of these formulations are shown in Table C below.

TABLE C
Composition of formulations for delivery of
HA250 + 10 (1 mg/mL) using cationic excipients

| Formulation | Lipid phase | System A |
|---|---|---|
| F1 | F1SunL | F4M |
| F2 | F1SunL | F2G12312 |
| F3 | F1SunL | F3Plys1-5K |

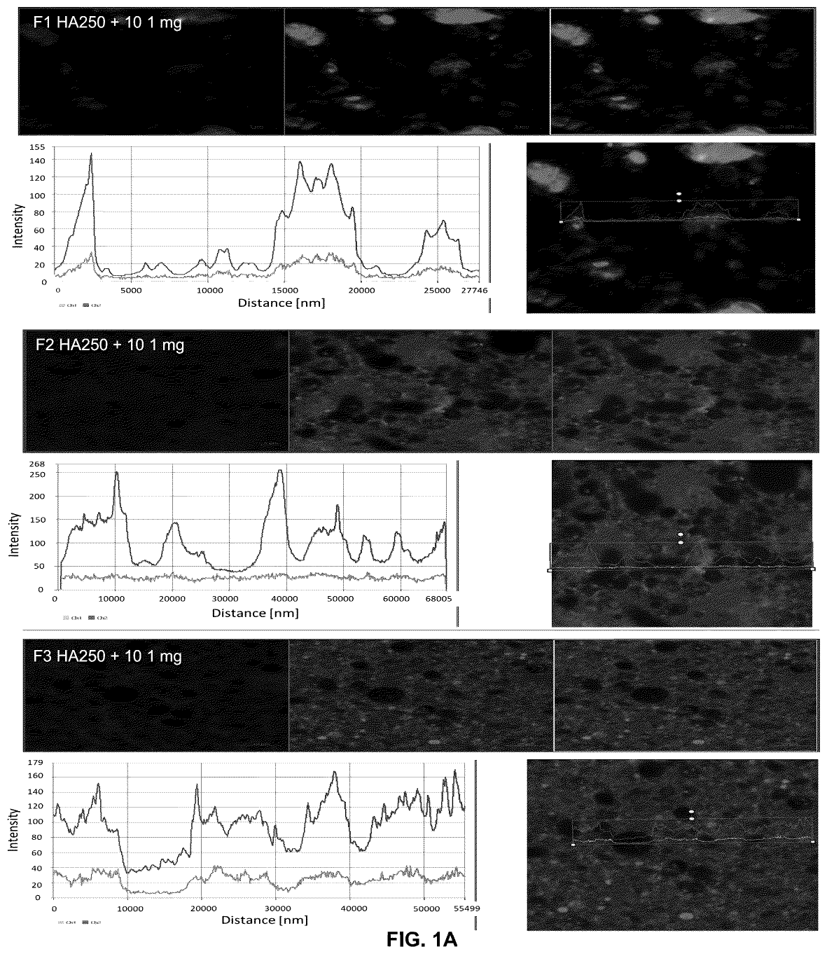

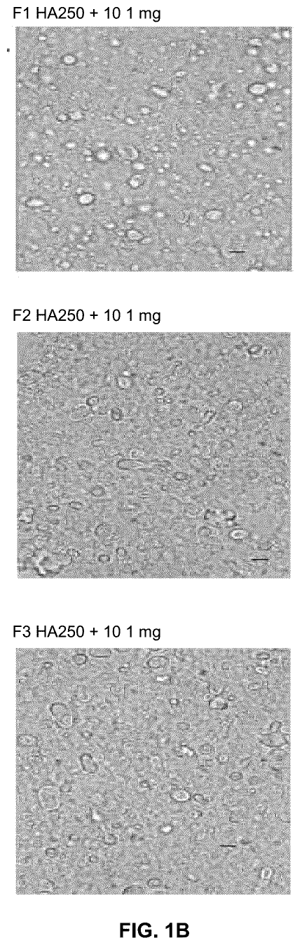

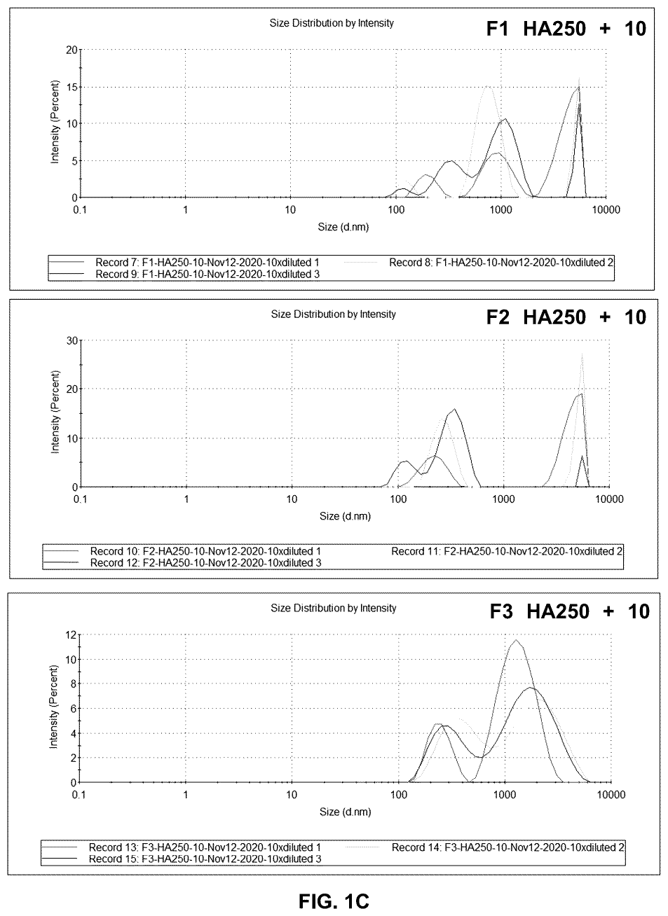

Confocal microscopic studies of the multisomes, showing the distribution of Rho-HA250K (red) and FITC-HA10K (green) fluorescence in the formulation, were used to analyze the HA encapsulation in the vesicles (FIG. 1 panel A images). The final concentration of HA in these samples was 1 mg/mL. Confocal microscopic profile tracings confirmed the association of red Rho-HA250K and green FITC-HA10K with the vesicles for formula F1, F2 and F3 (FIG. 1, panel A trace). The fluorescence intensity (FI) curves tracing the vesicles along the selected plane show the co-localization of the red and green fluorescence, indicating the co-encapsulation of the two different molecular weight HAs. Light microscopic images taken of formulations indicated the formation of multisomes (next generation biphasic vesicles) for each type of formulation (FIG. 1 panel B). Zetasizer studies for the System A (submicron emulsion component and the biphasic vesicles were carried out (FIG. 1 panel C). The formulations were shown to be polydisperse with vesicle sizes ranging generally between 0.3-10 μm. Zetasizer data show consistent results with the microscopic observations (FIG. 1 panel B). This is typical of multisomes. The zeta potential for F1, F2 and F3 were +33.6±0.6, +13.0±0.71 and −5.78±0.31, respectively. Similar data was observed for the other formulations described in the examples that follow (data not shown).

The physicochemical properties of the multisome formulations were assessed for color, consistency, and homogeneity. All formulations were lotion or cream consistency suitable for topical application. The formulations were physically stable showing no separation, sedimentation or other signs of stability issues for >3 mo of storage at 4° C. Microscopic observations confirmed that the multisomes remain intact and uniformly distributed during storage. Similar observations were made for the other formulations described in the examples that follow (data not shown).

The cryosections of human skin samples treated in vitro in diffusion cells with topical formulations containing fluorescence labelled HA were evaluated for the presence of fluorescent protein. The enhancement of delivery of (negatively charged) HA compounds is shown with three basic vesicle formulations utilizing three cationic vesicle building blocks (Table C).

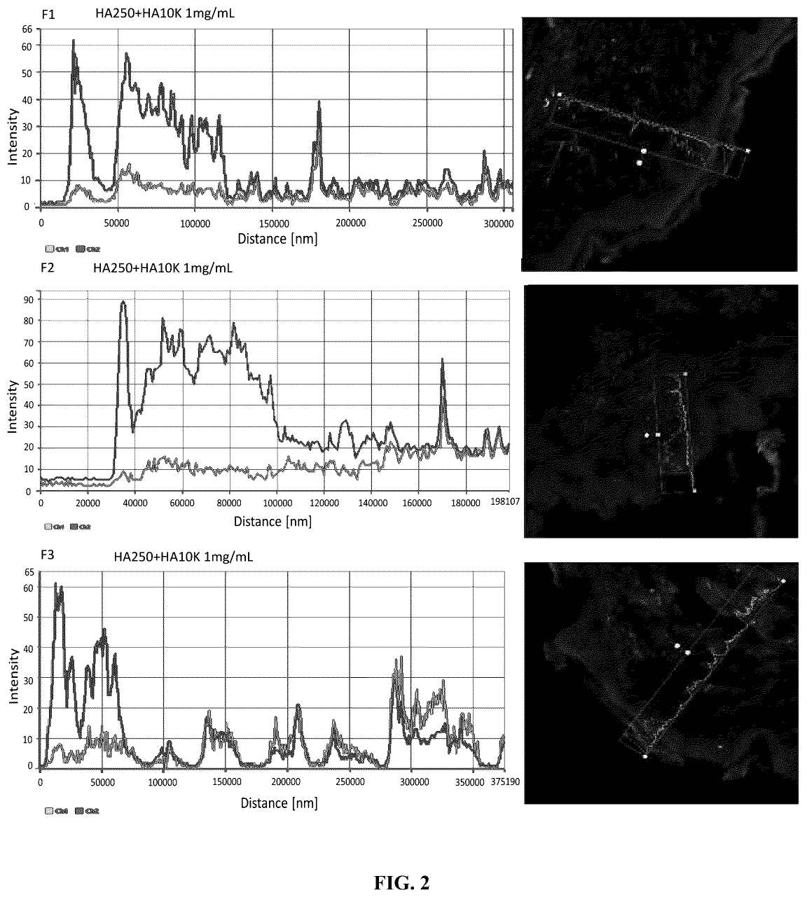

These studies indicated that all three cationic formulations increased HA delivery (Table D, FIG. 2). The order of enhancement followed the order dicationic>monocationic>polycationic formulations.

TABLE D
Analysis of the fluorescence intensity (FI) of skin
sections treated with F1, F2 and F3 formulations.

| | Average skin FI | | | Formulation FI |
|---|---|---|---|---|
| | (n = 3) | | Ratio | ratio |
| Formulation | Ch1 (FITC) | Ch2 (Rho) | Ch1/Ch2 ratio | Ch1/Ch2 ratio |
| F1 | 9 | 39 | 0.21 | 0.24 |
| F2 | 15 | 100 | 0.15 | 0.23 |
| F3 | 9 | 35 | 0.25 | 0.24 |

### Example 4. Evaluation of Hyaluronic Acid Concentration and Presence of Additional Cosmetic Components

Next, the effect of HA concentration was evaluated in the basic vesicle formulations. In order to assess this effect, 1 mg/m and 1.5 mg/mL of 250 kDa and 10 kDa hyaluronic acid (total weight of combined HA, equal mass of each molecular weight) 1 mg/mF and 1.5 mg/mL of 250 kDa and 50 kDa hyaluronic acid (total weight of combined HA, equal mass of each molecular weight) were prepared in the formulations provided in Table E. Also prepared were solution or gel formulations of HA according to formulations E11 and E12 of Table E made from SM hydroxypropyl methylcellulose (HPMC) gel.

TABLE E
Composition of formulations for delivery of
HA250 + 10 and HA250 + 50 combinations

| Formu- | | | |
|---|---|---|---|
| lation | | Lipid | |
| Number | Formulation | phase | System A |
| El | F4HA250 + 10 1 mg | F1SunL | F4M |
| E2 | F4HA250 + 10 1.5 mg | F1SunL | F4M |
| E3 | COSMF4HA250 + 10 1.5 mg | F1SunL | F5-COSM4 |
| E4 | F4HA250 + 50 1 mg | F1SunL | F4M |
| E5 | F4HA250 + 50 1.5 mg | F1SunL | F4M |
| E6 | COSMF4HA250 + 50 1.5 mg | F1SunL | F5-COSM4 |
| E7 | F3-2HA250 + 50 1.5 mg | F1SunL | FA3-2 |
| E8 | F1-F6-COSM6-HA250 + 10 1.5 mg | F1SunL | F6-COSM6 |
| E9 | F1-F6-COSM6-HA250 + 50 1.5 mg | F1SunL | F6-COSM6 |
| E10 | F1-F6-COSM6-HA250 + | F1SunL | F6-COSM6BN |
| | 50BN 1.5 mg | | |

| — | Gel Formulation | Vehicle |
|---|---|---|
| E11 | Gel-HA250 + 10 1.5 mg | 1% HPMC gel |
| E12 | Gel-HA250 + 50 1.5 mg | 1% HPMC gel |

Upon administration to human skin as provided in Example 2, the results shown in Table F below were obtained. Increasing concentration in HA250/10K or HA250/50K total concentration from 1 mg/mL to 1.5 mg/mL vesicle formulation increased delivery, as shown by comparing formula E1 vs E2 and formula E4 vs E5 (Table F), especially evident from the increase of the HA250K component. Further optimization to obtain cosmetic vesicle formulations, indicated that these changes did not affect delivery, see formula E2 vs E3 and E5 vs E6 (Table F).

Comparing formulas E7, E8, E9 and E10 (Table F) indicate that these compositions did not achieve delivery enhancement compared to the other formulations. In formula E7 and E10 (Table 7), the strategy of using polycationic and monocationic agents together decreased delivery compared to using monocationic agent alone. The gel formulations (formula E11 and E12 of Table F) where the HA250/10K or 250/50K was incorporated in ‘free’ (not encapsulated) form showed very low/negligible delivery levels.

All multiphasic vesicle formulations enhanced delivery compared to HA in solution or gel formulations. Formulation design differences of multiphasic vesicles indicated that HA delivery can be modulated.

TABLE F
Analysis of fluorescence intensities (FI) in
skin sections treated with HA formulations

| | | Average skin FI | | |
|---|---|---|---|---|
| Formu- | | (n = 3) | | Ratio |
| lation | | Ch1 | Ch2 | Ch1/Ch2 |
| Number | Formulation | (FITC) | (Rho) | ratio |
| E1 | F4HA250 + 10 1 mg | 15 | 45 | 0.33 |
| E2 | F4HA250 + 10 1.5 mg | 15 | 75 | 0.2 |
| E3 | COSMF4HA250 + 10 1.5 mg | 12 | 70 | 0.17 |
| E4 | F4HA250 + 50 1 mg | 30 | 85 | 0.35 |
| E5 | F4HA250 + 50 1.5 mg | 20 | 100 | 0.2 |
| E6 | COSMF4HA250 + 50 1.5 mg | 18 | 120 | 0.15 |
| E7 | F3-2HA250 + 50 1.5 mg | 10 | 10 | 1 |
| E8 | F1-F6-COSM6-HA250 + 10 | 10 | 15 | 0.66 |
| | 1.5 mg | | | |
| E9 | F1-F6-COSM6-HA250 + 50 | 10 | 35 | 0.28 |
| | 1.5 mg | | | |
| E10 | F1-F6-COSM6BN -HA250 + 50 | 8 | 60 | 0.13 |
| | 1.5 mg | | | |
| E11 | Gel-HA250 + 10 1.5 mg | 7 | 15 | 0.47 |
| E12 | Gel-HA250 + 50 1.5 mg | 12 | 15 | 0.8 |

Also assessed were multisome compositions having additional cosmetic properties, including formulations which included Lipovol GBT (tribehenin) or benzyl nicotinate. The effect on transdermal penetrations of these components on hyaluronic acids having combination molecular weights of 250/10 kDa and 250/50 kDa was assessed. The formulations tested are shown below in Table G.

TABLE G
Composition of formulations for delivery of HA250 +
10 and HA250 + 50 combinations (total HA concentration
1.5 mg/mL) with optimized cosmetic properties

| Formulation | | | |
|---|---|---|---|
| Number | Formulation | Lipid phase | System A |
| | HA250K + 10K | | |
| G1 | Formula F1-F5-COSM4- | F1SunL1.5x | F5-COSM4 |
| | HA250 + 10 | | |
| G2 | Formula F1-F5-COSM4- | F1SunL1.5x | F5-COSM4 |
| | HA250 + 10 + BN | | |
| G3 | Formula F1-F6-COSM5- | F1SunL1.5x | F6-COSM5 |
| | HA250 + 10 | | |
| G4 | Formula F1-F6-COSM5 - | F1SunL1.5x | F6-COSM5 |
| | HA250 + 10 + BN | | |
| | HA250K + 50K | | |
| G5 | Formula F1-F5-COSM4- | F1SunL1.5x | F5-COSM4 |
| | HA250 + 50 | | |
| G6 | Formula F1-F5-COSM4- | F1SunL1.5x | F5-COSM4 |
| | HA250 + 50 + BN | | |
| G7 | Formula F1-F6- COSM5 | F1SunL1.5x | F6-COSM5 |
| | HA250 + 50- | | |
| G8 | Formula F1-F6-COSM5- | F1SunL1.5x | F6-COSM5 |
| | HA250 + 50 + BN | | |
| | Other formulations | | |
| G9 | Formula F4-HA250 + 50-F70- | F70SunL | F4M |
| | 1.5 mg | | |
| G10 | Formula F4-HA250 + 50 + | F1SunL1.5x | F4M |
| | 10-1.5 mg | | |

Upon administration to human skin as provided in Example 2, the results shown in Table H below were obtained.

TABLE H

| | | Average skin FI | | |
|---|---|---|---|---|
| | | (n = 3) | | Ratio |
| | | Ch1 | Ch2 | Ch1/Ch2 |
| | Formulation | (FITC) | (Rho) | ratio |
| G1 | Formula F1-F5-COSM4- | 12 | 70 | 0.17 |
| | HA250 + 10 | | | |
| G2 | Formula F1-F5-COSM4- | 15 | 124 | 0.12 |
| | HA250 + 10 + BN | | | |
| G3 | Formula F1-F6-COSM5- | 9 | 55 | 0.16 |
| | HA250 + 10 | | | |
| G4 | Formula F1-F6-COSM5 - | 18 | 112 | 0.16 |
| | HA250 + 10 + BN | | | |
| G5 | Formula F1-F5-COSM4- | 15 | 74 | 0.2 |
| | HA250 + 50 | | | |
| G6 | Formula F1-F5-COSM4- | 14 | 58 | 0.24 |
| | HA250 + 50 + BN | | | |
| G7 | Formula F1-F6- COSM5 | 12 | 70 | 0.17 |
| | HA250 + 50- | | | |
| G8 | Formula F1-F6-COSM5- | 14.2 | 100.8 | 0.14 |
| | HA250 + 50 + BN | | | |
| G9 | Formula F4-HA250 + 50-F70- | 16 | 35 | 0.45 |
| | 1.5 mg | | | |
| G10 | Formula F4-HA250 + 50 + 10- | 15 | 17 | 0.88 |
| | 1.5 mg | | | |

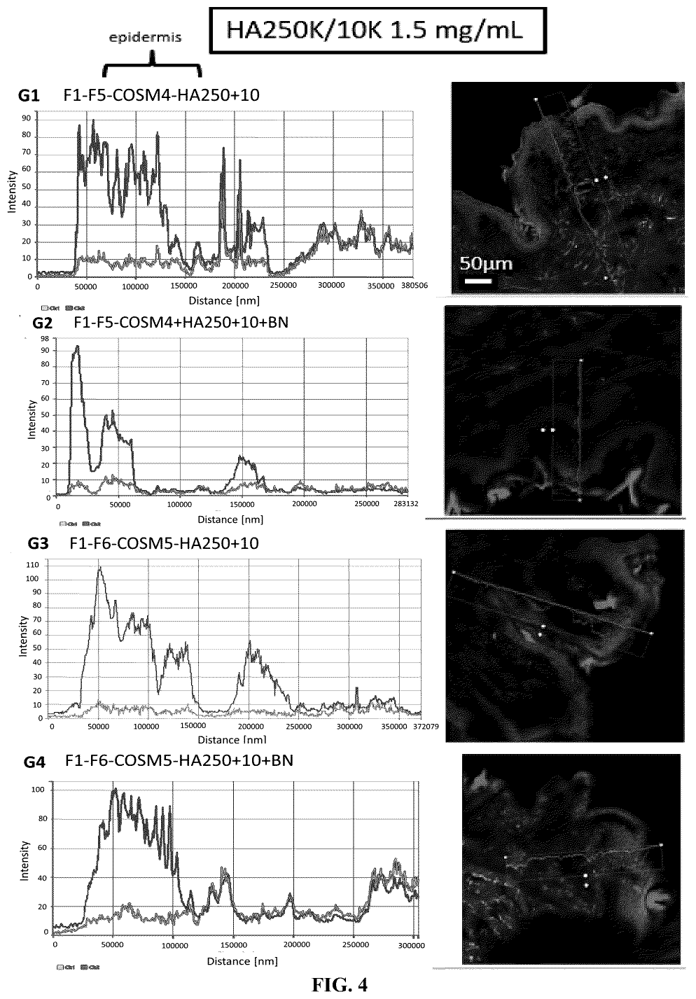

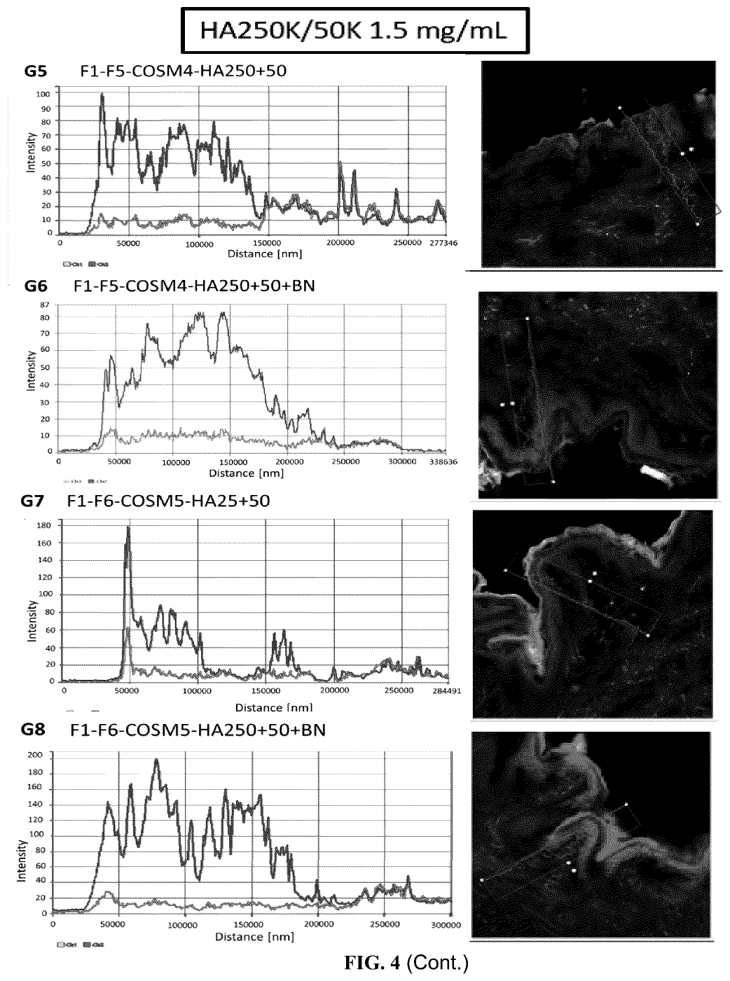

Comparing delivery efficiencies from compositions with ingredients added for cosmeceutical effects indicated that tribehenin and benzyl nicotinate (BN) influenced the delivery. For example, for the delivery of HA250/10K formulations G1 vs G2 (Table H) and formulas G3 and G4 (Table H and FIG. 4) indicates that BN enhanced delivery whether T was present or absent.

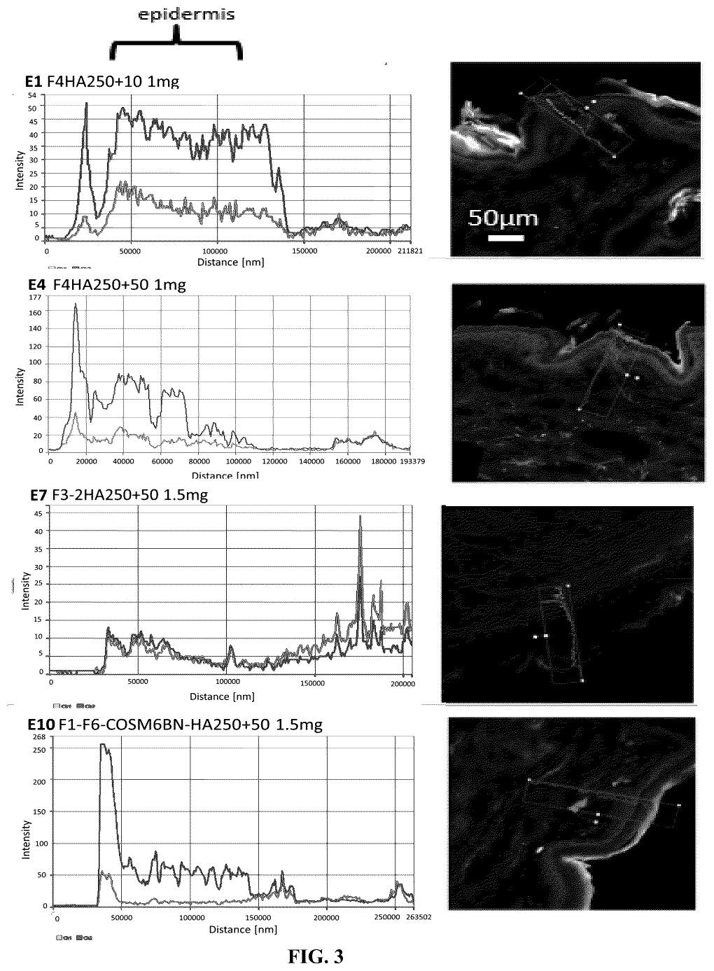

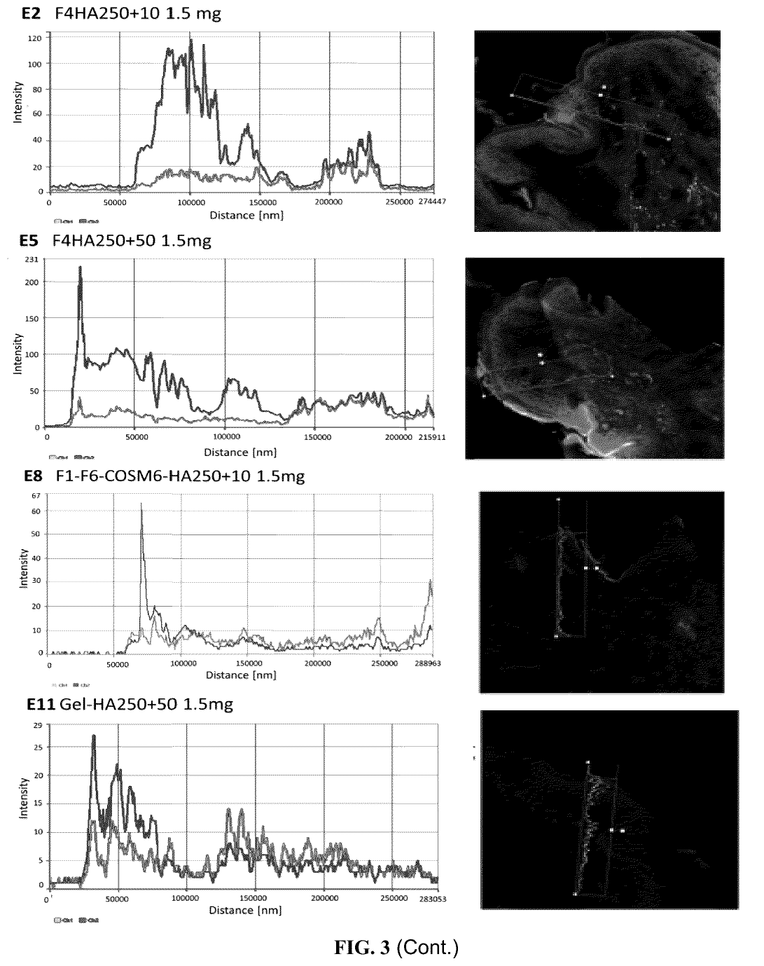

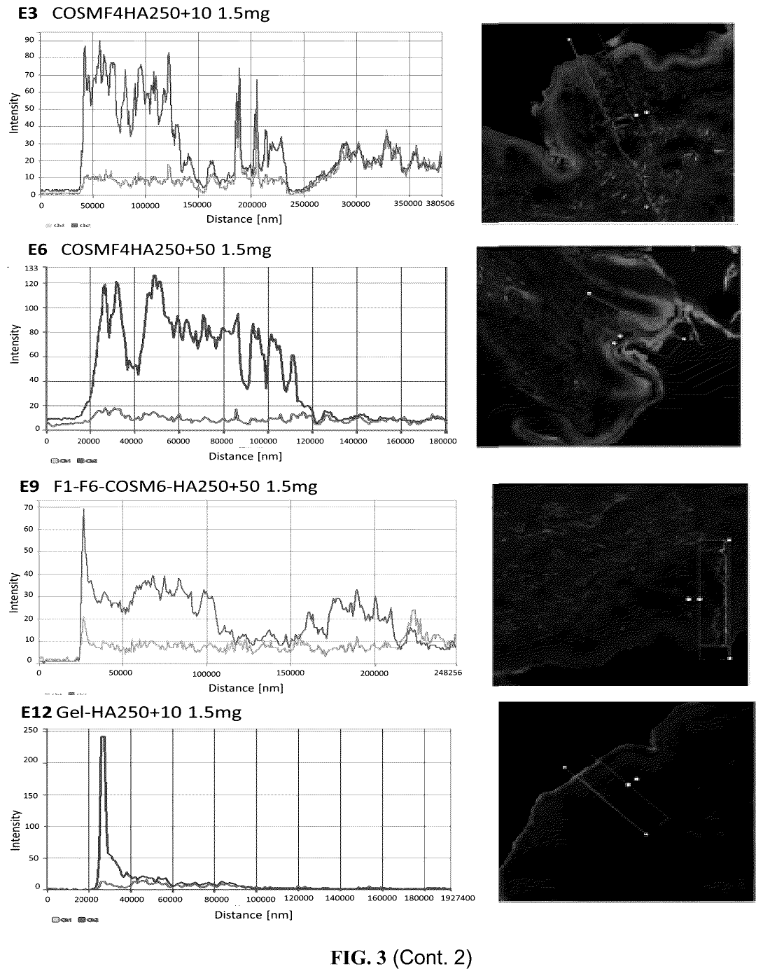

When comparing formulas G5 and G7 (Table H) and formulas G6 and G8 (Table H and FIG. 4) indicates that the presence or absence of tribehenin alone (without BN) did not affect delivery, but BN enhanced delivery when tribehenin was not present which may mean that tribehenin inhibits the effect of BN enhancing delivery. This is also noticeable when comparing formulas G7 and G8 (Table H and FIG. 3) which shows that in the absence of tribehenin, BN increases delivery.

When comparing formulas G4 and G8 (Table H and FIG. 4) it is noted that using BN but no tribehenin is the preferable formulations for delivering both HA250/10K or 250/50K combinations, with an overall 200% increased efficiency.

Another formulation composition tested but found to be less effective in delivering HA250/50K was formula G9 (Table H and FIG. 4) when the phospholipid component was replaced with another type of phospholipid. Additionally, an increase in concentration of lipid phase components and the inclusion of each of 250 kDa, 50 kDa, and 10 kDa MW hyaluronic acid (formula G10; Table H) was also found to be less effective. Compare, e.g., G10 of Table H with E2 and E5 of Table F.

Additionally, there was no substantial difference in the delivery of the two different HA mwt combinations 250/10K or 250/50K from the different sets of equivalent formulations with the same overall composition. See, e.g., G1 vs. G5 and G3 vs. G7.

In Table F and H, the ratio of Ch1/Ch2 fluoresce values are also shown for the skin samples treated with various formulations. The similar ratio indicates that the delivery of FITC-HA10 or FITC-HA50K and Rho-HA250K is similar to the original ratio of these two actives in the multisome formulations, that is they are simultaneously delivered at the same extent. Ratios that are lower indicate that the HA250K component delivery is further enhanced relative to the smaller polymer.

### Example 5. Evaluation of a Multisome Formulation to Enhance Penetration of a Nicotinic Acetylcholine Receptor Peptide Antagonist (Conotoxin Peptide Analog)

The objective of this work was to explore the skin penetration properties of a conotoxin peptide analog (hereafter designated “C7 peptide”; Glo Pharma) from next generation biphasic vesicle formulations (multisomes). The C7 peptide has an amino acid sequence similar to that of SEQ ID NO: 3, which is a naturally occurring conotoxin peptide antagonist of muscle-type nicotinic acetylcholine receptors. As with all of the peptide provided herein (e.g., SEQ ID Nos: 1-52 and 60-99), the C7 peptide possesses similar properties with the native conotoxin of SEQ ID NO:3 for purposes of formulation a lipid vesicle delivery composition (e.g., similar size, conformation, charge, etc.). Thus, it is expected that lipid vesicle compositions which work for the C7 peptide will similarly work for the other peptides provided herein.

Three multisome type vesicles (F6A-C7, F1B-C7, F1C-C7) were formulated with C7 peptide at loading concentration of 2 mg/mL, and compared to C7 peptide solution. The formulations were characterized for physicochemical properties.

In vitro diffusion cell study was conducted with multiphasic vesicle formulations F6A-C7, F1B-C7, F1C-C7 and transdermal fractions were collected for further analysis by mass spectrometry at Climax Laboratories.

Overall, the results show that suitable C7 peptide containing multiphasic vesicles cream formulations can be prepared for intra/transdermal delivery.

The specific objectives for these experiments were to assess the skin delivery of C7 peptide from various formulations developed as follows: 1) The uptake of C7 peptide through human skin was evaluated in experiments using Bronaugh type in-line diffusion cells and human breast skin. The skin samples were treated with formulations containing C7 peptide and the penetration through the skin was evaluated in the transdermal fractions by mass spectrometry; 2) As a reference free C7 peptide solution was used for comparison, and blank vehicles were used as controls 3) Transdermal fractions were collected hourly for analysis 4) The fractions were pooled and concentrated by Pall filtration and shipped for analysis at Climax Laboratories, Inc. (San Jose, CA).

Materials and Methods

Formulations: Biphasic vesicles with multiple/synergistic penetration enhancers (multisomes)—Five different vesicles were formulated. From these, three formulations were selected for testing. For formulation development, C7 peptide (Anaspec, code:74337, Lot #1958617) was used. For the diffusion cell experiments multisomes without peptide and with 2 mg/mL of C7 peptide were used. The diffusion cell dose was 0.1 g formulation with 0.2 mg peptide. The pH of formulations was between 6.2-6.7.

Physicochemical characterization—Organoleptic observations, light microscopy and confocal microscopy (Zeiss 710 confocal laser scanning microscope (CLSM; Carl Zeiss GmH, Germany) were carried out to characterize the formulations.

Size (hydrodynamic diameter), polydispersity index and zeta (g) potential measurements were carried out on formulations F6A-C7, F1B-C7, F1C-C7 prepared with non-labelled C7 peptide using the Zetasizer NanoZS (Malvern Instruments, Worcestershire, UK) which measures the hydrodynamic diameter of particles using dynamic light scattering (DLS). Aliquots of 80 uL formulations were assessed for particle size distribution and subsequently diluted 10× with water for zeta potential measurements. Measurements were carried out in triplicates.

In vitro diffusion cell study—Full thickness human breast skin was obtained with permission from female donors undergoing elective mammoplasty surgeries at the Royal University Hospital, University of Saskatchewan (Saskatoon, SK, Canada). Approval for tissue collection was granted by the University of Saskatchewan Human Ethics Committee. The skin was collected within two hours following surgery, trimmed of subcutaneous fat, and stored at −20° C. until use.

C7 peptide absorption from formulations into excised human skin in vitro was evaluated using 9 mm diameter Bronaugh-type teflon flow-through diffusion cells (PermeGear, Inc., Hellertown, PA) with an exposed surface area of 0.636 cm². The cell holder was maintained at 32° C. by a circulating water bath heater. Degassed phosphate-buffered saline (PBS) buffer with 0.05% sodium-azide pH 7.2, maintained at 37° C., was used as a perfusion fluid with a flow rate of 1 mL/h. Skin samples were removed from the freezer and cut into about 1 cm×1 cm square pieces and mounted into the diffusion cells epidermis facing up. F6A-C7, F1B-C7, F1C-C7 multisome formulations with C7 peptide or control blank formulations (100 μL per cell) were applied to the skin at t=0 and the cells were covered with a teflon cap to provide occlusion. Treatment was performed for 24 hours. Transdermal fractions were collected into 3 mL tissue culture tubes using a programmed fraction collector to collect the hourly fractions for a total of 24×1 mL/cell.

Transdermal fraction analysis—The transdermal fractions were collected hourly for analysis for 24h. For each cell twentyfour 1 mL fractions were collected and labelled 1/1h, ½h, ⅓h . . . 1/24h, etc. The samples were sent for analysis to Glo/Climax Analytical Labs.

The skin samples from the diffusion cell study were cleansed by the usual protocol to remove residual bound cream, i.e. after the skin samples were removed from the diffusion cells and washed with 3×10 mL water, and patted dry with a kimwipe. The cleansed skin discs are stored at −20° C.

Results and Discussion

Multisome Formulation Optimization and Characterization

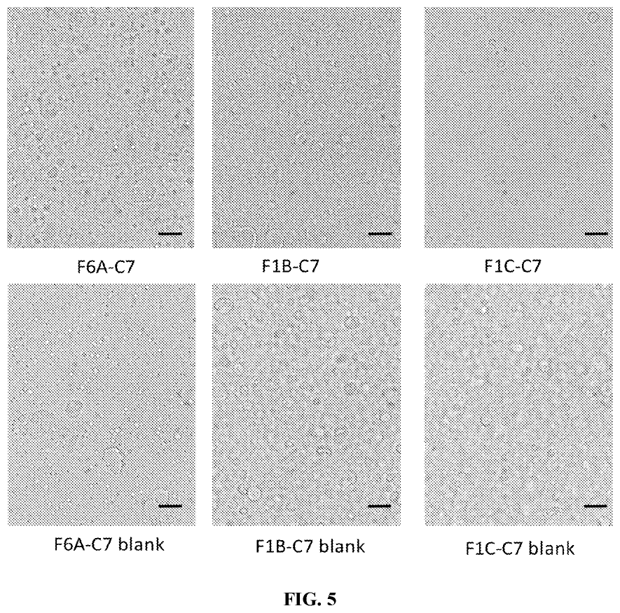

Table I and Table J show the compositions of the formulations developed with and without C7 peptide. Initially Phospholipon 90H (soybean phosphatidylcholine) was used to prepare the formulations. Then subsequently, it was replaced by Sunlipon 90H (sunflower phosphatidylcholine) (SunL) based on organoleptic and physicochemical observations. Light microscope images of the resulting lipid vesicle formulations are shown in FIG. 5.

TABLE I
Formulation compositions for the delivery of C7 peptide

| | |
|---|---|
| Formulation | |
| Batch size: 0.5 g | Compositional information |

| C7 peptide: 2 mg/mL | System A | Lipid phase |
|---|---|---|
| Formula F6A-C7 | F4M | F2SunL |
| Formula F1B-C7 | F1 | F3SunL |
| Formula F1C-C7 | F1 | F4SunL |

TABLE J
Formulation compositions

| Lipid phases | Ingredients | Concentration w/w |
|---|---|---|
| F2 | Phospholipon 90H | 7.00% |
| | MLL(monolauroyl lysine) | 2.00% |
| | Oleth-2 (Lipocol-O2) | 1.00% |
| | Cholesterol | 1.75% |
| | Propylene glycol | 7.00% |
| F3 | Phospholipon 90H | 7.00% |
| | Cholesterol | 1.75% |
| | Lipopeg-2DL | 1.00% |
| | Propylene glycol | 7.00% |
| F4 | Phospholipon 90H | 7.00% |
| | Cholesterol | 1.75% |
| | Oleth-2 (Lipocol-O2) | 1.00% |
| | Propylene glycol | 7.00% |
| F2SunL | Sunlipon 90H | 7.00% |
| | MLL(monolauroyl lysine) | 2.00% |
| | Oleth-2 (Lipocol-O2) | 1.00% |
| | Cholesterol | 1.75% |
| | Propylene glycol | 7.00% |
| F3SunL | Sunlipon 90H | 7.00% |
| | Cholesterol | 1.75% |
| | Lipopeg-2DL | 1.00% |
| | Propylene glycol | 7.00% |
| F4SunL | Sunlipon 90H | 7.00% |
| | Cholesterol | 1.75% |
| | Oleth-2 (Lipocol-O2) | 1.00% |
| | Propylene glycol | 7.00% |

Table K summarizes the organoleptic properties, physical stability particle size ranges and physical stability. All formulations were lotion or cream consistency suitable for topical application. The formulations were physically stable showing no separation, sedimentation or other signs of stability issues for >1 mo of storage at 4° C.

TABLE K
Organoleptic properties of vesicle formulations

| | |
|---|---|
| Formulation | Physical stability |

| | | |
|---|---|---|
| Formula F6A-C7 | White lotion | Stable >6 mo at 4° C. |
| Formula F1B-C7 | White lotion | Stable >6 mo at 4° C. |
| Formula FIC-C7 | White cream | Stable >6 mo at 4° C. |

These formulations were shown to be polydisperse with vesicle sizes ranging generally between 0.3-5 μm as shown on the light microscopic images (FIG. 1 and Table 4). The microscopic observations confirmed the formation of multisomes with the typical biphasic vesicle morphology and the uniform distribution of vesicles throughout the formulation for both C7 peptide-containing and blank (no peptide) formulations.

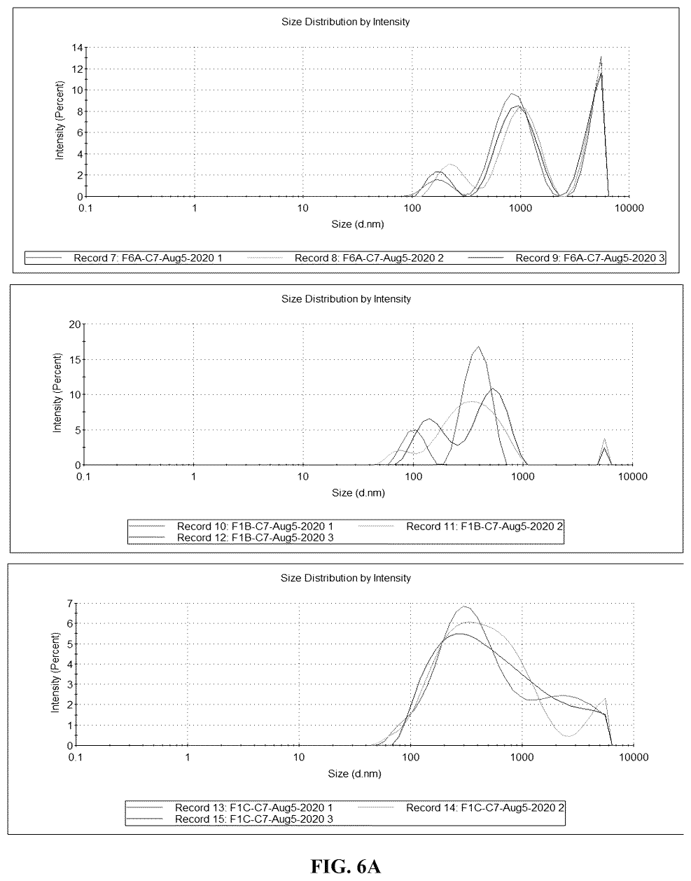

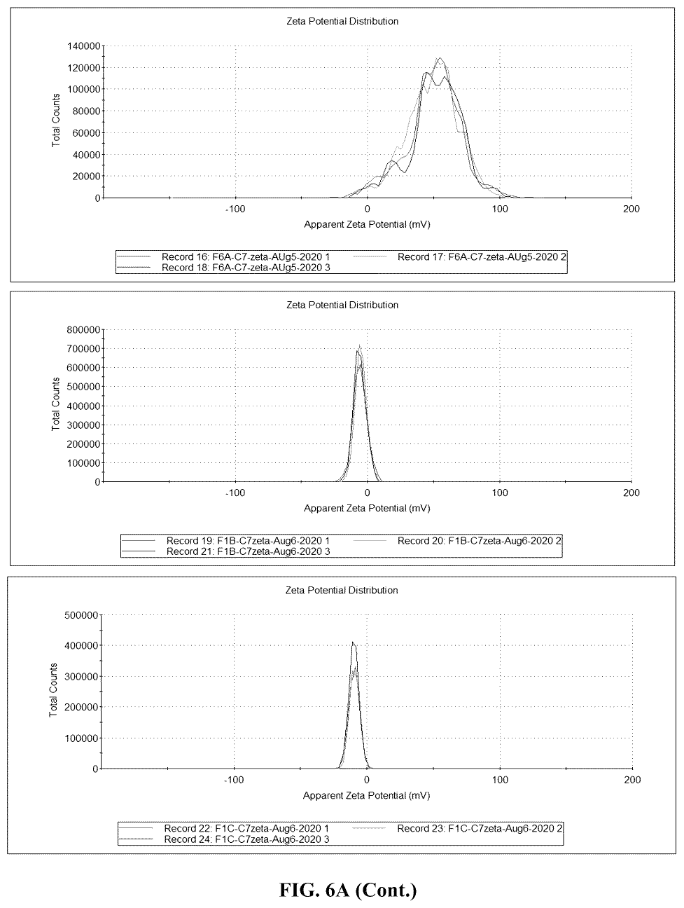

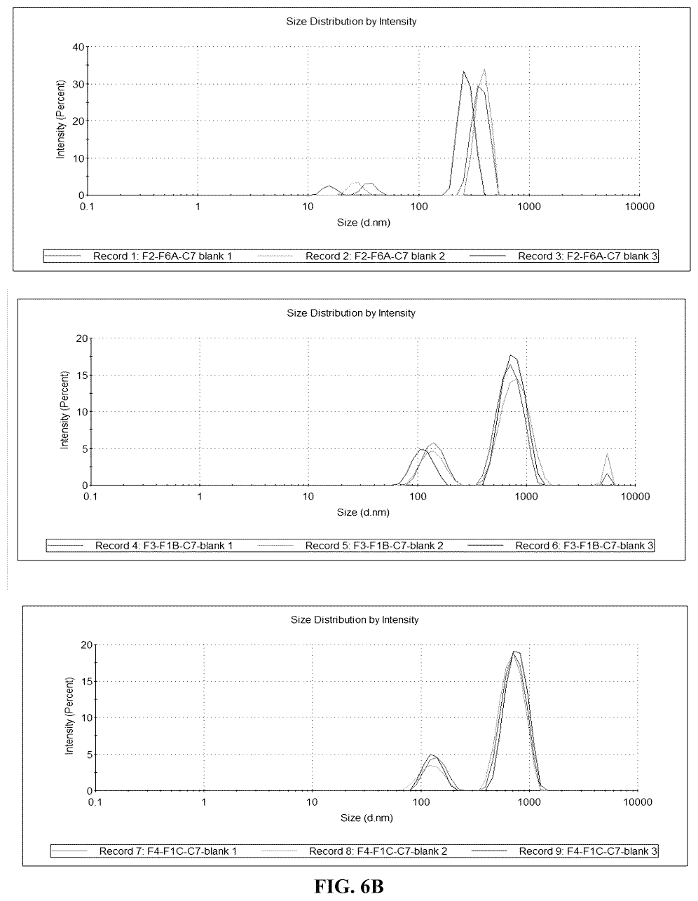

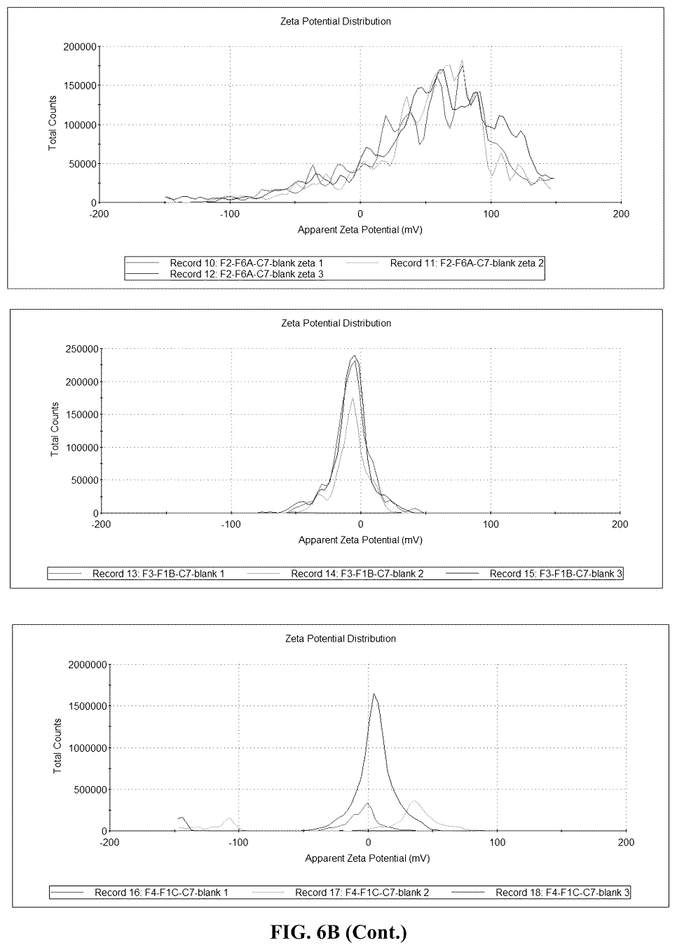

Each of the C7 peptide-containing formulations were similar with respect to size distribution compared to their respective blank formulations, but overall the blank formulations had narrower size distribution compared to the peptide formulations (FIG. 6A and FIG. 6B)

Zetasizer data showed consistent results with the microscopic observations (FIG. 6B) This is typical of multisomes. The zeta potential of F6A-C7 was positive and the other two, F1B-C7 and F1C-C7, were negatively charged (Table L and FIG. 6A and FIG. 6B)

TABLE L
Particle size analysis and zeta potential of multiphasic
vesicle formulations made with unlabeled C7

| | Average particle size range (μm) | Zeta potential |
|---|---|---|
| Formulation | (microscopy) | (mV) |
| Formula F6A-C7\hat{ } | Polydisperse | +50.0 ± 1.06 |
| | 0.3-5 | |
| Formula F1B-C7* | Polydisperse | −5.38 ± 0.384 |
| | 0.1-10 | |
| Formula F1C-C7* | Polydisperse | −9.40 ± 0.231 |
| | 0.4-8 | |
| Formula F6A-C7\hat{ } | Polydisperse | +35.3 ± 0.87 |
| blank | 0.3-5 | |
| Formula F1B-C7* | Polydisperse | −7.63 ± 0.224 |
| blank | 0.1-10 | |
| Formula F1C-C7* | Polydisperse | −3.37 ± 0.264 |
| blank | 0.4-8 | |

\hat{ }prepared with System A F4M: 177.0 ± 8.255 nm; +27.3 ± 3.13 mV
*Prepared with System A F1: 160.3 ± 6.038; −5.70 ± 0.295

Lipid vesicle formulations were prepared with C7 concentrations of 2 mg/mL and administered to skin samples at 200 microgram of skin sample. Blank versions of each formulation were prepared as controls, and a mg/mL solution of the C7 peptide in water was prepared as an additional control. Each formulation was tested in triplicates and blank formulations, untreated skin and C7 peptide solution as a free, non-encapsulated peptide were used for background fractions for the analysis.

Diffusion Cell Study—Transdermal Delivery of C7

In this study the transdermal fractions were collected for further analysis by mass spectrometry by Climax Analytical Laboratories.

These studies showed that all three multisome formulations delivered the C7 peptide deeply into and through the human skin in vitro. Among these multisome formulations, Formula F6A-C7 and F1B-C7 delivered about two times higher amount of C7 peptide compared to F1C-C7.

The total amount (Qt (24h)) of C7 peptide delivered through the 9 mm diameter skin disk treated in the diffusion cells (0.636 cm² surface area) was 599.62±265.62, 600.46±402.77 and 276.56±111.47 ng/24h for F6A-C7, F1B-C7 and F1C-C7, respectively, corresponding to 0.3, 0.3 and 0.14% delivery rates for each formulation. The Qt per unit surface area of the skin, i.e. ng/cm² and percent C7 peptide delivers are shown in Table M.

TABLE M
Transdermal delivery of C7 peptide from multisome formulations

Total amount (Qt) C7 peptide
delivered through the skin

| | ng/24 h/cm² | Average dose |
|---|---|---|
| Formulation | mean ± s.d. (n = 3) | absorbed % |
| Formula F6A-C7 | 942.80 ± 418.10 | 0.47 |
| Formula F1B-C7 | 944.12 ± 633.28 | 0.47 |
| Formula F1C-C7 | 434.84 ± 175.27 | 0.22 |

These experiments indicated that the enhancement level with the synergistic components was as follows for C7 peptide: PEFA/Oleth-2=Tween 80/Span 40/PEG-4-dilaurate>Tween 80/Span 40/Oleth-2.

### Example 6. Evaluation of the Safety and Efficacy of a Muscle-Type nAChR Peptide Antagonist Compared to Placebo for Treatment of Facial Wrinkles

A lipid vesicle formulation of a muscle-type nAChR peptide antagonist of the disclosure is tested for safety and efficacy compared with placebo in the treatment of facial rhytids (skin wrinkles) and glabellar frown lines in a randomized, double-blind human clinical trial. Patients (50 in each group) are treated with an amount of the muscle-type nAChR peptide antagonist, or placebo (blank lipid vesicle) applied to the bilateral forehead and frown line areas on Day 1.

Primary outcome: Percentage of Participants Achieving a Score of None or Mild by Investigator-Assessment of Facial Wrinkle Scale With Photonumeric Guide (FWS) in Forehead Lines at Maximum Eyebrow Elevation.

On Day 30, the severity of the patient's forehead lines at maximum eyebrow elevation using the 4-point Facial Wrinkle Scale with Photonumeric Guide (FWS): 0=none, 1=mild, 2=moderate or 3=severe is assessed. The percentage of participants with a score of none or mild is determined.

Primary outcome: Percentage of Participants Achieving a Score of None or Mild by Subject-Assessment of Facial Wrinkle Scale With Photonumeric Guide (FWS) in Forehead Lines at Maximum Eyebrow Elevation.

Also on Day 30, the patients assess the severity of their forehead lines at maximum eyebrow elevation using the 4-point Facial Wrinkle Scale with Photonumeric Guide (FWS): 0=none, 1=mild, 2=moderate or 3=severe and the percentage of participants with a score of none or mild is determined.

Secondary outcome: Percentage of Participants Achieving Satisfied or Very Satisfied by Subject Assessment of Satisfaction of Appearance of Forehead Lines (participant assessment).

On Day 30, participants rate their overall satisfaction with the appearance of the forehead line area using a 5-point scale: 1=very unsatisfied, 2=unsatisfied, 3=neutral, 4=satisfied or 5=very satisfied. The percentage of participants with a rating of satisfied or very satisfied is determined.

Secondary outcome: Percentage of Participants With a=1 Grade Improvement from Baseline by Investigator-Assessed FWS in Forehead Lines at Rest.

At baseline and on Day 30, the Investigator assesses the severity of the patient's forehead lines at rest using the 4-point FWS: 0=none, 1=mild, 2=moderate or 3=severe. The percentage of participants with a=1 grade improvement from baseline is determined.

Secondary outcome: Percentage of Participants With a=1 Grade Improvement from Baseline by Subject-Assessed FWS in Forehead Lines at Rest.

At baseline and on Day 30, the participant assesses the severity of their forehead lines at rest using the 4-point FWS: 0=none, 1=mild, 2=moderate or 3=severe. The percentage of participants with a=1 grade improvement from baseline is determined.

### Example 7. Evaluation of the Safety and Efficacy of Topical Application of a Lipid Vesicle Composition Comprising Hyaluronic Acid Compared to Placebo for Enhancement of Lip Characteristics

A lipid vesicle formulation of hyaluronic acid of the disclosure is tested for safety and efficacy compared with placebo in the application to lips in a randomized, double-blind human clinical trial. An amount of hyaluronic acid containing lipid vesicle formulation, or placebo (blank lipid vesicle) is applied to the upper and lower lips of subjects (50 in each group) on Day 1.

Primary outcome: An increase in lip fullness of at least one grade on Medicis Lip Fullness Scale (MLFS) at 14 and 30 days post-usage. The MLFS is determined by an independent dermatologist: The MLFS is a validated, 5-point scale of lip fullness (1=very thin; 2=thin; 3=medium; 4=full; 5=very full).

On Day 14, the fullness of the subject's lips using the 5-point MLFS: 1=very thin; 2=thin; 3=medium; 4=full; 5=very full is assessed and compared to baseline (before usage with the lipid vesicle formulation). The number of subjects with an improvement of at least 1 MLFS level is determined.

On Day 30, the fullness of the subject's lips using the 5-point MLFS is assessed again to determine the longevity of the effect after usage. The MLFS score is compared to baseline and the Day 14 result. The number of subjects with an improvement of at least 1 MLFS level is determined.

Secondary outcome: Percentage of Participants Achieving Satisfied or Very Satisfied by Subject Assessment of Satisfaction of Appearance of Lips (participant assessment).

On Day 30, participants rate their overall satisfaction with the appearance of their lips using a 5-point scale: 1=very unsatisfied, 2=unsatisfied, 3=neutral, 4=satisfied or 5=very satisfied. The percentage of participants with a rating of satisfied or very satisfied is determined.

Secondary outcome: Assessment of the efficacy and durability of the filler, by a dermatologist: Dermatologist opinions about the effectiveness and durability of the filler effect is assessed, using 5-point Investigator's Global Assessment (IGA) Scale: 1=Worse, 2=Mildly Improved, 3=Improved, 4=Much Improved, 5=Very Much Improved.

An independent dermatologist compares images of the subject's lips taken at Day 0 (prior to usage), Day 14, and Day 30. The number of subjects showing a rating of Improved, Much Improved, or Very Much Improved is assessed for Day 14 and Day 30 and compared.

### Example 8. Evaluation of the Safety and Efficacy of Topical Application of a Lipid Vesicle Composition Comprising Hyaluronic Acid for Enhancement of Lip Characteristics

A lipid vesicle formulation of hyaluronic acid as described herein (e.g. a lipid vesicle formulation of hyaluronic acid as described in Example 1) was tested for safety and efficacy in the application to lips in a human clinical trial. An amount of hyaluronic acid containing lipid vesicle formulation was applied to the upper and lower lips of subjects (55 total) for five days. The subjects were 35 and older, and included a variety of ethnic backgrounds and skin types.

The subjects applied five layers of the formulation twice daily (morning and night). Each layer of the formulation was reapplied once the previous layer was fully absorbed into the upper and lower lips of the subject. The increase in height of the upper and lower lip of each subject was measured before and after the five days, as measured from the baseline between the upper and lower lip.

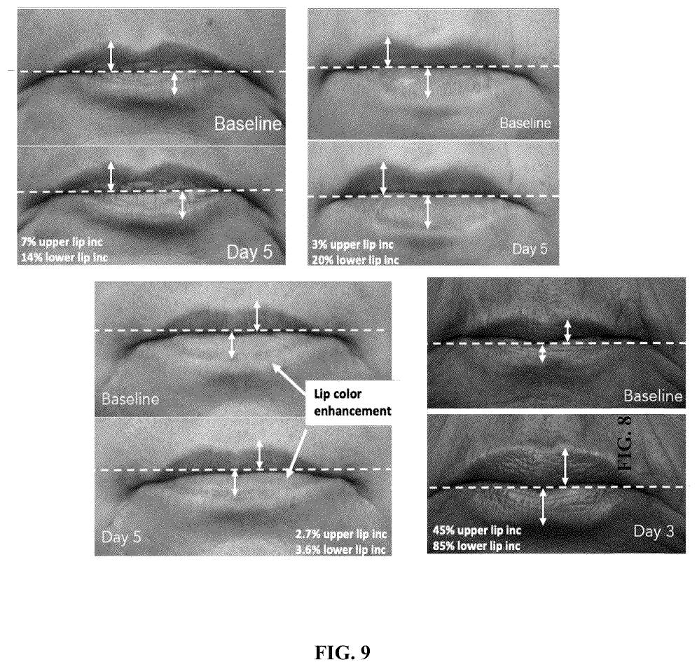

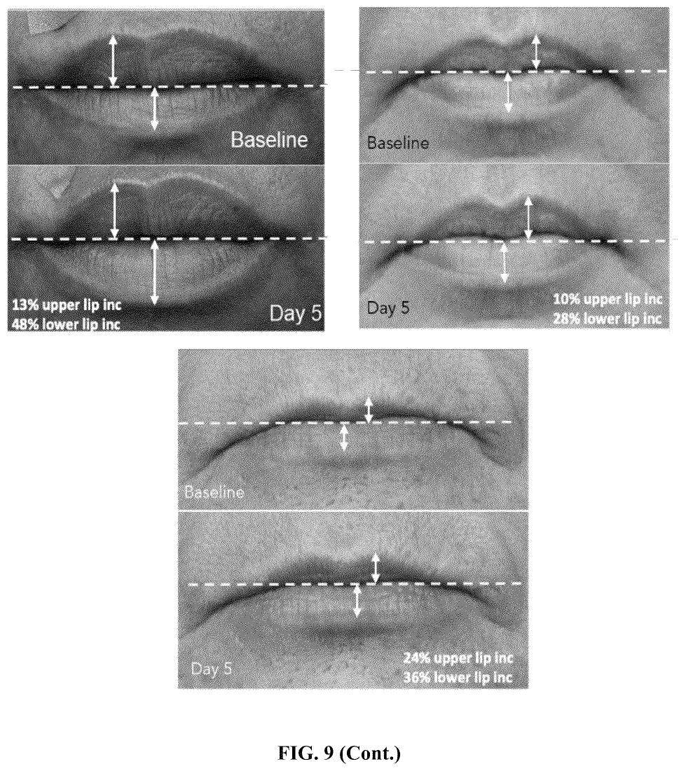

FIG. 9 shows the results after five days of application, where subjects showed noticeably fuller lips across ethnicities and skin types. As shown, subjects had anywhere from 2.7% to 45% increase in upper lip height as measured from the baseline and anywhere from 3.6% to 85% increase in lower lip height as measured from the baseline. Further, subjects experienced smoother and more colorful lips after the five days of applying the formulation.

### Example 9. Lipid Vesicle Formulation Development

The starting compositions for the development of lipid vesicle formulations were based on the previous F6A-C7 formula, as well as F-C10-5 and F-C10-6 formulas, whose main enhancer combination are shown in Table N.

TABLE N
Enhancer combinations of F6A-C7, F-C10-5 and F-C10-6 formulas

| Formula | System A | Lipid phase |
|---|---|---|
| F6A-C7 | Arlasilk EFA | Oleth-2; cholesterol |
| F-C10-5 | Arlasilk PTM + Capryol90 | Phytosterol MM |
| F-C10-6 | Arlasilk EFA + Oleth2 | cholesterol |

Using these formulations as starting points, variations of system A (water phase) were developed (Table O), as well as variations of the phospholipid phase (Table P).

TABLE O
Enhancer combinations in system A (water phase)

| System ACOSM | Enhancer Ingredient combination |
|---|---|
| System A 15 | Arlasilk EFA + Oleth-2 |
| System A 15-2 | Arlasilk EFA + Oleth-2 |
| System A 15-3 | Arlasilk EFA |
| System A 15-4 | Arlasilk EFA + Oleth-2 + Tween80 + Span40 |
| System A 16 | Arlasilk PTM + Oleth-2 + Capryol90 + Oleyl alcohol |
| System A 17 | Arlasilk EFA + Oleth-2 + LexfeelN350MB |
| System A 18 | Arlasilk EFA + Oleth-2 + isopropyl myristate |
| System A 16B | Arlasilk EFA + Oleth-2 + Capryol90 + Oleyl alcohol |
| System A 16B2 | Arlasilk EFA + Arlasilk PTM + Oleth-2 + |
| | Capryol90 + Oleyl alcohol |
| System A 16ST | Arlasilk PTM + Oleth-2 + Capryol90 + Oleyl |
| | alcohol + Tween 80 + Span40 |
| System A 19 | Arlasilk EFA + Oleth-2 + Capryol90 |

TABLE P
Enhancer combinations in phospholipid phase

| Lipid phase | Ingredient combination |
|---|---|
| F1 | Sunlipon 90H + cholesterol |
| F5P | Sunlipon 90H + phytosterol MM |
| F5PB | Sunlipon 90H + phytosterol MM + Botanisil SP-360 |
| F5P2.6 | Sunlipon 90H + phytosterol MM2.6% |
| F1B | Sunlipon 90H + cholesterol + Botanisil SP-360 |
| F8P | Sunlipon 90H + phytosterol MM + Oleth-2 |
| F8 | Sunlipon 90H + cholesterol + Oleth-2 |
| F8PB | Sunlipon 90H + phytosterol MM + Oleth-2 + |
| | Botanisil SP-360 |
| F8B | Sunlipon 90H + cholesterol MM + Oleth-2 + |
| | Botanisil SP-360 |
| F9 | Sunlipon 90H + cholesterol + Softouch CG6097 |
| F9P | Sunlipon 90H + phytosterolMM + Softouch CG6097 |
| F10 | Sunlipon 90H + cholesterol + MLL |
| F10P | Sunlipon 90H + phytosterolMM + MLL |

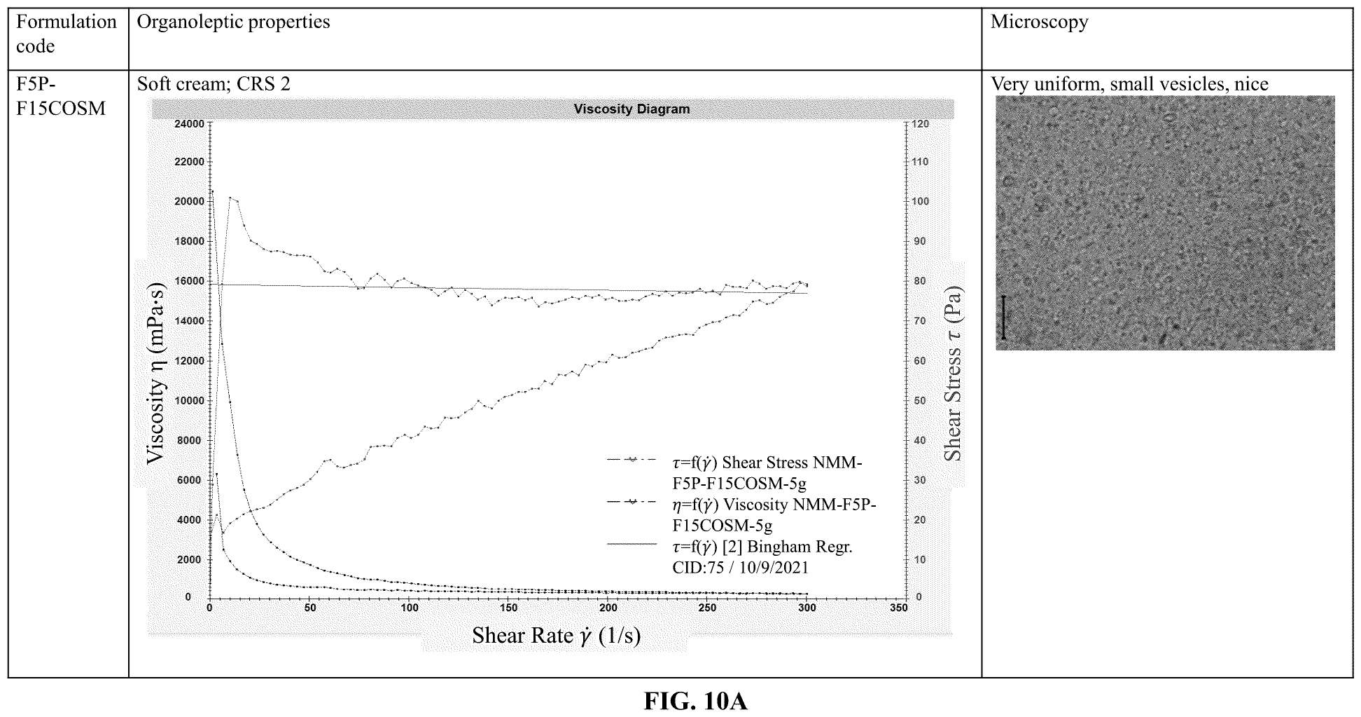

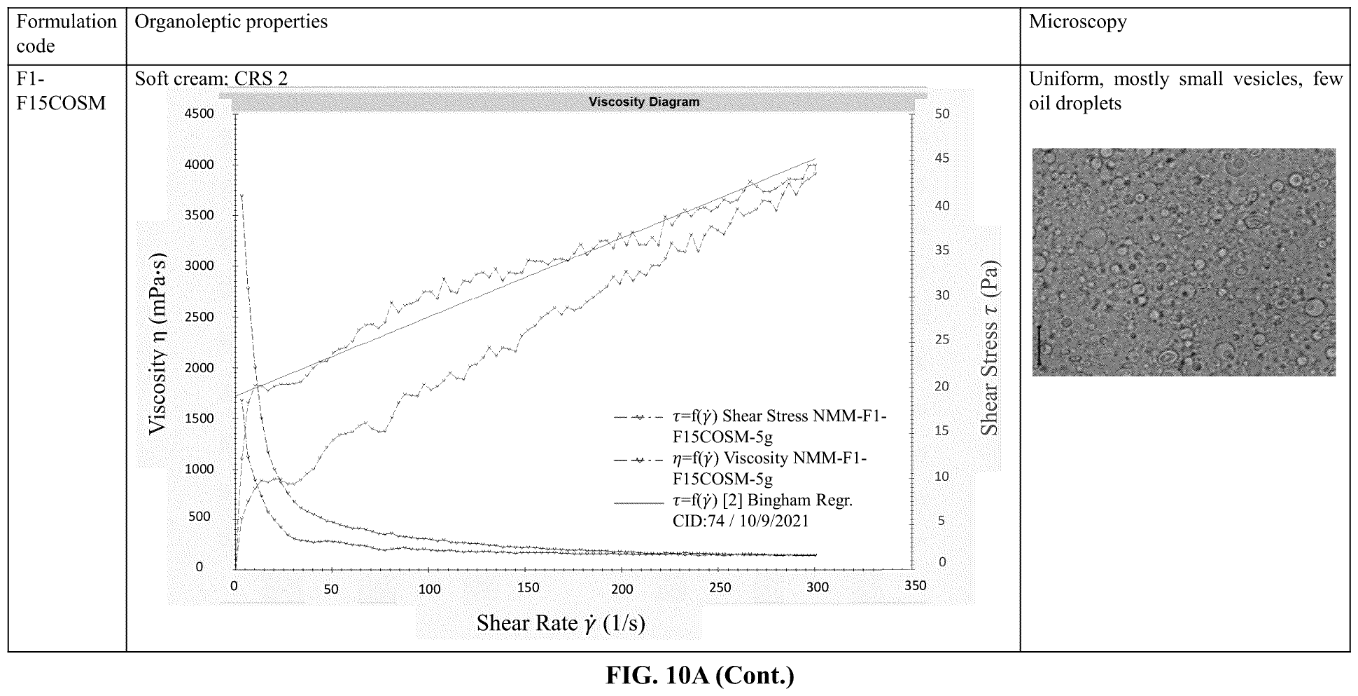

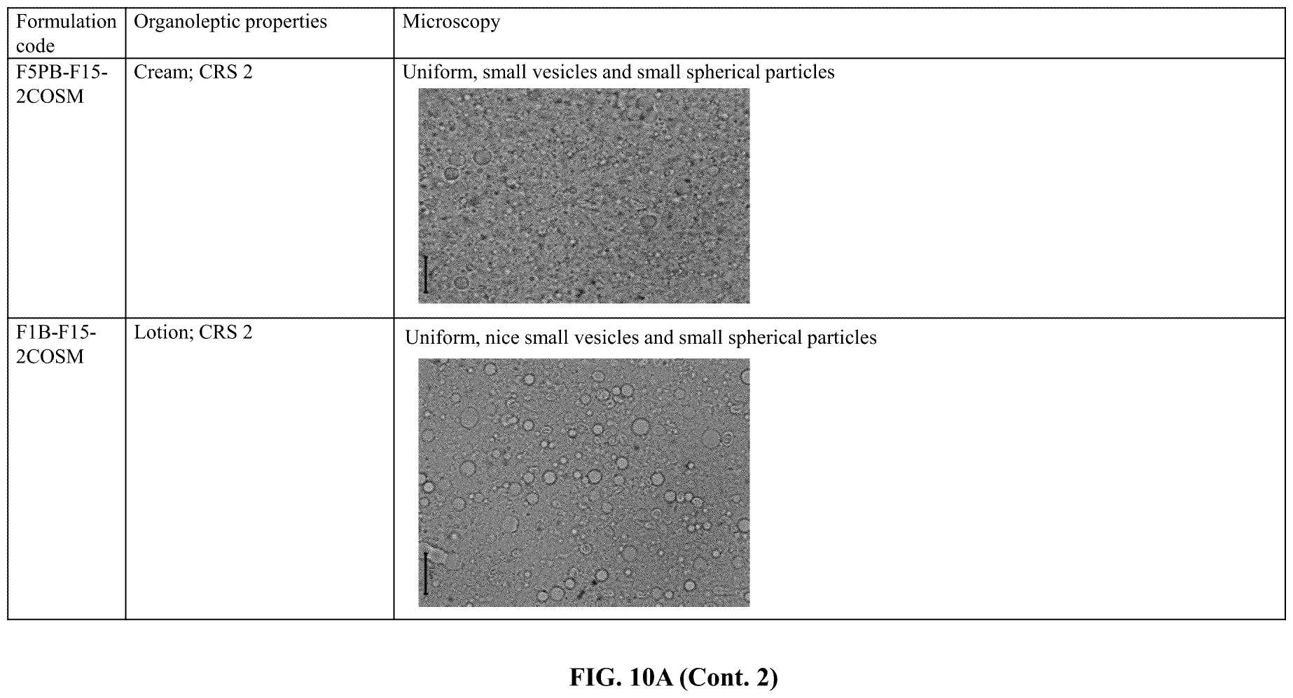

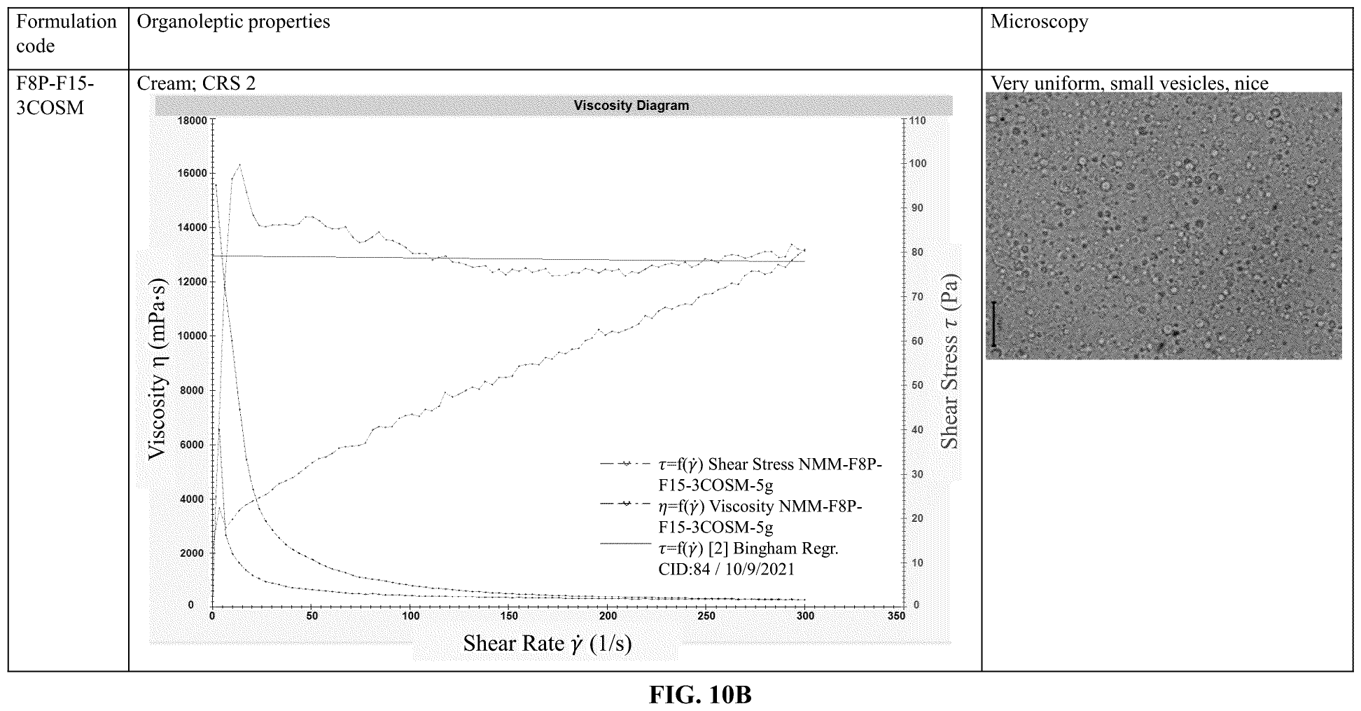

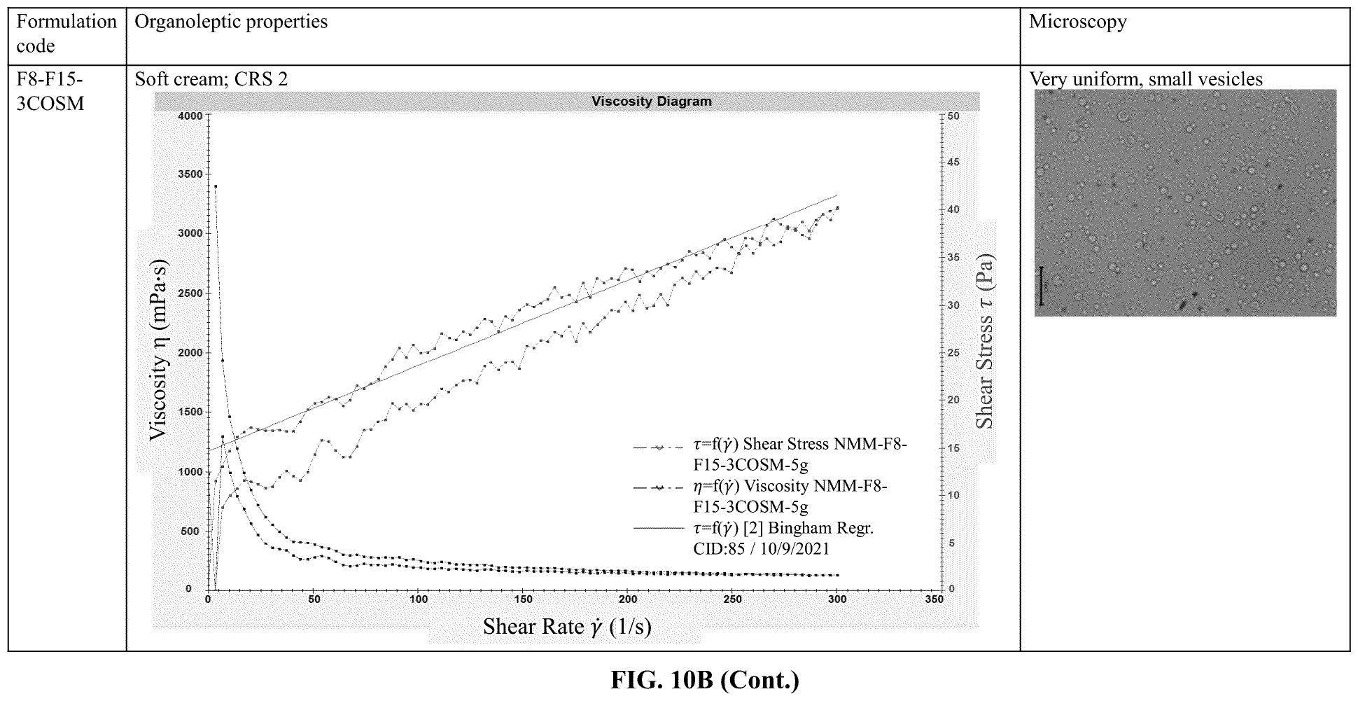

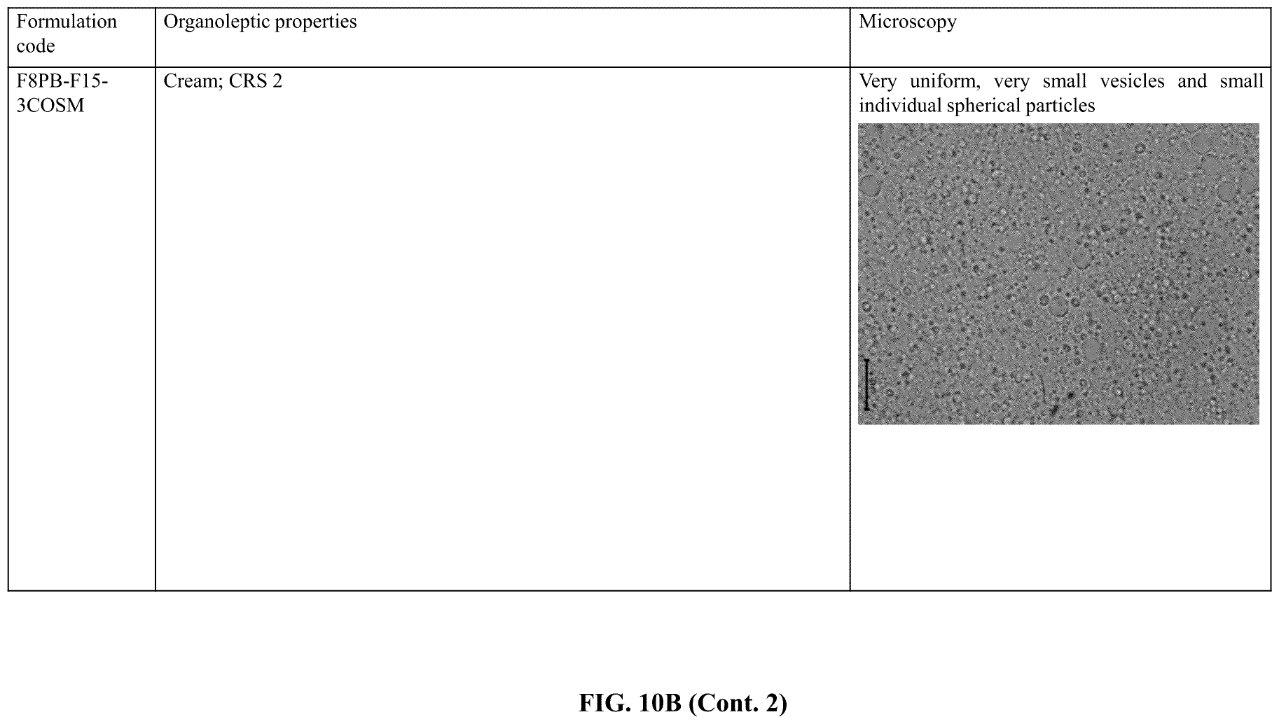

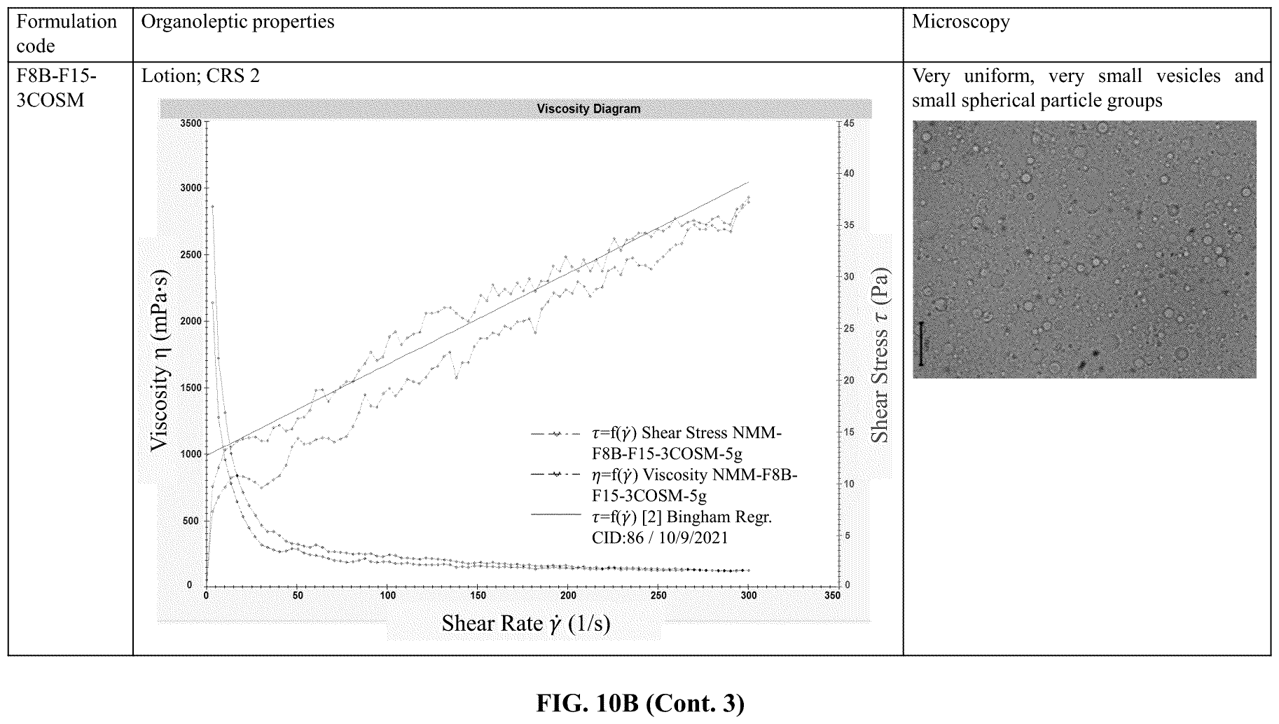

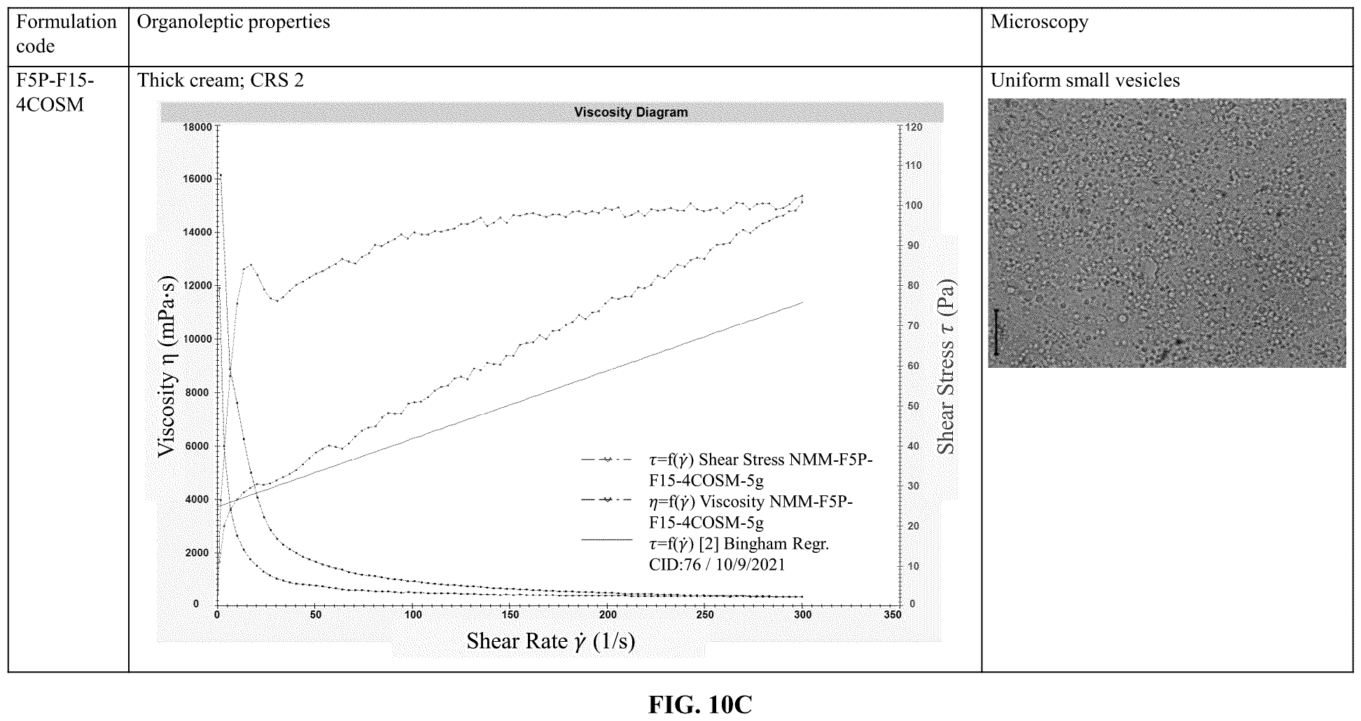

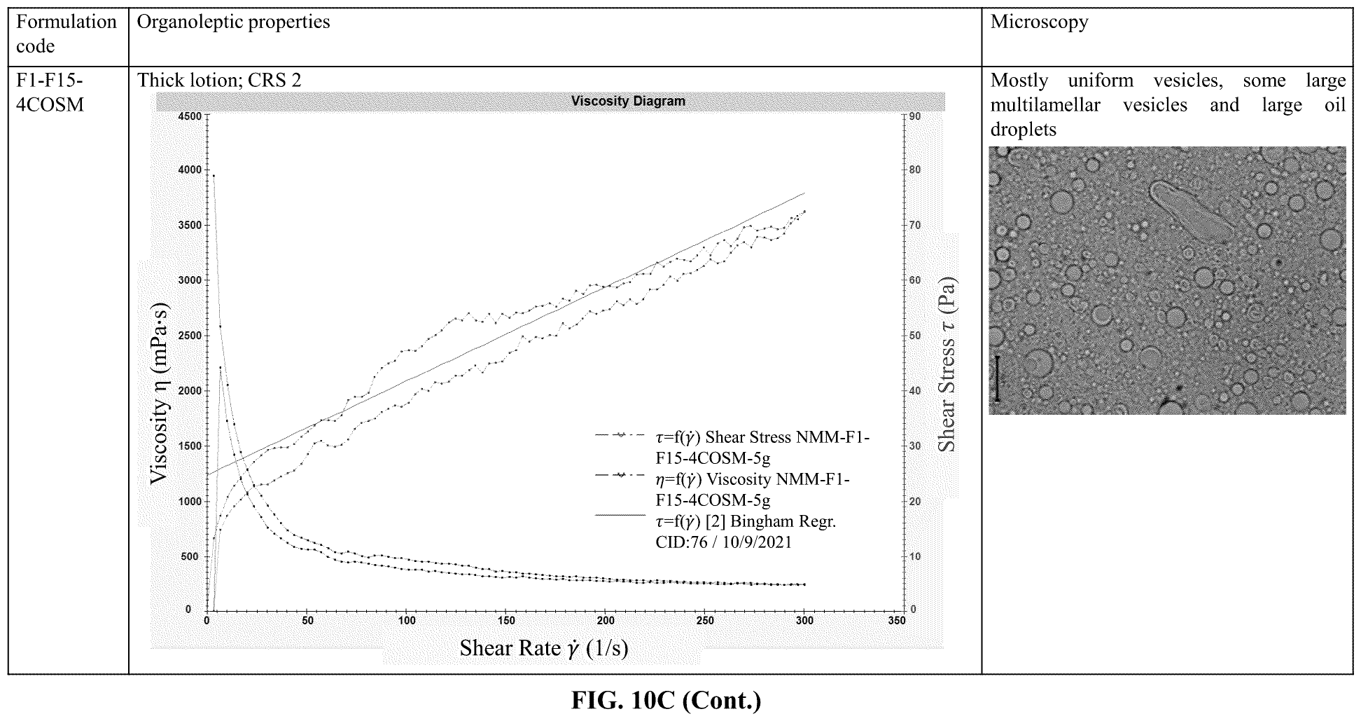

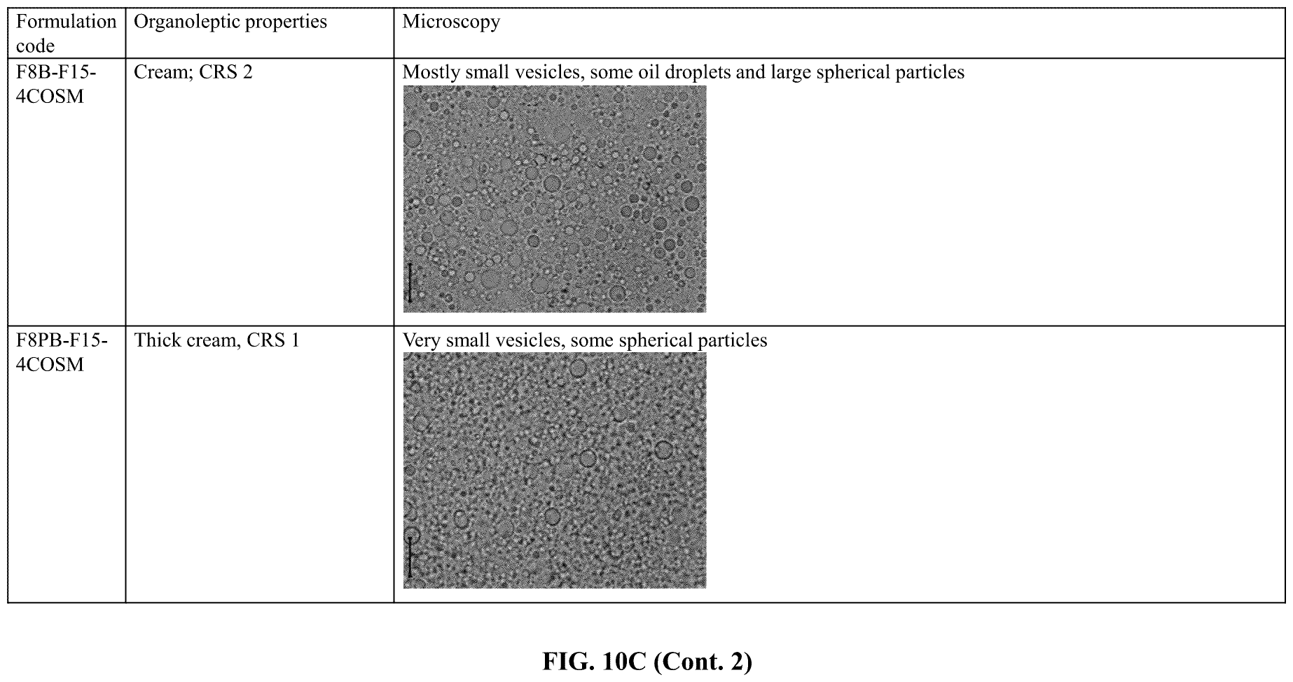

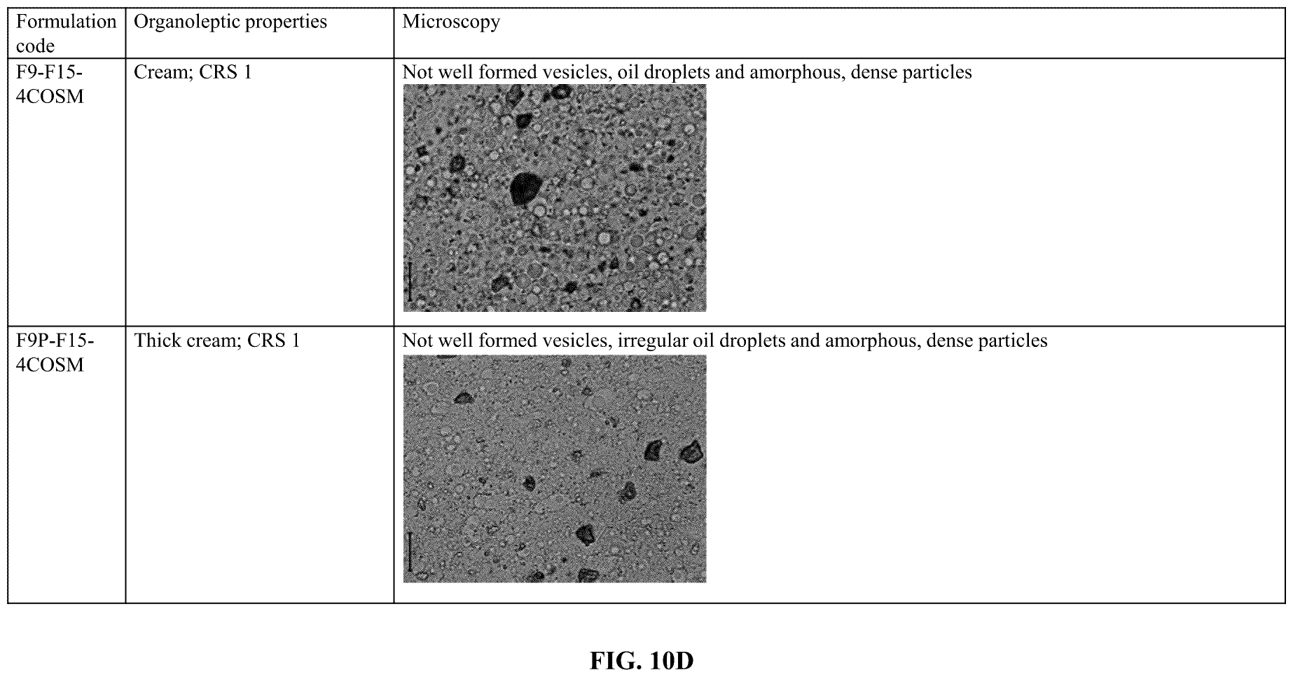

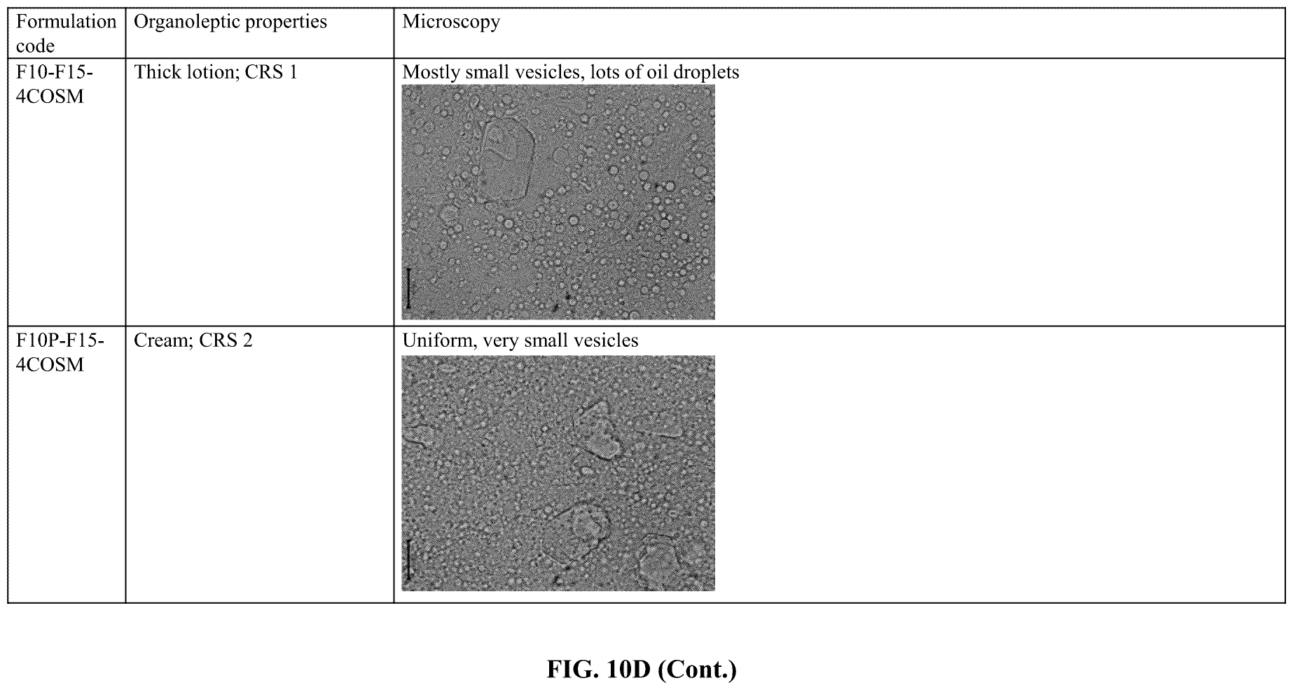

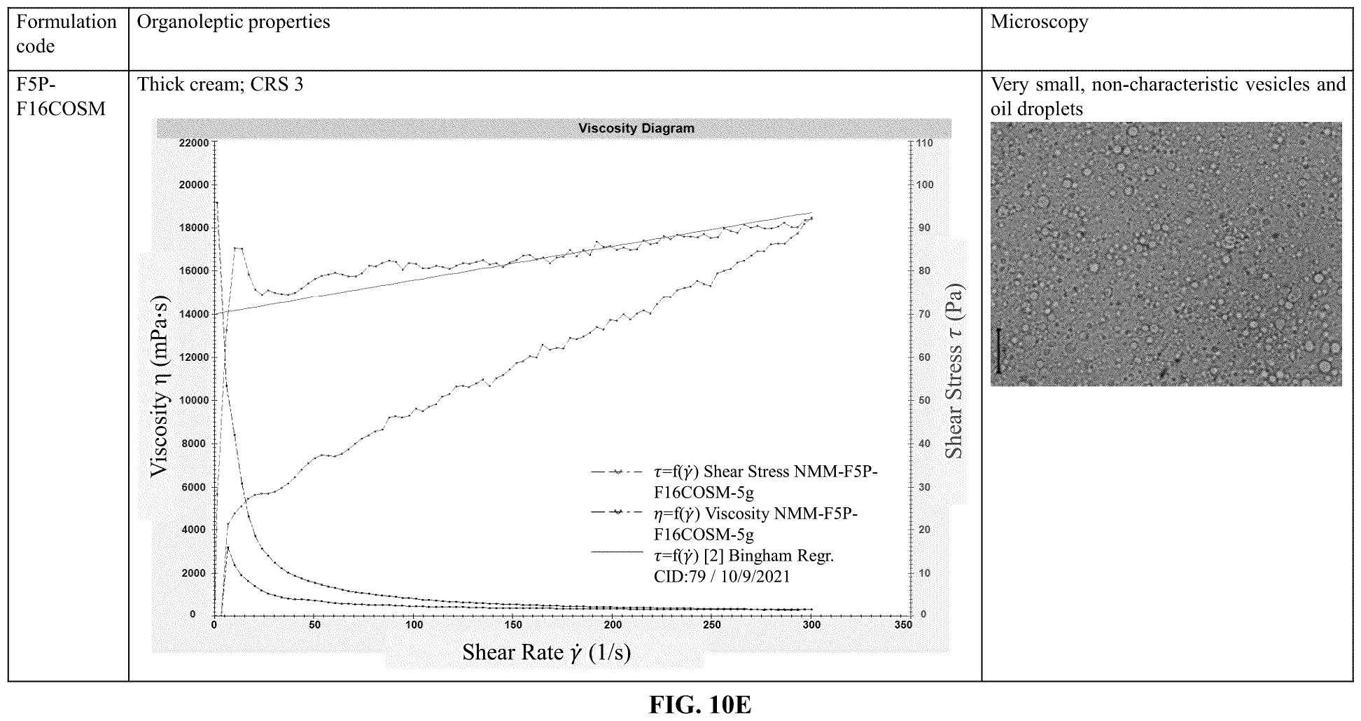

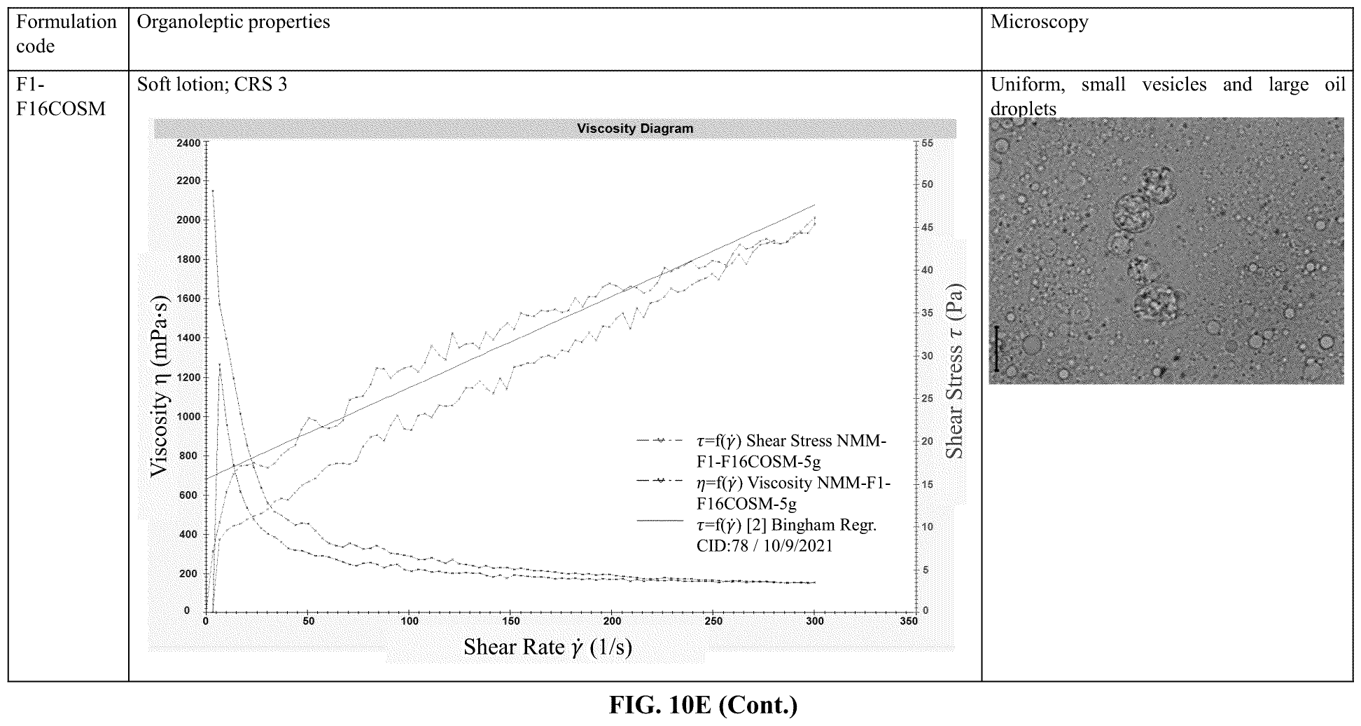

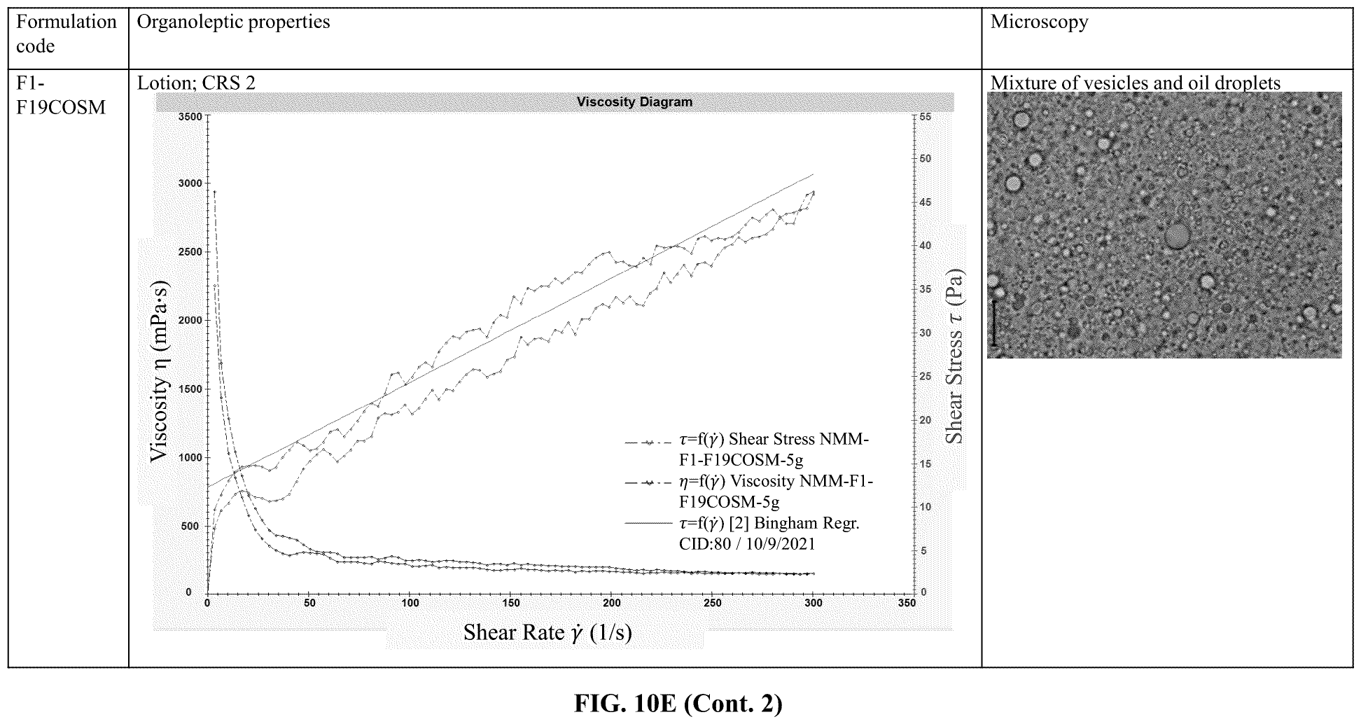

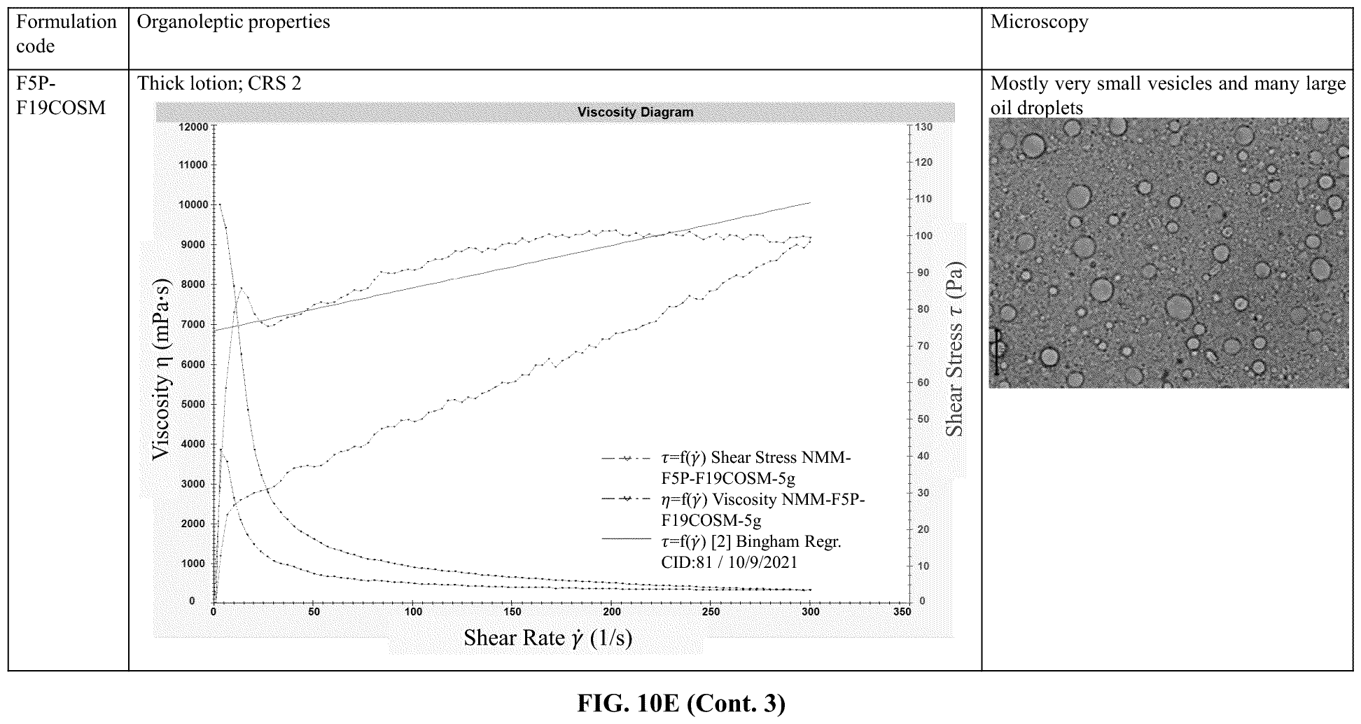

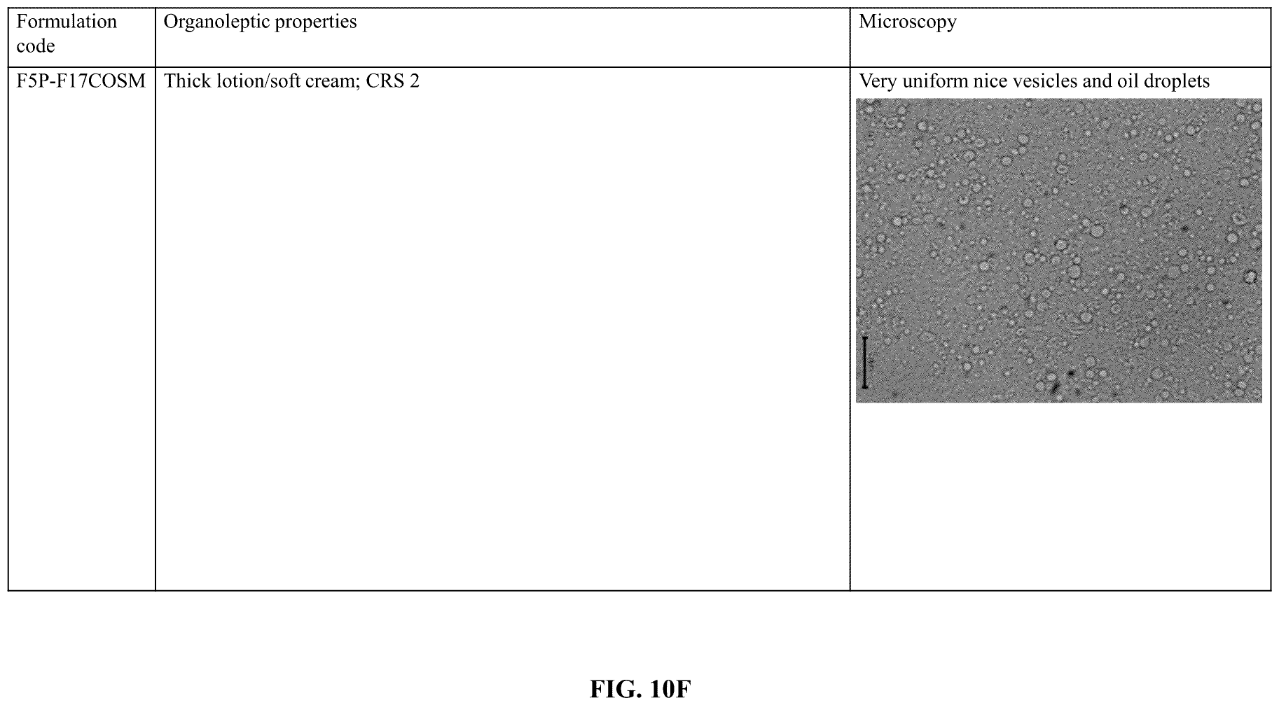

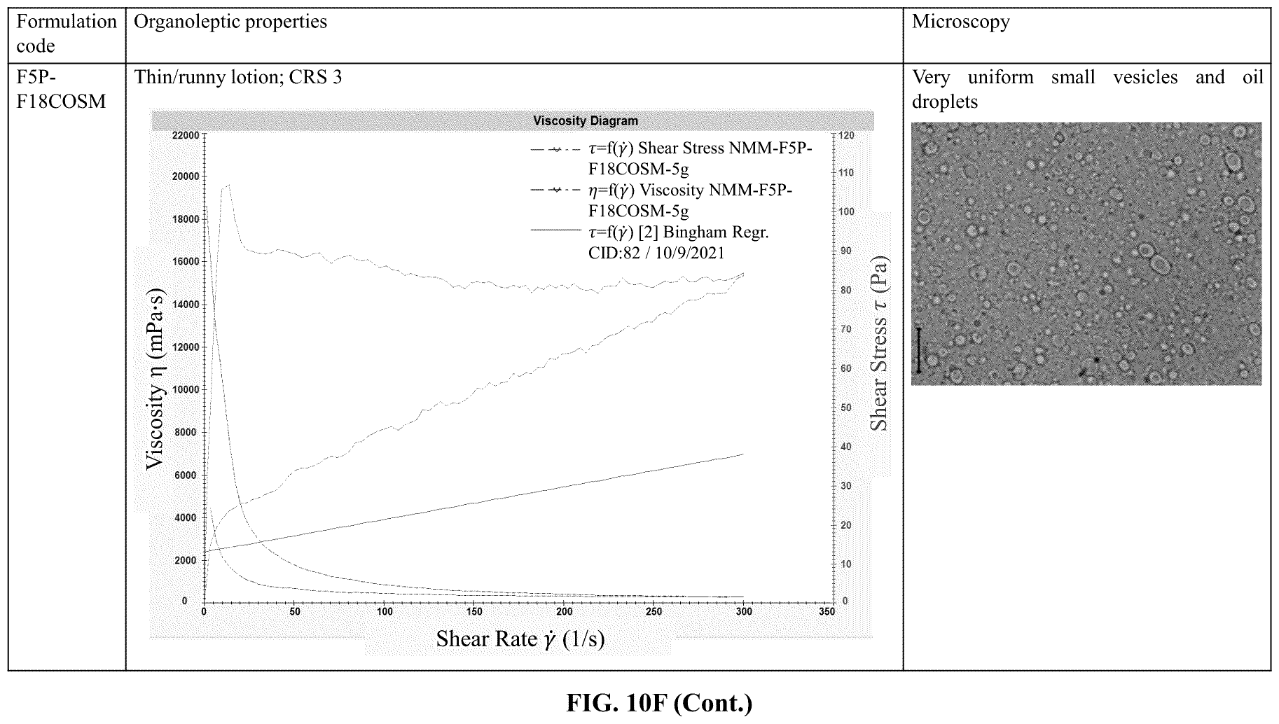

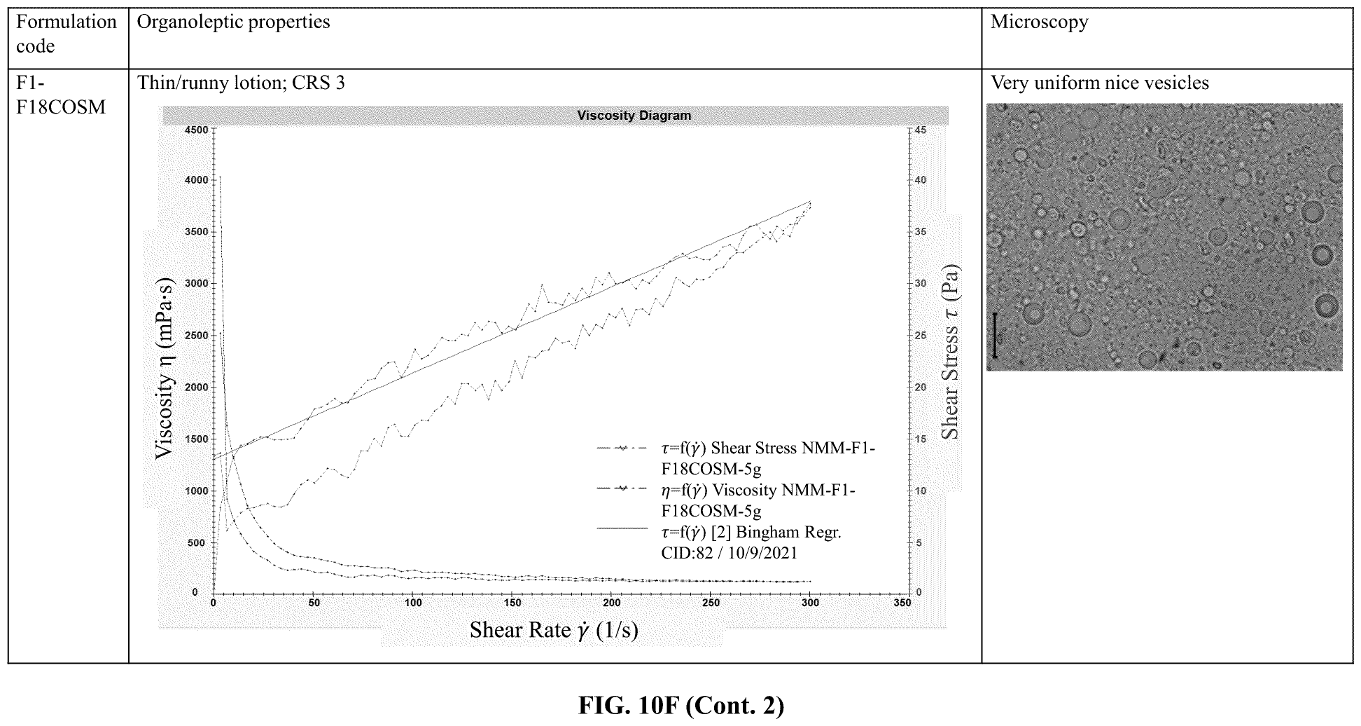

The various formulations were evaluated using viscosity (organoleptic properties) as well as through microscopy (FIGS. 10A-F). The various formulations were assigned a cosmetic rating scale (CRS): 1 (somewhat unappealing), 2 (acceptable), and 3 (excellent). Out of the formulations evaluated in FIGS. 10A-F, five sets of multiphasic vesicle formulations comprising C7 (2 mg/g) were selected for a diffusion cell study to evaluate the best enhancer combinations. The F1 series represented cholesterol containing formulations in the phospholipid phase and the F5P series represented the vegan substituted Phytosterol containing formulations. The five sets selected included: F5P-F15COSM and F1-15COSM, F5P-F15-4COSM and 51-5154COSM, F5P-F16COSM and F1-F16COSM, F5P-F19COSM and F1-F19COSM, as well as F5P-F18COSM and F1-F18COSM.

A first diffusion cell study included the analysis of the average amount of peptide in viable skin, as well as average % absorbed into viable skin. The results are shown in Table Q.

TABLE Q
Diffusion study 1 of multiphasic vesicle formulations

| | | | Average | |
|---|---|---|---|---|
| | | | amount of | Average % |
| | | | C7 ng/cm² | absorbed |
| | | | in viable | into viable |
| | | | skin (10x | skin (10x |
| | Lipid phase | System A | stripped) | stripped) |
| F1-F15COSM | F1SunL | F15COSM | 1106 | 0.35 |
| F5P-F15COSM | F5P | F15COSM | 855 | 0.27 |
| F1-F15-4COSM | F1SunL | F15-4COSM | 1132 | 0.36 |
| F5P-F15-4COSM | F5P | F15-4COSM | 1524 | 0.48 |
| F1-F16COSM | F1SunL | F16 | 908 | 0.29 |
| F5P-F16COSM | F5P | F16 | 1762 | 0.57 |
| F1-F19COSM | F1SunL | F19COSM | 1693 | 0.54 |
| F5P-F19COSM | F5P | F19COSM | 521 | 0.17 |
| F1-F18COSM | F1SunL | F18COSM | 1529 | 0.49 |
| F5P-F18COSM | F5P | F18COSM | — | — |

Based on the results shown in Table Q, three formulations were selected for further evaluation: F5P-G15-4COSM, F5P-F16COSM, F5P-F18COSM. These formulations were prioritized based on delivery efficiency and the vegan composition.

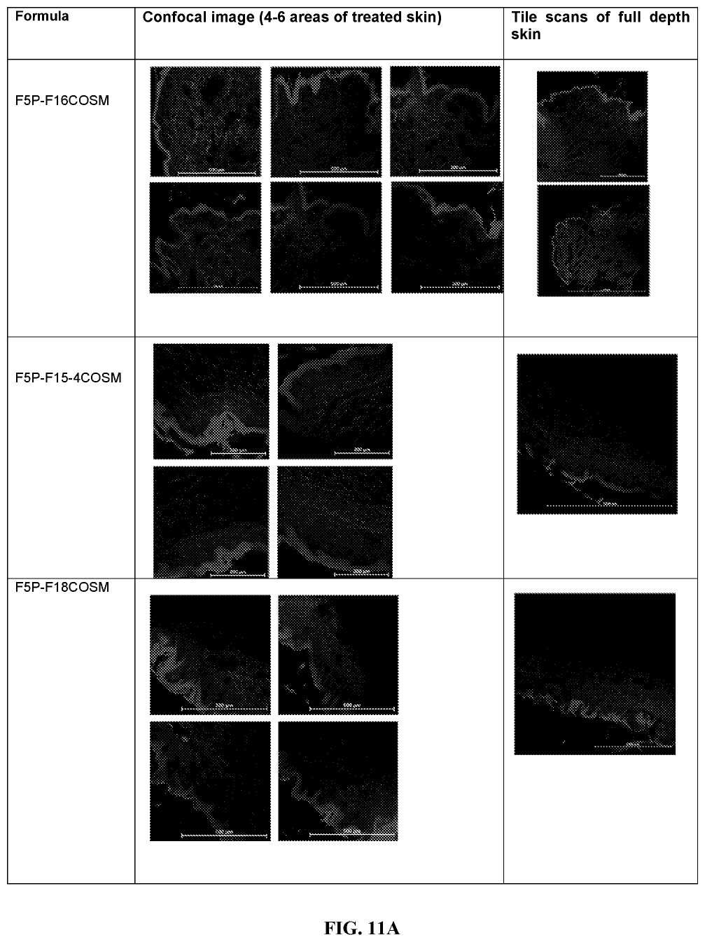

A second diffusion study was performed using confocal microscopy to evaluate peptide delivery in skin section. FIG. 11A shows the confocal microscopy images from the skin samples applied with F5P2.6-F16COSM, F5P2.6-F15-4COSM, and F5P2.6-F18COSM. These formulations contained Phytosterol MM. FIG. 11B shows the confocal microscopy images from the skin samples applied with F5P2.6-F16MCOSM, F5P2.6-F15-4MCOSM, and F5P2.6-F18MCOSM. The M in the formula codes indicated ‘modified’ composition with the addition of isopropyl myristate into System A to evaluate whether this addition provided an enhancer effect in the formulation.

Based on the results shown in FIGS. 11A-B, the F5P-F16COSM formula showed the highest levels of peptide delivery. Additionally, penetration of the peptides deep into the skin layers was observed. The peptide was also delivered across the full thickness of the skin. The F5P-F15-4COSM and F5P-F18COSM showed good delivery into the epidermal and upper dermal layers but not as deep. The addition (or increase) of isopropyl myristate into these formulations did not increase delivery, but rather seemed to decrease it in all three formulations. Here, F5P-F16COSM was selected as lead formulation.

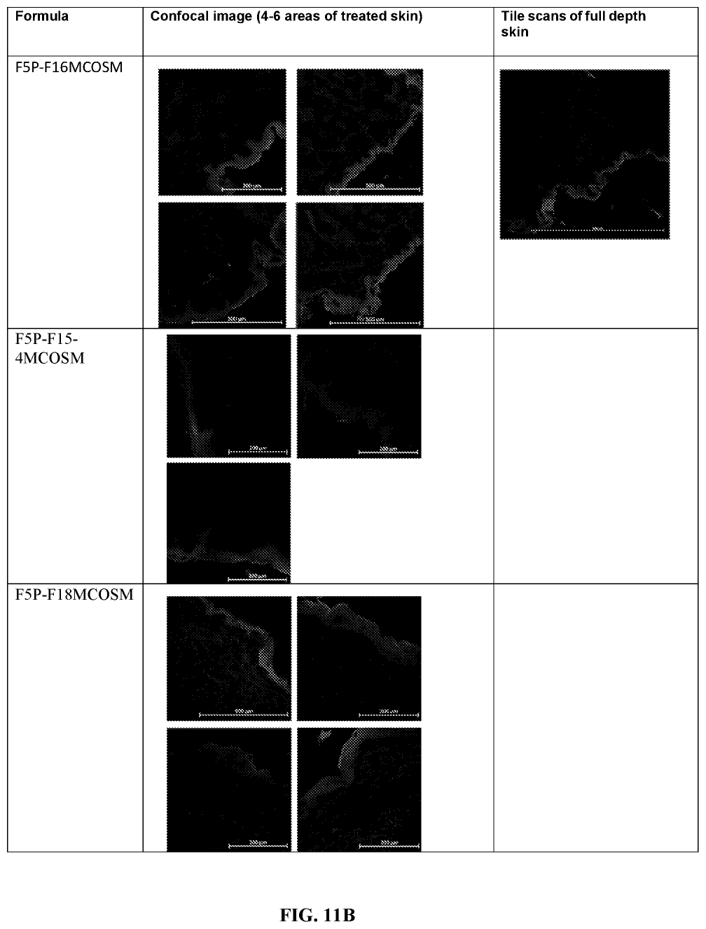

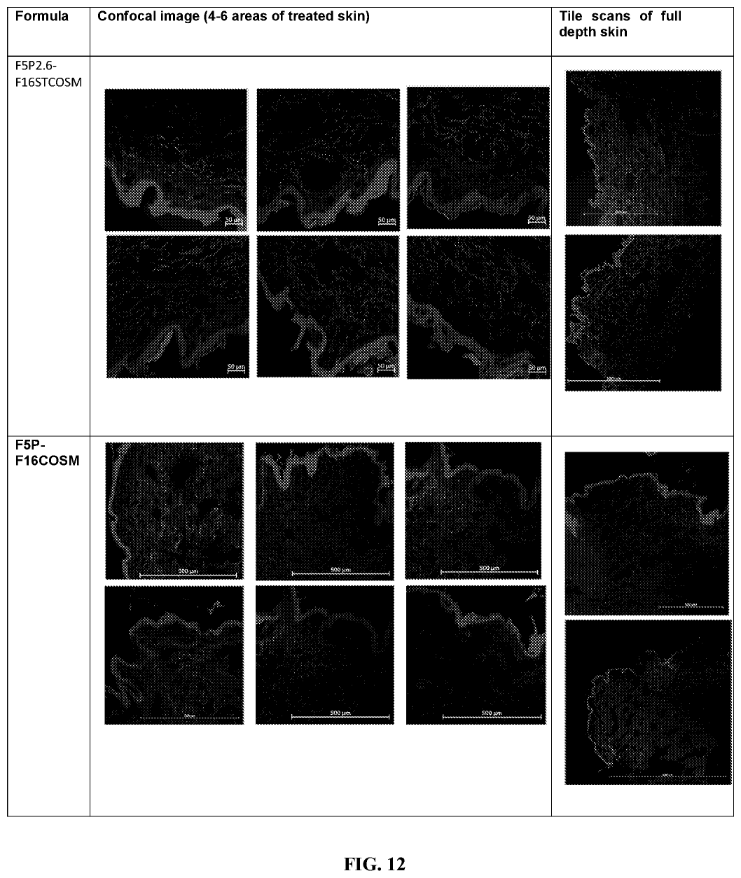

The lead formulation F5P2.6-F16COSM was further optimized: F5P2.6-F16STCOSM. The optimization was undertaken to increase the physical stability of the formulation. The optimization included the addition of one or more non-ionic surfactants described herein. These two formulations were compared to confirm that the optimization did not change the delivery efficiency. The two formulations were evaluated using confocal microscopy (FIG. 12), as well as through measuring the average epidermal and dermal fluorescence intensities of the two formulations (Table R). Both results indicated that the final formula was scalable, stable and effective in delivering C7 peptide.

TABLE R
fluorescence intensity values for epidermal
and dermal areas on cryosections

| | Average EPIDERMAL | Average DERMAL |
|---|---|---|
| | Fluorescence Intensity | Fluorescence Intensity |
| | n = 12 | n = 12 |
| F5P2.6- | 131 | 20 |
| F16COSM | (range 56-210) | (range 6-64) |
| F5P2.6- | 118 | 21 |
| F16STCOSM | (range 52-250) | (range 8-42) |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A lipid vesicle composition comprising:
(a) lipid vesicles each comprising a lipid bilayer comprising vesicle forming lipids,
(b) an oil-in-water emulsion entrapped in the lipid vesicles, and stabilized by one or more surfactants; and
(c) a peptide antagonist of muscle-type nicotinic acetylcholine receptors entrapped in the lipid bilayer and/or the oil-in-water emulsion.

2. The lipid vesicle composition of claim 1, wherein the peptide antagonist comprises an amino acid sequence at least about 50%, at least about 60%, at least about 70%, at least about 80%, or at least about 90% sequence homology to the amino acid sequence of any one of SEQ ID NOs: 1-52 or 60-99.

3. The lipid vesicle composition of claim 1, wherein the peptide antagonist comprises an amino acid sequence identical to the amino acid sequence of any one of SEQ ID NOs: 1-52 or 60-99.

4. The lipid vesicle composition of claim 1, wherein the peptide antagonist has an amino acid sequence consisting of an identical sequence to the amino acid sequence of any one of SEQ ID NOs: 1-52 or 60-99.

5. The lipid vesicle composition of claim 1, wherein the peptide antagonist has an amino acid sequence consisting of an identical sequence to the amino acid sequence of SEQ ID NO: 60, 61, 73, 78, 82, 85, 91, or 95.

6. The lipid vesicle composition of claim 1, wherein the peptide antagonist has an amino acid sequence consisting of an identical sequence to the amino acid sequence of SEQ ID NO: 60.

7. The lipid vesicle composition of claim 1, wherein the peptide antagonist has an amino acid sequence consisting of an identical sequence to the amino acid sequence of SEQ ID NO: 61.

8. The lipid vesicle composition of claim 1, wherein the peptide antagonist has an amino acid sequence consisting of an identical sequence to the amino acid sequence of SEQ ID NO: 73.

9. The lipid vesicle composition of claim 1, wherein the peptide antagonist has an amino acid sequence consisting of an identical sequence to the amino acid sequence of SEQ ID NO: 78.

10. The lipid vesicle composition of claim 1, wherein the peptide antagonist has an amino acid sequence consisting of an identical sequence to the amino acid sequence of SEQ ID NO: 82.

11. The lipid vesicle composition of claim 1, wherein the peptide antagonist has an amino acid sequence consisting of an identical sequence to the amino acid sequence of SEQ ID NO: 85.

12. The lipid vesicle composition of claim 1, wherein the peptide antagonist has an amino acid sequence consisting of an identical sequence to the amino acid sequence of SEQ ID NO: 91.

13. The lipid vesicle composition of claim 1, wherein the peptide antagonist has an amino acid sequence consisting of an identical sequence to the amino acid sequence of SEQ ID NO: 95.

14. The lipid vesicle composition of claim 1, wherein the peptide antagonist is present at a concentration of from about 0.1 mg/mL to about 10 mg/mL.

15. The lipid vesicle composition of claim 1, further comprising one or more penetration enhancing agents.

16. The lipid vesicle composition of claim 15, wherein the one or more penetration enhancing agents comprises a non-ionic surfactant or a combination of non-ionic surfactants.

17. The lipid vesicle composition of claim 16, wherein the non-ionic surfactant or combination of non-ionic surfactants is selected from polyethylene glycol ethers of fatty alcohols, sorbitan esters, polysorbates, sorbitan esters and polyethylene glycol fatty acid esters and combinations thereof.

18. The lipid vesicle composition of claim 17, wherein the polyethylene glycol ethers of fatty alcohols comprise a C₅-C₂₂ fatty alcohol and a polyethylene glycol group having from about 2 to about 8 ethylene glycol subunits.

19. The lipid vesicle composition of claim 17, wherein the polyethylene glycol ethers of fatty alcohols comprise diethylene glycol hexadecyl ether, 2-(2-octadecoxyethoxy)ethanol, diethylene glycol monooleyl ether, polyoxyethylene (3) oleyl ether, or polyoxyethylene (5) oleyl ether, or any combination thereof.

20. The lipid vesicle composition of claim 17, wherein the sorbitan esters comprise sorbitan monolaurate, sorbitan monopalmitate, sorbitan monostearate, sorbitan monooleate, sorbitan trioleate, sorbitan sesquioleate, or sorbitan isostearate, or any combinations thereof.

21. The lipid vesicle composition of claim 17, wherein the polyethylene glycol fatty acid ester comprises PEG-8 dilaurate, PEG-4 dilaurate, PEG-4 laurate, PEG-8 dioleate, PEG-8 distearate, PEG-8 distearate, PEG-7 glyceryl cocoate, and PEG-20 almond glycerides, or any combination thereof.

22. The lipid vesicle composition of claim 17, wherein the polysorbate comprises polysorbate 20, polysorbate 21, polysorbate 40, polysorbate 60, polysorbate 80, polysorbate 85, or any combination thereof.

23. The lipid vesicle composition of claim 17, wherein each of the non-ionic surfactants has a hydrophobic-lipophilic balance (HLB) of about 10 or less.

24. The lipid vesicle composition of claim 17, wherein the non-ionic surfactant or combination of non-ionic surfactants is present in an amount of from about 0.5% to about to about 10% (w/w) of the composition.


SEQUENCE LISTING (99 sequences)

SEQ ID NO: 1 - protein, 13 aa, synthetic construct
  REGION 1..13: note = Description of Artificial Sequence: Synthetic peptide
ECCNPACGRHYSC

SEQ ID NO: 2 - protein, 13 aa, synthetic construct
  REGION 1..13: note = Description of Artificial Sequence: Synthetic peptide
ECCNPACGKHFSC

SEQ ID NO: 3 - protein, 14 aa, synthetic construct
  REGION 1..14: note = Description of Artificial Sequence: Synthetic peptide
GRCCHPACGKNYSC

SEQ ID NO: 4 - protein, 12 aa, synthetic construct
  REGION 1..12: note = Description of Artificial Sequence: Synthetic peptide
CCKPACGKNYSC

SEQ ID NO: 5 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXRPACGHNYSX

SEQ ID NO: 6 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXHPACGHNYSX

SEQ ID NO: 7 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXKPAXGKQYSX

SEQ ID NO: 8 - protein, 12 aa, synthetic construct
  REGION 1..12: note = Description of Artificial Sequence: Synthetic peptide
CCRPACGKQYSC

SEQ ID NO: 9 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGKQYSC

SEQ ID NO: 10 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGRQYSX

SEQ ID NO: 11 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXHPACGRNYSX

SEQ ID NO: 12 - protein, 12 aa, synthetic construct
  REGION 1..12: note = Description of Artificial Sequence: Synthetic peptide
CCRPACGRNYSC

SEQ ID NO: 13 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXKPAXGRNYSX

SEQ ID NO: 14 - protein, 12 aa, synthetic construct
  REGION 1..12: note = Description of Artificial Sequence: Synthetic peptide
  MOD_RES 1: note = Selenocysteine
  MOD_RES 2: note = Cystathionine
  MOD_RES 6: note = Selenocysteine
  MOD_RES 12: note = Cystathionine
XXKPAXGKNYSX

SEQ ID NO: 15 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGKNYSX

SEQ ID NO: 16 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGKNYSX

SEQ ID NO: 17 - protein, 12 aa, synthetic construct
  REGION 1..12: note = Description of Artificial Sequence: Synthetic peptide
  MOD_RES 1: note = Selenocysteine
  MOD_RES 2: note = Selenocysteine
  MOD_RES 6: note = Selenocysteine
  MOD_RES 12: note = Selenocysteine
XXNPAXGRHYSX

SEQ ID NO: 18 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCNPAXGRHYSC

SEQ ID NO: 19 - protein, 12 aa, synthetic construct
  REGION 1..12: note = Description of Artificial Sequence: Synthetic peptide
CCQPACGKHYSC

SEQ ID NO: 20 - protein, 12 aa, synthetic construct
  REGION 1..12: note = Description of Artificial Sequence: Synthetic peptide
  MOD_RES 2: note = Cystathionine
  MOD_RES 6: note = Cystathionine
  MOD_RES 12: note = Cystathionine
CXNPAXGKHYSX

SEQ ID NO: 21 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXNPAXGRHYSX

SEQ ID NO: 22 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCNPAXGRHYSC

SEQ ID NO: 23 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXNPACGKHYSX

SEQ ID NO: 24 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXNPAXGKHYSX

SEQ ID NO: 25 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXNPAXGRHYSX

SEQ ID NO: 26 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXNPAXGRHYSX

SEQ ID NO: 27 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
RXXHPAXGKNYSX

SEQ ID NO: 28 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
RXXHPAXGKNYSX

SEQ ID NO: 29 - protein, 13 aa, synthetic construct
  REGION 1..13: note = Description of Artificial Sequence: Synthetic peptide
RCCHPACGKNYSC

SEQ ID NO: 30 - protein, 13 aa, synthetic construct
  REGION 1..13: note = Description of Artificial Sequence: Synthetic peptide
  MOD_RES 3: note = Cystathionine
  MOD_RES 13: note = Cystathionine
RCXKPACGKNYSX

SEQ ID NO: 31 - protein, 13 aa, synthetic construct
  REGION 1..13: note = Description of Artificial Sequence: Synthetic peptide
  MOD_RES 3: note = Cystathionine
  MOD_RES 13: note = Cystathionine
RCXHPACGKNYSX

SEQ ID NO: 32 - protein, 13 aa, synthetic construct
  REGION 1..13: note = Description of Artificial Sequence: Synthetic peptide
  MOD_RES 2: note = Cystathionine
  MOD_RES 7: note = Cystathionine
RXCHPAXGRNYSC

SEQ ID NO: 33 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
RXXRPAXGKNYSX

SEQ ID NO: 34 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
RXXHPAXGHNYSX

SEQ ID NO: 35 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
RXXRPAXGKNYSX

SEQ ID NO: 36 - protein, 13 aa, synthetic construct
  REGION 1..13: note = Description of Artificial Sequence: Synthetic peptide
  MOD_RES 2: note = Selenocysteine
  MOD_RES 3: note = Selenocysteine
  MOD_RES 7: note = Selenocysteine
  MOD_RES 13: note = Selenocysteine
RXXHPAXGKNYSX

SEQ ID NO: 37 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
KXXHPAXGKNYSX

SEQ ID NO: 38 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
KXXHPAXGRNYSX

SEQ ID NO: 39 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGRHYSX

SEQ ID NO: 40 - protein, 12 aa, synthetic construct
  REGION 1..12: note = Description of Artificial Sequence: Synthetic peptide
CCKPACGRHYSC

SEQ ID NO: 41 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXHPACGRHYSX

SEQ ID NO: 42 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXHPACGRHYSX

SEQ ID NO: 43 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGKHYSX

SEQ ID NO: 44 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGRHYSX

SEQ ID NO: 45 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGKHYSX

SEQ ID NO: 46 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGRKYSC

SEQ ID NO: 47 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGRKYSC

SEQ ID NO: 48 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGRKYSC

SEQ ID NO: 49 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGRHYSX

SEQ ID NO: 50 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXKPAXGRHYSX

SEQ ID NO: 51 - protein, 12 aa, synthetic construct
  REGION 1..12: note = Description of Artificial Sequence: Synthetic peptide
  MOD_RES 1: note = Selenocysteine
  MOD_RES 2: note = Cystathionine
  MOD_RES 6: note = Selenocysteine
  MOD_RES 12: note = Cystathionine
XXHPAXGKHYSX

SEQ ID NO: 52 - protein, 12 aa, synthetic construct
  REGION 1..12: note = Description of Artificial Sequence: Synthetic peptide
  MOD_RES 1: note = Cystathionine
  MOD_RES 2: note = Selenocysteine
  MOD_RES 6: note = Cystathionine
  MOD_RES 12: note = Selenocysteine
XXHPAXGRHYSX

SEQ ID NO: 53-55 - skipped (3 entries)

SEQ ID NO: 56 - protein, 13 aa, synthetic construct
  REGION 1..13: note = Description of Artificial Sequence: Synthetic peptide
ECCHPACGKHFSC

SEQ ID NO: 57-59 - skipped (3 entries)

SEQ ID NO: 60 - protein, 12 aa, synthetic construct
  REGION 1..12: note = Description of Artificial Sequence: Synthetic peptide
CCNPACGKNYSC

SEQ ID NO: 61 - protein, 12 aa, synthetic construct
  REGION 1..12: note = Description of Artificial Sequence: Synthetic peptide
CCHPACGRHYSC

SEQ ID NO: 62 - protein, 12 aa, synthetic construct
  REGION 1..12: note = Description of Artificial Sequence: Synthetic peptide
CCNPACGKNYKC

SEQ ID NO: 63 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXNPAXGKNYSX

SEQ ID NO: 64 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGRHYSX

SEQ ID NO: 65 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXNPAXGKNYKX

SEQ ID NO: 66 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXNPACGKNYSX

SEQ ID NO: 67 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXHPACGRHYSX

SEQ ID NO: 68 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXNPACGKNYKX

SEQ ID NO: 69 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCNPAXGKNYSC

SEQ ID NO: 70 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGRHYSC

SEQ ID NO: 71 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCNPAXGKNYKC

SEQ ID NO: 72 - protein, 13 aa, synthetic construct
  REGION 1..13: note = Description of Artificial Sequence: Synthetic peptide
  MOD_RES 2: note = Cystathionine
  MOD_RES 7: note = Cystathionine
RXCHPAXGKNYSC

SEQ ID NO: 73 - protein, 13 aa, synthetic construct
  REGION 1..13: note = Description of Artificial Sequence: Synthetic peptide
  MOD_RES 3: note = Cystathionine
  MOD_RES 13: note = Cystathionine
RCXHPACGKNYSX

SEQ ID NO: 74 - protein, 13 aa, synthetic construct
  REGION 1..13: note = Description of Artificial Sequence: Synthetic peptide
RCCHPACGKNYSC

SEQ ID NO: 75 - protein, 13 aa, synthetic construct
  REGION 1..13: note = Description of Artificial Sequence: Synthetic peptide
  MOD_RES 2: note = Cystathionine
  MOD_RES 3: note = Cystathionine
  MOD_RES 7: note = Cystathionine
  MOD_RES 13: note = Cystathionine
RXXHPAXGKNYSX

SEQ ID NO: 76 - protein, 12 aa, synthetic construct
  REGION 1..12: note = Description of Artificial Sequence: Synthetic peptide
  MOD_RES 2: note = Cystathionine
  MOD_RES 12: note = Cystathionine
CXHPACGKNYSX

SEQ ID NO: 77 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXHPAXGKNYSX

SEQ ID NO: 78 - protein, 12 aa, synthetic construct
  REGION 1..12: note = Description of Artificial Sequence: Synthetic peptide
CCHPACGKNYSC

SEQ ID NO: 79 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGKNYSC

SEQ ID NO: 80 - protein, 12 aa, synthetic construct
  REGION 1..12: note = Description of Artificial Sequence: Synthetic peptide
CCKPACGRHYSC

SEQ ID NO: 81 - protein, 12 aa, synthetic construct
  REGION 1..12: note = Description of Artificial Sequence: Synthetic peptide
CCHPACGRHYSC

SEQ ID NO: 82 - protein, 12 aa, synthetic construct
  REGION 1..12: note = Description of Artificial Sequence: Synthetic peptide
CCHPACGRNYSC

SEQ ID NO: 83 - protein, 12 aa, synthetic construct
  REGION 1..12: note = Description of Artificial Sequence: Synthetic peptide
CCKPACGRNYSC

SEQ ID NO: 84 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGRNYSC

SEQ ID NO: 85 - protein, 12 aa, synthetic construct
  REGION 1..12: note = Description of Artificial Sequence: Synthetic peptide
CCHPACGKHYSC

SEQ ID NO: 86 - protein, 12 aa, synthetic construct
  REGION 1..12: note = Description of Artificial Sequence: Synthetic peptide
CCHPACGRKYSC

SEQ ID NO: 87 - protein, 12 aa, synthetic construct
  REGION 1..12: note = Description of Artificial Sequence: Synthetic peptide
  MOD_RES 1: note = Cystathionine
  MOD_RES 2: note = Cystathionine
  MOD_RES 6: note = Cystathionine
  MOD_RES 12: note = Cystathionine
XXKPAXGRHYSX

SEQ ID NO: 88 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCHPAXGKHYSC

SEQ ID NO: 89 - protein, 12 aa, synthetic construct
  REGION 1..12: note = Description of Artificial Sequence: Synthetic peptide
CCHPACGKHYSC

SEQ ID NO: 90 - protein, 12 aa, synthetic construct
  REGION 1..12: note = Description of Artificial Sequence: Synthetic peptide
CCNPACGRHYSC

SEQ ID NO: 91 - protein, 12 aa, synthetic construct
  REGION 1..12: note = Description of Artificial Sequence: Synthetic peptide
CCNPACGRNYSC

SEQ ID NO: 92 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCNPAXGRHYSC

SEQ ID NO: 93 - protein, 12 aa, synthetic construct
  REGION 1..12: note = Description of Artificial Sequence: Synthetic peptide
CCNPACGRNYSC

SEQ ID NO: 94 - protein, 12 aa, synthetic construct
  REGION 1..12: note = Description of Artificial Sequence: Synthetic peptide
CCKPGCGRHYSC

SEQ ID NO: 95 - protein, 12 aa, synthetic construct
  REGION 1..12: note = Description of Artificial Sequence: Synthetic peptide
CCNPACGKHYSC

SEQ ID NO: 96 - protein, 12 aa, synthetic construct
  REGION 1..12: note = Description of Artificial Sequence: Synthetic peptide
CCKPACGKHYSC

SEQ ID NO: 97 - protein, 12 aa, synthetic construct
  REGION 1..12: note = Description of Artificial Sequence: Synthetic peptide
CCNPACGKHYSC

SEQ ID NO: 98 - protein, 12 aa, synthetic construct
  REGION 1..12: note = Description of Artificial Sequence: Synthetic peptide
CCNPACGHKYSC

SEQ ID NO: 99 - protein, 12 aa, synthetic construct
  REGION 1..12: note = Description of Artificial Sequence: Synthetic peptide
  MOD_RES 1: note = Cystathionine
  MOD_RES 6: note = Cystathionine
XCNPAXGKHYSC